SEQUENCE LISTING (148 sequences)

SEQ ID NO: 1 - RNA, 31 nt, Homo sapiens
ggccagaucugagccugggagcucucuggcc

SEQ ID NO: 2 - RNA, 26 nt, Homo sapiens
ccaaaggcucuucucagagccaccca

SEQ ID NO: 3 - protein, 369 aa, Artificial Sequence
  Description of Artificial Sequence Synthetic polypeptide
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNS

SEQ ID NO: 4 - protein, 40 aa, Artificial Sequence
  Description of Artificial Sequence Synthetic polypeptide
ITSLYKKAGSETVRFQSHHHHHHSSGVDLGTENLYFQSNA

SEQ ID NO: 5 - protein, 90 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEE

SEQ ID NO: 6 - protein, 18 aa, Artificial Sequence
  Description of Artificial Sequence Synthetic peptide
GGSGGSGGSGGSGGSGGS

SEQ ID NO: 7 - protein, 102 aa, Artificial Sequence
  Description of Artificial Sequence Synthetic polypeptide
MAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFV

SEQ ID NO: 8 - protein, 11 aa, Artificial Sequence
  Description of Artificial Sequence Synthetic peptide
LQLPPLERLTL

SEQ ID NO: 9 - protein, 181 aa, Artificial Sequence
  Description of Artificial Sequence Synthetic polypeptide
MELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVG

SEQ ID NO: 10 - protein, 6 aa, Artificial Sequence
  Description of Artificial Sequence Synthetic 6xHis tag
HHHHHH

SEQ ID NO: 11 - protein, 181 aa, Artificial Sequence
  Description of Artificial Sequence Synthetic polypeptide
MELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNADLILSCCLHYCKDKAKDFMPASKEEPIRLLREVVLLTDDRNLRVKALTRNVPVRDIPAFLTWAQVG

SEQ ID NO: 12 - protein, 22 aa, Artificial Sequence
  Description of Artificial Sequence Synthetic peptide
DYKDHDGDYKDHDIDYKDDDDK

SEQ ID NO: 13 - protein, 16 aa, Artificial Sequence
  Description of Artificial Sequence Synthetic peptide
KRPAATKKAGQAKKKK

SEQ ID NO: 14 - protein, 8 aa, Artificial Sequence
  Description of Artificial Sequence Synthetic peptide
DYKDDDDK

SEQ ID NO: 15 - protein, 364 aa, Artificial Sequence
  Description of Artificial Sequence Synthetic polypeptide
MSATSVDTQRTKGQDNKVQNGSLHQKDTVHDNDFEPYLTGQSNQSNSYPSMSDPYLSSYYPPSIGFPYSLNEAPWSTAGDPPIPYLTTYGQLSNGDHHFMHDAVFGQPGGLGNNIYQHRFNFFPENPAFSAWGTSGSQGQQTQSSAYGSSYTYPPSSLGGTVVDGQPGFHSDTLSKAPGMNSLEQGMVGLKIGDVSSSAVKTVGSVVSSVALTGVLSGNGGTNVNMPVSKPTSWAAIASKPAKPQPKMKTKSGPVMGGGLPPPPIKHNMDIGTWDNKGPVPKAPVPQQAPSPQAAPQPQQVAQPLPAQPPALAQPQYQSPQQPPQTRWVAPRNRNAAFGQSGGAGSDSNSPGNVQPNSAPSVES

SEQ ID NO: 16 - protein, 90 aa, Artificial Sequence
  Description of Artificial Sequence Synthetic polypeptide
DAMFGQPGALGSTPFLGQHGFNFFPSGIDFSAWGNNSSQGQSTQSSGYSSNYAYAPSSLGGAMIDGQSAFANETLNKAPGMNTIDQGMAA

SEQ ID NO: 17 - protein, 191 aa, Artificial Sequence
  Description of Artificial Sequence Synthetic polypeptide
MSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAA

SEQ ID NO: 18 - protein, 73 aa, Artificial Sequence
  Description of Artificial Sequence Synthetic polypeptide
ADFETDESVLMRRQKQINYGKNTIAYDRYIKEVPRHLRQPGIHPKTPNKFKKYSRRSWDQQIKLWKVALHFWD

SEQ ID NO: 19 - protein, 76 aa, Artificial Sequence
  Description of Artificial Sequence Synthetic polypeptide
MRVTLSSKPQALATPNKEEHGKRKKKGKGLGKKRDPCLRKYKDFCIHGECKYVKELRAPSCICHPGYHGERCHGLS

SEQ ID NO: 20 - protein, 46 aa, Artificial Sequence
  Description of Artificial Sequence Synthetic polypeptide
MGIINTLQKYYCRVRGGRCAVLSCLPKEEQIGKCSTRGRKCCRRKK

SEQ ID NO: 21 - protein, 9 aa, Artificial Sequence
  Description of Artificial Sequence Synthetic peptide
GGSGGSGGS

SEQ ID NO: 22 - protein, 16 aa, Artificial Sequence
  Description of Artificial Sequence Synthetic peptide
SGSETPGTSESATPES

SEQ ID NO: 23 - protein, 4 aa, Artificial Sequence
  Description of Artificial Sequence Synthetic peptide
GGSG

SEQ ID NO: 24 - protein, 73 aa, Artificial Sequence
  Description of Artificial Sequence Synthetic polypeptide
EEEEKKKQQEEEAERLRRIQEEMEKERKRREEDEQRRRKEEEERRMKLEMEAKRKQEEEERKKREDDEKRKKK

SEQ ID NO: 25 - protein, 5 aa, Artificial Sequence
  Description of Artificial Sequence Synthetic peptide
GSGGS

SEQ ID NO: 26 - DNA, 31 bp, Artificial Sequence
  Description of Artificial Sequence Synthetic oligonucleotide
ggccagatctgagcctgggagctctctggcc

SEQ ID NO: 27 - DNA, 40 bp, Artificial Sequence
  Description of Artificial Sequence Synthetic oligonucleotide
tgacagcccacatggcattccacttatcactggcatcctt

SEQ ID NO: 28 - DNA, 26 bp, Artificial Sequence
  Description of Artificial Sequence Synthetic oligonucleotide
ccaaaggctcttctcagagccaccca

SEQ ID NO: 29 - DNA, 20 bp, Artificial Sequence
  Description of Artificial Sequence Synthetic oligonucleotide
gtgataagtggaatgccatg

SEQ ID NO: 30 - DNA, 40 bp, Artificial Sequence
  Description of Artificial Sequence Synthetic oligonucleotide
caggtcgactctagactcgaggctagcgagctcgtttaaa

SEQ ID NO: 31 - DNA, 40 bp, Artificial Sequence
  Description of Artificial Sequence Synthetic oligonucleotide
cttggtgctctccaccttccgcaccacctccatgccctct

SEQ ID NO: 32 - DNA, 40 bp, Artificial Sequence
  Description of Artificial Sequence Synthetic oligonucleotide
ccgatgcggtgggctcggtcctgcgcttgcaggtcctggt

SEQ ID NO: 33 - DNA, 40 bp, Artificial Sequence
  Description of Artificial Sequence Synthetic oligonucleotide
gcctccaccagctctctgactgtacccccagagacctcat

SEQ ID NO: 34 - DNA, 40 bp, Artificial Sequence
  Description of Artificial Sequence Synthetic oligonucleotide
aagcatcttcaacaccctgtctggtcttggctgaggtttc

SEQ ID NO: 35 - DNA, 40 bp, Artificial Sequence
  Description of Artificial Sequence Synthetic oligonucleotide
cttcgcaccgcagcgcagcgcggctcagctcccggctcgt

SEQ ID NO: 36 - DNA, 20 bp, Artificial Sequence
  Description of Artificial Sequence Synthetic oligonucleotide
ctagactcgaggctagcgag

SEQ ID NO: 37 - DNA, 30 bp, Artificial Sequence
  Description of Artificial Sequence Synthetic oligonucleotide
cgactctagactcgaggctagcgagctcgt

SEQ ID NO: 38 - DNA, 40 bp, Artificial Sequence
  Description of Artificial Sequence Synthetic oligonucleotide
ggccctggcccttcccctggagccatgctgggccctagcc

SEQ ID NO: 39 - DNA, 40 bp, Artificial Sequence
  Description of Artificial Sequence Synthetic oligonucleotide
gcccaaccccatttaaccagaaccagctgcaccagctcag

SEQ ID NO: 40 - DNA, 40 bp, Artificial Sequence
  Description of Artificial Sequence Synthetic oligonucleotide
cctcccaagccctggcctgaaggacccatggcgaatgctg

SEQ ID NO: 41 - DNA, 40 bp, Artificial Sequence
  Description of Artificial Sequence Synthetic oligonucleotide
cgtcccacccgccgcctcgcccgtgatgccaccgcagacc

SEQ ID NO: 42 - DNA, 40 bp, Artificial Sequence
  Description of Artificial Sequence Synthetic oligonucleotide
ggaggtggtggtgtgcatgcggagggacacagcgctggag

SEQ ID NO: 43 - DNA, 40 bp, Artificial Sequence
  Description of Artificial Sequence Synthetic oligonucleotide
tcacaggcaaaatccagaagctgaccaaggcagtggccac

SEQ ID NO: 44 - DNA, 40 bp, Artificial Sequence
  Description of Artificial Sequence Synthetic oligonucleotide
catggctgaagatgaggaggggtaccgcaagctcatcgac

SEQ ID NO: 45 - DNA, 40 bp, Artificial Sequence
  Description of Artificial Sequence Synthetic oligonucleotide
ctctggacgagaccagccagatgagcgacctcccggtgaa

SEQ ID NO: 46 - DNA, 40 bp, Artificial Sequence
  Description of Artificial Sequence Synthetic oligonucleotide
cccaccctgcccgtggaggagaagaagaagattccagatc

SEQ ID NO: 47 - DNA, 40 bp, Artificial Sequence
  Description of Artificial Sequence Synthetic oligonucleotide
aatatggcgtgtcccaggcccttgcacgtggcctgcagtc

SEQ ID NO: 48 - DNA, 40 bp, Artificial Sequence
  Description of Artificial Sequence Synthetic oligonucleotide
ctcatcacgtacctcatggagcacaaacgcatcaatgggc

SEQ ID NO: 49 - DNA, 40 bp, Artificial Sequence
  Description of Artificial Sequence Synthetic oligonucleotide
tggtgaaggtgtcttacaagggatccccagcagcaagacg

SEQ ID NO: 50 - DNA, 40 bp, Artificial Sequence
  Description of Artificial Sequence Synthetic oligonucleotide
accccgccgcctgctgctgacgggcacaccgctgcagaac

SEQ ID NO: 51 - DNA, 40 bp, Artificial Sequence
  Description of Artificial Sequence Synthetic oligonucleotide
cagtggtttaacgcaccctttgccatgaccggggaaaagg

SEQ ID NO: 52 - DNA, 40 bp, Artificial Sequence
  Description of Artificial Sequence Synthetic oligonucleotide
acgactcaagaaggaagtcgaggcccagttgcccgaaaag

SEQ ID NO: 53 - DNA, 40 bp, Artificial Sequence
  Description of Artificial Sequence Synthetic oligonucleotide
gtgctgctgactgatggctccgagaaggacaagaagggca

SEQ ID NO: 54 - DNA, 40 bp, Artificial Sequence
  Description of Artificial Sequence Synthetic oligonucleotide
ggaaccacgaaggcggaggaccggggcatgctgctgaaaa

SEQ ID NO: 55 - DNA, 40 bp, Artificial Sequence
  Description of Artificial Sequence Synthetic oligonucleotide
tccagtcggcagacactgtgatcatttttgacagcgactg

SEQ ID NO: 56 - DNA, 40 bp, Artificial Sequence
  Description of Artificial Sequence Synthetic oligonucleotide
cgccagcgggcgtcaaccccgacttggaggagccacctct

SEQ ID NO: 57 - DNA, 40 bp, Artificial Sequence
  Description of Artificial Sequence Synthetic oligonucleotide
gcggaggtggagcggctgacctgtgaggaggaggaggaga

SEQ ID NO: 58 - DNA, 40 bp, Artificial Sequence
  Description of Artificial Sequence Synthetic oligonucleotide
tgatcaagtacaaggacagcagcagtggacgtcagctcag

SEQ ID NO: 59 - DNA, 40 bp, Artificial Sequence
  Description of Artificial Sequence Synthetic oligonucleotide
cttcaagaagataaaggagcgcattcgcaaccacaagtac

SEQ ID NO: 60 - DNA, 40 bp, Artificial Sequence
  Description of Artificial Sequence Synthetic oligonucleotide
agggtcccgagccaagccggtcgtgagtgacgatgacagt

SEQ ID NO: 61 - DNA, 40 bp, Artificial Sequence
  Description of Artificial Sequence Synthetic oligonucleotide
tatttatacagcagagaagctgtaggactgtttgtgactg

SEQ ID NO: 62 - DNA, 40 bp, Artificial Sequence
  Description of Artificial Sequence Synthetic oligonucleotide
ggggaacacacgatacctgtttttcttttccgttgctggc

SEQ ID NO: 63 - DNA, 70 bp, Artificial Sequence
  Description of Artificial Sequence Synthetic oligonucleotide
ataggccagtgaattcgagctcgaatatggattacttggtagaacagcaatctacgccggaagcataaag

SEQ ID NO: 64 - DNA, 173 bp, Artificial Sequence
  Description of Artificial Sequence Synthetic polynucleotide
ggccagtgaattcgagctcggtacccggggatcctctagaaatatggattacttggtagaacagcaatctactcgacctgcaggcatgcaagcttggcgtaatcatggtcatagctgtttcctgtgtttatccgctcacaattccacacaacatacgagccggaagcataaag

SEQ ID NO: 65 - DNA, 60 bp, Artificial Sequence
  Description of Artificial Sequence Synthetic oligonucleotide
ggccagatctgagcctgggagctctctggcccagccaccacccacagagccgccaccaga

SEQ ID NO: 66 - DNA, 60 bp, Artificial Sequence
  Description of Artificial Sequence Synthetic oligonucleotide
ggccagatctgagcctgggagctctctggccctagattgctgttctaccaagtaatccat

SEQ ID NO: 67 - DNA, 22 bp, Artificial Sequence
  Description of Artificial Sequence Synthetic primer
gtctcctctgacttcaacagcg

SEQ ID NO: 68 - DNA, 22 bp, Artificial Sequence
  Description of Artificial Sequence Synthetic primer
accaccctgttgctgtagccaa

SEQ ID NO: 69 - DNA, 22 bp, Artificial Sequence
  Description of Artificial Sequence Synthetic primer
aacgcaggcaaagacaccaacg

SEQ ID NO: 70 - DNA, 22 bp, Artificial Sequence
  Description of Artificial Sequence Synthetic primer
tctgtcttggtgctctccacct

SEQ ID NO: 71 - DNA, 21 bp, Artificial Sequence
  Description of Artificial Sequence Synthetic primer
aggttacaaccgccaagaagc

SEQ ID NO: 72 - DNA, 21 bp, Artificial Sequence
  Description of Artificial Sequence Synthetic primer
atgagaatctcgcggatcttg

SEQ ID NO: 73 - DNA, 22 bp, Artificial Sequence
  Description of Artificial Sequence Synthetic primer
aacgcaggcaaagacaccaacg

SEQ ID NO: 74 - DNA, 22 bp, Artificial Sequence
  Description of Artificial Sequence Synthetic primer
tctgtcttggtgctctccacct

SEQ ID NO: 75 - DNA, 22 bp, Artificial Sequence
  Description of Artificial Sequence Synthetic primer
gcagcactacttcttgaccacc

SEQ ID NO: 76 - DNA, 22 bp, Artificial Sequence
  Description of Artificial Sequence Synthetic primer
tctgctcctgagcattgacgtc

SEQ ID NO: 77 - DNA, 22 bp, Artificial Sequence
  Description of Artificial Sequence Synthetic primer
gaaacctcagccaagaccagac

SEQ ID NO: 78 - DNA, 22 bp, Artificial Sequence
  Description of Artificial Sequence Synthetic primer
ggcaatcccatacaaccctgag

SEQ ID NO: 79 - DNA, 22 bp, Artificial Sequence
  Description of Artificial Sequence Synthetic primer
tgaggctgctttcactatccgc

SEQ ID NO: 80 - DNA, 22 bp, Artificial Sequence
  Description of Artificial Sequence Synthetic primer
gaggaaggtcttggtggcaatc

SEQ ID NO: 81 - DNA, 22 bp, Artificial Sequence
  Description of Artificial Sequence Synthetic primer
caaagacaagcacatcctcgcc

SEQ ID NO: 82 - DNA, 22 bp, Artificial Sequence
  Description of Artificial Sequence Synthetic primer
gccacatagtgcgtgttgagca

SEQ ID NO: 83 - RNA, 43 nt, Homo sapiens
ggcgucccucccgaagcugcgcgcucggucgaacaggacgacc

SEQ ID NO: 84 - RNA, 6 nt, Homo sapiens
ucccga

SEQ ID NO: 85 - RNA, 22 nt, Homo sapiens
ggccgaaaucccgaaugaggcc

SEQ ID NO: 86 - RNA, 21 nt, Homo sapiens
ggaugccucccgagugcaucc

SEQ ID NO: 87 - protein, 446 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNADLILSCCLHYCKDKAKDFMPASKEEPIRLLREVVLLTDDRNLRVKALTRNVPVRDIPAFLTWAQVGGSDYKDHDGDYKDHDIDYKDDDDK

SEQ ID NO: 88 - protein, 435 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGGSGGSGGSGGSGGSGGSMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVGKRPAATKKAGQAKKKKGSDYKDDDDK

SEQ ID NO: 89 - protein, 615 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSATSVDTQRTKGQDNKVQNGSLHQKDTVHDNDFEPYLTGQSNQSNSYPSMSDPYLSSYYPPSIGFPYSLNEAPWSTAGDPPIPYLTTYGQLSNGDHHFMHDAVFGQPGGLGNNIYQHRFNFFPENPAFSAWGTSGSQGQQTQSSAYGSSYTYPPSSLGGTVVDGQPGFHSDTLSKAPGMNSLEQGMVGLKIGDVSSSAVKTVGSVVSSVALTGVLSGNGGTNVNMPVSKPTSWAAIASKPAKPQPKMKTKSGPVMGGGLPPPPIKHNMDIGTWDNKGPVPKAPVPQQAPSPQAAPQPQQVAQPLPAQPPALAQPQYQSPQQPPQTRWVAPRNRNAAFGQSGGAGSDSNSPGNVQPNSAPSVESGSDYKDDDDK

SEQ ID NO: 90 - protein, 341 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 91 - protein, 442 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 92 - protein, 310 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSADFETDESVLMRRQKQINYGKNTIAYDRYIKEVPRHLRQPGIHPKTPNKFKKYSRRSWDQQIKLWKVALHFWDLQLPPLERLTLGGSGGSGGSGGSGGSGGSDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 93 - protein, 411 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSADFETDESVLMRRQKQINYGKNTIAYDRYIKEVPRHLRQPGIHPKTPNKFKKYSRRSWDQQIKLWKVALHFWDLQLPPLERLTLGGSGGSGGSGGSGGSGGSMSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 94 - protein, 327 aa, Artificial Sequence
  Description of Artificial Sequence Synthetic polypeptide
MRVTLSSKPQALATPNKEEHGKRKKKGKGLGKKRDPCLRKYKDFCIHGECKYVKELRAPSCICHPGYHGERCHGLSGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 95 - protein, 428 aa, Artificial Sequence
  Description of Artificial Sequence Synthetic polypeptide
MRVTLSSKPQALATPNKEEHGKRKKKGKGLGKKRDPCLRKYKDFCIHGECKYVKELRAPSCICHPGYHGERCHGLSGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 96 - protein, 297 aa, Artificial Sequence
  Description of Artificial Sequence Synthetic polypeptide
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 97 - protein, 398 aa, Artificial Sequence
  Description of Artificial Sequence Synthetic polypeptide
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 98 - protein, 367 aa, Artificial Sequence
  Description of Artificial Sequence Synthetic polypeptide
MGIINTLQKYYCRVRGGRCAVLSCLPKEEQIGKCSTRGRKCCRRKKGGSGGSGGSGGSGGSGGSADFETDESVLMRRQKQINYGKNTIAYDRYIKEVPRHLRQPGIHPKTPNKFKKYSRRSWDQQIKLWKVALHFWDLQLPPLERLTLGGSGGSGGSGGSGGSGGSMSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 99 - protein, 324 aa, Artificial Sequence
  Description of Artificial Sequence Synthetic polypeptide
MAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVGGSDYKDDDDK

SEQ ID NO: 100 - protein, 432 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVGGSDYKDDDDK

SEQ ID NO: 101 - protein, 423 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVGGSDYKDDDDK

SEQ ID NO: 102 - protein, 430 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLSGSETPGTSESATPESMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVGGSDYKDDDDK

SEQ ID NO: 103 - protein, 496 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGEEEEKKKQQEEEAERLRRIQEEMEKERKRREEDEQRRRKEEEERRMKLEMEAKRKQEEEERKKREDDEKRKKKGSGGSMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVGGSDYKDDDDK

SEQ ID NO: 104 - protein, 339 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLSGSETPGTSESATPESDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 105 - protein, 405 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGEEEEKKKQQEEEAERLRRIQEEMEKERKRREEDEQRRRKEEEERRMKLEMEAKRKQEEEERKKREDDEKRKKKGSGGSDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 106 - protein, 431 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVGSSGHHHHHH

SEQ ID NO: 107 - RNA, 21 nt, Unknown
  Description of Unknown RNA hairpin binding domain
aaacaugaggauuacccaugu

SEQ ID NO: 108 - RNA, 21 nt, Unknown
  Description of Unknown RNA hairpin binding domain
aaacaugaggaucacccaugu

SEQ ID NO: 109 - RNA, 15 nt, Unknown
  Description of Unknown RNA hairpin binding domain
gcccugaagaagggc

SEQ ID NO: 110 - RNA, 15 nt, Unknown
  Description of Unknown RNA hairpin binding domain
gcccugaaaaagggc

SEQ ID NO: 111 - RNA, 25 nt, Pseudomonas aeroginosa
uaaggaguuuauauggaaacccuua

SEQ ID NO: 112 - RNA, 28 nt, Bovine immunodeficiency virus
ggcucguguagcucauuagcuccgagcc

SEQ ID NO: 113 - DNA, 29 bp, Artificial Sequence
  Description of Artificial Sequence Synthetic oligonucleotide
  Description of Combined DNA/RNA Molecule Synthetic oligonucleotide
ggaauccauugcacuccggauuucactag

SEQ ID NO: 114 - RNA, 20 nt, Unknown
  Description of Unknown RNA hairpin binding domain
augcaugucuaagacagcau

SEQ ID NO: 115 - RNA, 23 nt, Unknown
  Description of Unknown RNA hairpin binding domain
aaaacauaaggaaaaccuauguu

SEQ ID NO: 116 - DNA, 20 bp, Artificial Sequence
  Description of Artificial Sequence Synthetic oligonucleotide
cttgtggtagttggagctga

SEQ ID NO: 117 - DNA, 20 bp, Artificial Sequence
  Description of Artificial Sequence Synthetic oligonucleotide
agttggagctgatggcgtag

SEQ ID NO: 118 - DNA, 20 bp, Artificial Sequence
  Description of Artificial Sequence Synthetic oligonucleotide
gttggagctgatggcgtagg

SEQ ID NO: 119 - DNA, 20 bp, Artificial Sequence
  Description of Artificial Sequence Synthetic oligonucleotide
tggcgtaggcaagagtgcct

SEQ ID NO: 120 - DNA, 20 bp, Artificial Sequence
  Description of Artificial Sequence Synthetic oligonucleotide
ggcgtaggcaagagtgcctt

SEQ ID NO: 121 - DNA, 25 bp, Artificial Sequence
  Description of Artificial Sequence Synthetic oligonucleotide
taaggagtttatatggaaaccctta

SEQ ID NO: 122 - DNA, 20 bp, Artificial Sequence
  Description of Artificial Sequence Synthetic oligonucleotide
gtgataagtggaatgccatg

SEQ ID NO: 123 - protein, 403 aa, Artificial Sequence
  Description of Artificial Sequence Synthetic polypeptide
LHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 124 - protein, 13 aa, Artificial Sequence
  Description of Artificial Sequence Synthetic peptide
GSLQLPPLERLTL

SEQ ID NO: 125 - protein, 267 aa, Artificial Sequence
  Description of Artificial Sequence Synthetic polypeptide
MAKTIVLAVGEATRTLTEIQSTADRQIFEEKVGPLVGRLRLTASLRQNGAKTAYRVNLKLDQADVVDASTSVAGELPKVRYTQVWSHDVTIVANSTEASRKSLYDLTKSLVATSQVEDLVVNLVPLGRSLEGGSGGMAKTIVLAVGEATRTLTEIQSTADRQIFEEKVGPLVGRLRLTASLRQNGAKTAYRVNLKLDQADVVDASTSVAGELPKVRYTQVWSHDVTIVANSTEASRKSLYDLTKSLVATSQVEDLVVNLVPLGRSLE

SEQ ID NO: 126 - DNA, 20 bp, Artificial Sequence
  Description of Artificial Sequence Synthetic primer
gaagaaggaaggagcgctaa

SEQ ID NO: 127 - DNA, 20 bp, Artificial Sequence
  Description of Artificial Sequence Synthetic primer
cctgctaccttcatcaccaa

SEQ ID NO: 128 - protein, 613 aa, Artificial Sequence
  Description of Artificial Sequence Synthetic polypeptide
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGGSGGSGGSGGSGGSGGSLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTPKRPAATKKAGQAKKKKGSDYKDDDDK

SEQ ID NO: 129 - protein, 613 aa, Artificial Sequence
  Description of Artificial Sequence Synthetic polypeptide
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGGSGGSGGSGGSGGSGGSLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTPKRPAATKKAGQAKKKKGSDYKDDDDK

SEQ ID NO: 130 - protein, 571 aa, Artificial Sequence
  Description of Artificial Sequence Synthetic polypeptide
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSATSVDTQRTKGQDNKVQNGSLHQKDTVHDNDFEPYLTGQSNQSNSYPSMSDPYLSSYYPPSIGFPYSLNEAPWSTAGDPPIPYLTTYGQLSNGDHHFMHDAVFGQPGGLGNNIYQHRFNFFPENPAFSAWGTSGSQGQQTQSSAYGSSYTYPPSSLGGTVVDGQPGFHSDTLSKAPGMNSLEQGMVGLKIGDVSSSAVKTVGSVVSSVALTGVLSGNGGTNVNMPVSKPTSWAAIASKPAKPQPKMKTKSGPVMGGGLPPPPIKHNMDIGTWDNKGPVPKAPVPQQAPSPQAAPQPQQVAQPLPAQPPALAQPQYQSPQQPPQTRWVAPRNRNAAFGQSGGAGSDSNSPGNVQPNSAPSVESGSDYKDDDDK

SEQ ID NO: 131 - protein, 367 aa, Artificial Sequence; identical to SEQ ID NO: 98
  Description of Artificial Sequence Synthetic polypeptide

SEQ ID NO: 132 - protein, 504 aa, Artificial Sequence
  Description of Artificial Sequence Synthetic polypeptide
MDDPSFLTGRSTYAKRRRARRMNVCKCGAILHNNKDCRSSTISGHKLDRLRFVKEGRVALEGETPVYRTWVKWVETEYHINILETSDDEEGGSGGSGGSGGSGGSGGSMAKTIVLAVGEATRTLTEIQSTADRQIFEEKVGPLVGRLRLTASLRQNGAKTAYRVNLKLDQADVVDASTSVAGELPKVRYTQVWSHDVTIVANSTEASRKSLYDLTKSLVATSQVEDLVVNLVPLGRSLEGGSGGMAKTIVLAVGEATRTLTEIQSTADRQIFEEKVGPLVGRLRLTASLRQNGAKTAYRVNLKLDQADVVDASTSVAGELPKVRYTQVWSHDVTIVANSTEASRKSLYDLTKSLVATSQVEDLVVNLVPLGRSLELQLPPLERLTLGGSGGSGGSGGSGGSGGSDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 133 - protein, 16 aa, Artificial Sequence
  Description of Artificial Sequence Synthetic peptide
SGSETPGTSESATPES

SEQ ID NO: 134 - protein, 73 aa, Artificial Sequence
  Description of Artificial Sequence Synthetic polypeptide
EEEEKKKQQEEEAERLRRIQEEMEKERKRREEDEKRRRKEEEERRMKLEMEAKRKQEEEERKKREDDEKRKKK

SEQ ID NO: 135 - protein, 403 aa, Artificial Sequence
  Description of Artificial Sequence Synthetic polypeptide
LHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 136 - RNA, 30 nt, Unknown
  Description of Unknown RNA hairpin binding domain
  modified_base (1)..(5): a, c, u, g, unknown or other
  modified_base (9)..(13): a, c, u, g, unknown or other
  modified_base (19)..(24): a, c, u, g, unknown or other
  modified_base (26)..(30): a, c, u, g, unknown or other
nnnnnugcnnnnncagugnnnnnncnnnnn

SEQ ID NO: 137 - protein, 4 aa, Homo sapiens
DEAH

SEQ ID NO: 138 - protein, 4 aa, Homo sapiens
DEAD

SEQ ID NO: 139 - RNA, 68 nt, Artificial Sequence
  Description of Artificial Sequence Synthetic oligonucleotide
  modified_base (32)..(68): a, c, u, g, unknown or other
ggccagaucugagccugggagcucucuggccnnnnnnnnnnnnnnnnnnnnnnnnnnnnnnnnnnnnn

SEQ ID NO: 140 - DNA, 63 bp, Artificial Sequence
  Description of Artificial Sequence Synthetic oligonucleotide
  Description of Combined DNA/RNA Molecule Synthetic oligonucleotide
  modified_base (32)..(63): a, c, t, g, unknown or other
ccaaaggcucuucucagagccacccattattnnnnnnnnnnnnnnnnnnnnnnnnnnnnnnnn

SEQ ID NO: 141 - RNA, 63 nt, Artificial Sequence
  Description of Artificial Sequence Synthetic oligonucleotide
  modified_base (32)..(63): a, c, u, g, unknown or other
ccaaaggcucuucucagagccacccauuauunnnnnnnnnnnnnnnnnnnnnnnnnnnnnnnn

SEQ ID NO: 142 - DNA, 27 bp, Artificial Sequence
  Description of Artificial Sequence Synthetic oligonucleotide
  modified_base (13)..(15): a, c, t, g, unknown or other
gtagttggagctnnnggcgtaggcaag

SEQ ID NO: 143 - DNA, 20 bp, Artificial Sequence
  Description of Artificial Sequence Synthetic oligonucleotide
tgaacaccatcaacctcgac

SEQ ID NO: 144 - DNA, 20 bp, Artificial Sequence
  Description of Artificial Sequence Synthetic oligonucleotide
gaacaccatcaacctcgact

SEQ ID NO: 145 - DNA, 20 bp, Artificial Sequence
  Description of Artificial Sequence Synthetic oligonucleotide
tcaacctcgactaccgcatc

SEQ ID NO: 146 - DNA, 20 bp, Artificial Sequence
  Description of Artificial Sequence Synthetic oligonucleotide
caacctcgactaccgcatcc

SEQ ID NO: 147 - DNA, 20 bp, Artificial Sequence
  Description of Artificial Sequence Synthetic oligonucleotide
accgcatccgttctcacgga

SEQ ID NO: 148 - DNA, 20 bp, Artificial Sequence
  Description of Artificial Sequence Synthetic oligonucleotide
ccgcatccgttctcacggaa

(19) United States
(12) Patent Application Publication
(10) Pub. No.: US 2022/0048962 A1
(43) Pub. Date: Feb. 17, 2022

(54) SYSTEMS AND METHODS FOR MODULATING RNA

(71) Applicant: The University of Chicago, Chicago, IL (US)

(72) Inventors: Bryan C. DICKINSON, Chicago, IL (US); Simone RAUCH, Chicago, IL (US)

(73) Assignee: The University of Chicago, Chicago, IL (US)

(21) Appl. No.: 17/309,936

(22) PCT Filed: Jan. 3, 2020
(86) PCT No.: PCT/US2020/012169

§ 371 Date: Jul. 1, 2021

Related U.S. Application Data
(60) Provisional application No. 62/788,571, filed on Jan. 4, 2019.
(60) Provisional application No. 62/831,342, filed on Apr. 9, 2019.
(60) Provisional application No. 62/903,080, filed on Sep. 20, 2019.
(60) Provisional application No. 62/929,339, filed on Nov. 1, 2019.

(51) Int. Cl. C07K 14/47; C12N 15/86; C12N 15/11; C12N 9/22

(52) U.S. Cl. CPC C07K 14/4702; C12N 15/86; A61K 38/00; C12N 9/22; C07K 2319/85; C12N 15/11

(57) ABSTRACT

Aspects of the disclosure relate to a RNA regulatory system comprising at least one of each: i) a RNA hairpin binding domain; ii) a RNA targeting molecule comprising a RNA targeting region and at least one hairpin structure, wherein the hairpin structure of the RNA targeting molecule specifically binds to i; and iii) a RNA regulatory domain.

### CROSS-REFERENCE TO RELATED APPLICATIONS

This application claims the benefit of priority of U.S. Provisional Patent Application No. 62/788,571 filed Jan. 4, 2019, U.S. Provisional Patent Application No. 62/831,342 filed Apr. 9, 2019, U.S. Provisional Patent Application No. 62/903,080 filed Sep. 20, 2019, and U.S. Provisional Patent Application No. 62/929,339 filed Nov. 1, 2019, all of which are hereby incorporated by reference in their entirety.

### STATEMENT OF GOVERNMENT SUPPORT

This invention was made with government support under GM119840 and HG008935 awarded by The National Institutes of Health. The government has certain rights in the invention.

### BACKGROUND OF THE INVENTION

### 1. Field of the Invention

The present invention relates generally to the field of chemistry and medicine. More particularly, it concerns the use of a system for modulating RNA.

### 2. Description of Related Art

Programmable nucleic acid-binding proteins have revolutionized genome studies and editing technologies (Chandrasegaran and Carroll, 2016; Filipovska et al., 2011; Gootenberg et al., 2018; Hilton et al., 2015; Joung and Sander, 2012; Kearns et al., 2015; Strutt et al., 2018) and are opening up new therapeutic opportunities to treat human diseases (Liao et al., 2017; Monteys et al., 2017). In particular, the CRISPR/Cas9 system, which evolved as a bacterial immune defense mechanism, has transformed the ability to study and manipulate cellular DNA site-specifically (Cong et al., 2013; Jiang et al., 2013; O'Connell et al., 2014; Wiedenheft et al., 2012). A key advantage of CRISPR/Cas systems compared to previous methods (Desjarlais and Berg, 1993; Hockemeyer et al., 2011; Joung and Sander, 2012; Schierling et al., 2012) is that they are easily programmable to target virtually any locus of interest. The CRISPR/Cas system is a ribonucleoprotein complex that uses base pair interactions of a displayed guide RNA (gRNA) to interact with a target nucleic acid sequence. The simple nature of base pair-guided targeting opens up the possibility to program systems to interact with a defined nucleic acid sequence by simply changing the nucleic acid sequence on the guiding strand.

While targeting DNA directly will have profound clinical ramifications, diseases that involve subtle alterations to many genes will be challenging to target using DNA editing technologies (Fuxman Bass et al., 2015). Additionally, the potential side effects or risks of permanent genetic alteration might not be tolerated. For example, the genes one may want to target to activate an enhanced wound healing response are likely targets that could pose a risk for cancer development, making permanent DNA-based strategy risky. Targeting information flow at the RNA level presents several opportunities for therapeutic intervention, including but not limited to the ability to halt treatment if side effects emerge, the ability to target genes that would be too risky to alter at the DNA level, and the ability to manipulate gene expression without permanent alterations to the host genome. While inhibiting or enhancing transcription at the genome level provides one possibility for controlling gene expression (Du et al., 2017; Fuxman Bass et al., 2015; Qi et al., 2013), recently discovered RNA epitranscriptomic regulatory mechanisms offer a broad range of RNA regulatory processes to target, including editing, degradation, transport, and translation of RNA transcripts (Nishikura, 2010; Roundtree et al., 2017; Zhao et al., 2017). Although the mechanisms and consequences of this epitranscriptomic regulatory layer are just beginning to be uncovered, it is apparent that the information flow through RNA is tightly regulated, offering many new opportunities for both basic research discoveries as well as therapeutic development.

Programmable RNA-targeting tools analogous to the dCas9 DNA-targeting systems hold great promise for studying the mechanisms of epitranscriptomic regulation and for therapeutic applications. The current tools for RNA targeting involve the delivery of large complexes and pose immunogenicity issues. From a basic science perspective, the large size of the delivery vehicle could lead to potential perturbations to the RNA under interrogation, convoluting the study of RNA regulatory mechanisms. From a translational perspective, the large size presents challenges for viral packaging or direct protein delivery. Additionally, while DNA-editing therapies will likely consist of a one-time, irreversible treatment, RNA-targeting therapies will need to be continually administered, making delivery concerns especially important. Moreover, it was recently discovered that 85% of people already have circulating antibodies to CRISPR/Cas proteins (Kim et al., 2018; Wagner et al., 2018), suggesting immunogenicity issues may prove problematic in clinical applications. Therefore, there is a need in the art for improved systems that can target RNA and be delivered efficiently without activating an immune response.

### SUMMARY OF THE INVENTION

To overcome the large size and microbial-derived nature of current RNA-targeting systems, the inventors present a CRISPR/Cas-inspired RNA targeting system (CIRTS), a general method for engineering programmable RNA effector proteins. Similar to CRISPR/Cas-based systems, CIRTS is a ribonucleoprotein complex that uses Watson-Crick-Franklin base pair interactions to deliver protein cargo site-selectively in the transcriptome. The inventors show they can easily engineer CIRTS that deliver a range or regulatory proteins to transcripts, including nucleases for degradation, deadenylation regulatory machinery for degradation, or translational activation machinery for enhanced protein production. However, CIRTS are up to 5-fold smaller than the smallest current CRISPR/Cas systems and can be engineered entirely from human parts.

Aspects of the disclosure relate to a RNA regulatory system or method comprising at least one of each: i) a RNA hairpin binding domain; ii) a RNA targeting molecule comprising a RNA targeting region and at least one hairpin structure, wherein the hairpin structure of the RNA targeting molecule specifically binds to i; and iii) a RNA regulatory domain. In some embodiments, the following are included: i) and ii), i) and iii), ii) and iii), or i), ii), and iii). Any embodiment disclosed herein can contain any of these combinations.

Further aspects relate to a vector system comprising one or more nucleic acid vectors comprising a nucleotide encoding: i) a RNA hairpin binding domain; ii) a RNA targeting molecule comprising a RNA targeting region and at least one hairpin structure, wherein the hairpin structure of the RNA targeting molecule specifically binds to i), and iii) a RNA regulatory domain.

Further aspects relate to a fusion protein comprising a RNA hairpin binding protein and a RNA regulatory domain and nucleic acids encoding such fusion proteins.

Further aspects relate to a conjugate comprising a RNA regulatory domain operably linked to a RNA targeting molecule, wherein the RNA targeting molecule comprises a RNA targeting region and at least one hairpin structure. In some embodiments, the RNA regulatory domain and the RNA targeting molecule are operably linked through a peptide bond. In some embodiments, the polypeptide further comprises one or more linkers. In some embodiments, the RNA regulatory domain and the RNA targeting molecule are operably linked through non-covalent interactions. In some embodiments, the RNA regulatory domain is covalently linked to a first dimerization domain and the RNA targeting molecule is covalently linked to a second dimerization domain and wherein the first and second dimerization domain are capable of dimerizing to form a non-covalent or covalent linkage. In some embodiments, the conjugate comprises one or more nuclear localization signals (NLS)s.

Yet further aspects relate to a delivery vehicle comprising a system of the disclosure. In some embodiments, the delivery vehicle comprises liposome(s), particle(s), exosome(s), microvesicle(s), a gene-gun or one or more nucleic acid vector(s).

Further aspects relate to a composition or a cell comprising a system, delivery vehicle, or fusion protein of the disclosure.

Further aspects relate to a method of modulating at least one target RNA comprising contacting the target RNA with a system, composition, or fusion protein of the disclosure. In some embodiments, modulating at least one target RNA comprises cleaving, demethylating, methylating, activating translation, repressing translation, promoting degradation, or binding to the RNA. In some embodiments, at least two target RNAs are modulating. In some embodiments, at least 2, 3, 4, 5, 6, 7, 8, 9, 10, 15, 20, or 25 (or any derivable range therein) target RNAs are modulated. In some embodiments, the multiple RNAs are modulated by the same RNA regulatory domain or by a regulatory domain with the same activity. In some embodiments, the different target RNAs are modulated with a different activity, such as by cleaving, demethylating, methylating, activating translation, repressing translation, promoting degradation, or binding to the RNA.

In some embodiments, the RNA regulatory domain does not bind to RNA. In some embodiments, the RNA regulatory domain comprises a polypeptide that does not have RNA binding activity. In some embodiments, the RNA regulatory domain does not bind to modified RNA. In some embodiments, the RNA regulatory domain does not bind to m6A modified RNA.

Further aspects relate to a cell or progeny thereof comprising modulated target RNA, wherein the target RNA has been modulated according to a method of the disclosure. Further aspects relate to a multicellular organism comprising one or more cells of the disclosure. Further aspects relate to a plant or animal comprising one or more cells of the disclosure.

Further aspects relate to a kit comprising a system, vector, delivery vehicle, or fusion protein of the disclosure.

Further aspects relate to a method for modulating a target RNA in a subject, the method comprising administering a system or composition of the disclosure to the subject.

The term “RNA hairpin” refers to a RNA molecule with stem-loop intramolecular base pairing. A hairpin can occur when two regions of the same strand, usually complementary in nucleotide sequence when read in opposite directions, base-pair to form a double helix that ends in an unpaired loop. The disclosure relates to engineered RNA targeting molecules comprising a RNA targeting region and one or more hairpins. Accordingly, the engineered RNA molecules of the disclosure are chimeric molecules that are non-naturally occurring.

The term “RNA targeting region” refers to a region of the RNA that is capable of hybridizing to a target RNA. The target RNA may be a disease associated RNA or one that is a modulation target according to the current systems and methods.

The “RNA regulatory domain” refers to a peptide or polypeptide that has activity directed to RNA. Examples of activity include methylation activity, RNA-binding activity, nuclease activity, and translational activation or repression activity. Further examples of activities and proteins comprising RNA regulatory domains are described throughout the disclosure.

In some embodiments, the RNA hairpin binding domain and the RNA regulatory domain are operably linked. The term “operably linked” refers to two proteins that are linked through either covalent or non-covalent interactions. For example, the two proteins may be covalently linked through a peptide bond. In some embodiments, the proteins are non-covalently linked. One or more proteins of the disclosure may be operably linked to another protein through linkage to a pair of accessory proteins that have a strong affinity for each other. Such accessory proteins are known in the art. For example, the SunTag is one such system that includes an antibody with a strong affinity for a peptide. One protein, polypeptide, or domain of the disclosure may be linked to a SunTag peptide and another protein, polypeptide, or domain of the disclosure may be linked to an antibody to allow operable linkage of the two proteins, polypeptides, or domains through the interaction of the SunTag peptide and antibody. Further examples include biotin and avidin/streptavidin and spytag and spycatcher.

In some embodiments, the system is inducible by providing the RNA regulatory domain and the hairpin binding domain as two unlinked polypeptides that become linked upon the presence of a stimulant. The induction may be, for example, by light induction or by chemical induction. Such inducibility allows for activation of the RNA regulation at a desired moment in time. In some embodiments, the RNA regulatory domain is covalently linked to a first dimerization domain and the RNA hairpin binding domain is covalently linked to a second dimerization domain and wherein the first and second dimerization domain are capable of dimerizing to form a non-covalent or covalent linkage. In some embodiments, the dimerization is inducible. In some aspects, the dimerization is induced through binding of the dimerization domains to a ligand. The term inducible refers to dimerization that is formed in response to a stimulus, such as a ligand, a chemical, a temperature change, or light, for example.

Light inducibility is for instance achieved by designing a fusion complex wherein the first and second dimerization domains comprise CRY2PHR and CIBN. This system is particularly useful for light induction of protein interactions in living cells and is further described in Konermann S, et al. Nature. 2013;500:472-476, which is herein incorporated by reference.

Suitable dimerization domains and corresponding ligands are known in the art. For example, Liang, F. S., Ho, W. Q., and Crabtree, G. R. (2011). Engineering the ABA plant stress pathway for regulation of induced proximity. Sci. Signal. 4, rs2, which is incorporated by reference, describes suitable dimerization/ligand systems that are useful in embodiments of the disclosure. In some embodiments, one of the first or second dimerization domain comprises PYR/PYR1-like (PYL1), the other of the first or second domain comprises ABA insensitive 1 (ABI1), and the ligand comprises abscisic acid (ABA) or derivatives or fragments thereof. The dimerization domain may be a fragment or portion of the whole protein and may be a substituted or modified. In some embodiments, the first and/or second dimerization domain comprises FKBP12 and the ligand comprises FK1012 or derivatives or fragments thereof. In some embodiments, one of the first or second dimerization domain comprises FK506 binding protein (FKBP), the other of the first or second domain comprises FKBP-Rap binding domain of mammalian target of Rap mTOR (Frb), and the ligand comprises rapamycin (Rap) or derivatives or fragments thereof.

Derivatives refer to modified ligands and domains that retain binding or have enhanced binding to their dimerization domain or ligand, respectively. Fragments refer to contiguous portions of the dimerization domains that retain binding to the ligand. In some embodiments, the dimerization domain may be a modified fragment.

In some embodiments, i, ii, and/or iii are human or are human-derived. In some embodiments, the system, conjugate, and/or fusion protein is non-immunogenic. A human protein, polypeptide, domain, or nucleic acid refers to a protein, polypeptide, domain, or nucleic acid that is from the human genome, although it may be produced recombinantly in non-human systems. The term “human-derived” refers to a protein, polypeptide, domain, or nucleic acid that is a variant or fragment of a protein, polypeptide, domain, or nucleic acid from the human genome, although it may be produced recombinantly in non-human systems. In some embodiments, the fusion protein, conjugate, system, or parts thereof, such as parts i, and/or iii are non-immunogenic and/or non-toxic when expressed in or administered to humans.

In some embodiments, the nucleic acids or polypeptides of the disclosure are synthetic, are non-natural, and/or do not occur naturally in nature.

In some embodiments, the system further comprises a stabilizer polypeptide; wherein the stabilizer polypeptide comprises a cationic polypeptide that binds non-specifically to nucleic acids. In some embodiments, the stabilizer polypeptide is human-derived. In some embodiments, the stabilizer polypeptide is operably linked to the RNA regulatory domain and/or RNA hairpin binding domain. In some embodiments, the stabilizer polypeptide comprises ORF5 or a fragment thereof. In some embodiments, the stabilizer polypeptide comprises SEQ ID NO:5, a variant thereof, or a polypeptide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, or 99% (or any derivable range therein) identity or homology to SEQ ID NO:5. In some embodiments, the stabilizer polypeptide comprises HEBGF or a fragment thereof. In some embodiments, the stabilizer polypeptide comprises SEQ ID NO:19, a variant thereof, or a polypeptide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, or 99% (or any derivable range therein) identity or homology to SEQ ID NO:19. In some embodiments, the stabilizer polypeptide comprises β-defensin 3 or a fragment thereof. In some embodiments, the stabilizer polypeptide comprises SEQ ID NO:20, a variant thereof, or a polypeptide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, or 99% (or any derivable range therein) identity or homology to SEQ ID NO:20.

In some embodiments, the stabilizer polypeptide, conjugate, fusion protein, conjugate, RNA regulatory domain, and/or RNA hairpin binding domain are less than, more than, or are at most or at least 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, 20, 15, 10, or 5 kDa (or any derivable range therein). In some embodiments, the total complex comprising the RNA regulatory domain and hairpin binding domain is less than, more than, or is at most or at least 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, 20, 15, 10, or 5 kDa (or any derivable range therein). In some embodiments, the total complex comprising the stabilizer polypeptide, RNA regulatory domain and hairpin binding domain is less than, more than, or is at most or at least 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, 20, 15, 10, or 5 kDa (or any derivable range therein).

In some embodiments, the RNA hairpin binding domain comprises a RNA hairpin binding domain from U1A (TBP6.7), SLBP, or variants thereof. In some embodiments, the RNA hairpin binding domain comprises a RNA hairpin binding domain from U1A (TBP6.7), SLBP, Ku70, nucleolin, or variants thereof. In some embodiments, the RNA hairpin binding domain comprises SEQ ID NO:7 or 18, a variant thereof, or a polypeptide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, or 99% (or any derivable range therein) identity or homology to SEQ ID NO:7 or 18.

In some embodiments, the RNA targeting molecule comprises a TAR hairpin scaffold. In some embodiments, the RNA targeting molecule comprises the TAR hairpin scaffold of SEQ ID NO:1 or a nucleotide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, or 99% (or any derivable range therein) identity to SEQ ID NO:1. In some embodiments, the RNA targeting molecule comprises a SLBP hairpin scaffold. In some embodiments, the RNA targeting molecule comprises the SLBP hairpin scaffold of SEQ ID NO:2 or a nucleotide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, or 99% (or any derivable range therein) identity to SEQ ID NO:2.

In some embodiments, the RNA targeting molecule comprises exactly one hairpin. In some embodiments, the RNA targeting molecule comprises at least one hairpin. In some embodiments, the RNA targeting molecule comprises exactly two hairpins. In some embodiments, the RNA targeting molecule comprises at least two hairpins. In some embodiments, the RNA targeting molecule comprises exactly three hairpins. In some embodiments, the RNA targeting molecule comprises at least three hairpins. In some embodiments, the RNA targeting molecule comprises exactly four hairpins. In some embodiments, the RNA targeting molecule comprises at least four hairpins. In some embodiments, the RNA targeting molecule comprises exactly five hairpins. In some embodiments, the RNA targeting molecule comprises at least five hairpins. In some embodiments, the RNA targeting molecule comprises 1-4 hairpins. In some embodiments, the RNA targeting molecule comprises 1-3 hairpins. In some embodiments, the RNA targeting molecule comprises 1-2 hairpins. In some embodiments, the RNA targeting molecule comprises 2-4 hairpins. In some embodiments, the RNA targeting molecule comprises 2-3 hairpins. In some embodiments, the RNA targeting molecule comprises at least, at most, or exactly 1, 2, 3, 4, 5, or 6 hairpins (or any range derivable therein). In some embodiments, the RNA targeting molecule comprises at least one hairpin that does not bind to the RNA hairpin binding protein and at least one hairpin that binds to the RNA hairpin binding protein. In some embodiments, the RNA targeting molecule binds to more than one RNA binding protein. In some embodiments, the RNA targeting molecule comprises two, three, or four hairpin structures and binds to at least two RNA binding proteins. In some embodiments, the RNA regulatory system comprises at least two regulatory domains, wherein each regulatory domain binds to a different RNA binding molecule.

In some embodiments, the RNA targeting molecule comprises one or more modified nucleotides. In some embodiments, the modified nucleotides comprise a modification such as a phosphorothioate, locked nucleotides, ethylene bridged nucleotides, peptide nucleic acids, 5′E-VP, or is modified to a morpholino. In some embodiments, the modification includes one described herein.

In some embodiments, the RNA hairpin binding domain comprises the RNA hairpin binding domain of U1A, a variant thereof, or a polypeptide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, or 99% (or any derivable range therein) identity or homology to SEQ ID NO:7 and the RNA targeting molecule comprises a TAR hairpin scaffold or a nucleotide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, 99, or 100% (or any derivable range therein) identity to SEQ ID NO:1.

In some embodiments, the RNA hairpin binding domain comprises the RNA hairpin binding domain of SLBP, a variant thereof, or a polypeptide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, or 99% (or any derivable range therein) identity or homology to SEQ ID NO:18 and the RNA targeting molecule comprises a SLBP hairpin scaffold or a nucleotide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, 99, or 100% (or any derivable range therein) identity to SEQ ID NO:2.

In some embodiments, the RNA hairpin binding domain comprises the RNA hairpin binding domain of ku70 or a variant thereof, and the RNA targeting molecule comprises a hairpin scaffold or a nucleotide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, 99, or 100% (or any derivable range therein) identity to SEQ ID NO:83.

In some embodiments, the RNA hairpin binding domain comprises the RNA hairpin binding domain of nucleolin or a variant thereof, and the RNA targeting molecule comprises a hairpin scaffold or a nucleotide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, 99, or 100% (or any derivable range therein) identity to one of SEQ ID NO:84-86.

In some embodiments, the RNA hairpin binding domain, stabilizer polypeptide, or RNA hairpin binding domain comprises a linker. In some embodiments, the linker comprises a polypeptide comprising SEQ ID NO:6, 21, 22, 23, or 25 or a polypeptide with at least 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, or 99% (or any derivable range therein) identity or homology to SEQ ID NO:6, 21, 22, 23, or 25. In some embodiments, the linker is a rigid linker. In some embodiments, the linker is a flexible linker. In some embodiments, the linker comprises glycine and serine residues. In some embodiments, the linker is at least 4 amino acids. In some embodiments, the linker is at least or at most or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 44, 45, 46, 47, 48, 49, or 50 amino acids (or any derivable range therein).

In some embodiments, the stabilizer polypeptide comprises a polypeptide, such as a RNA-binding polypeptide, from cJun, HBEGF, HRX, NDEK, NHGF, beta-defensin3, or scGFP. In some embodiments, the RNA regulatory domain is operably linked to the stabilizer polypeptide at the carboxy terminus of the RNA regulatory domain. In some embodiments, the RNA regulatory domain is operably linked to the stabilizer polypeptide at the amino terminus of the RNA regulatory domain. In some embodiments, the RNA regulatory domain is operably linked to the RNA hairpin binding domain polypeptide at the carboxy terminus of the RNA regulatory domain. In some embodiments, the RNA regulatory domain is operably linked to the RNA hairpin binding domain polypeptide at the amino terminus of the RNA regulatory domain. In some embodiments, the RNA hairpin binding domain polypeptide is operably linked to the stabilizer polypeptide at the carboxy terminus of the RNA hairpin binding domain polypeptide. In some embodiments, the RNA hairpin binding domain polypeptide is operably linked to the stabilizer polypeptide at the amino terminus of the RNA hairpin binding domain polypeptide.

In some embodiments, the RNA targeting region comprises at least 12 nucleotides. In some embodiments, the RNA targeting region comprises at least, at most, or exactly 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 79 nucleotides (or any derivable range therein).

In some embodiments, the RNA regulatory domain comprises a nuclease, methylase, demethylase, translational activator, translational repressor, single-stranded RNA cleavage activity, double-stranded RNA cleavage activity, or RNA binding activity. In some embodiments, the RNA regulatory domain comprises an activity described herein.

In some embodiments, the RNA regulatory domain comprises a Pin nuclease domain or a m6A reader protein or portion thereof. In some embodiments, the RNA regulatory domain comprises a domain or polypeptide from SMG6, YTHDF1, or YTHDF2. In some embodiments, the RNA regulatory domain comprises a domain or polypeptide from an ADAR protein. In some embodiments, the RNA regulatory domain comprises a domain or polypeptide from a human ADAR protein. In some embodiments, the RNA regulatory domain comprises a polypeptide that has at least, at most, or exactly 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, 99, or 100% (or any derivable range therein) identity or homology to SEQ ID NO:9, 11, 15, 16, 17, or 123-125. In some embodiments, the RNA regulatory domain further comprises a helical region. In some embodiments, the helical region comprises a polypeptide that has at least, at most, or exactly 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, 99, or 100% (or any derivable range therein) identity or homology to SEQ ID NO:24.

In some embodiments, the RNA regulatory domain increases translation of a target RNA. In some embodiments, the RNA regulatory domain increases degradation of a target RNA. In some embodiments, the RNA regulatory domain modifies the localization of a target RNA. In some embodiments, the RNA regulatory domain modifies the processing of the target RNA.

In some embodiments, the RNA regulatory domain comprises a polypeptide, such as a polypeptide having RNA regulatory activity from IFIT2, eIF4a, eIF4e, PABP, PAIP, SLBP, BOLL, ICP27, YTHDF1, YTHDF2, or YTHDF3. In some embodiments, the RNA regulatory domain comprises a polypeptide, such as a polypeptide having RNA regulatory activity from YTHDF2, TOB2, ZFP36, CNOT7, RNaseA, RNaseL, RNaseP, RNase4, RNase1, RNaseU2, or HRSP12. In some embodiments, the RNA regulatory domain increases the expression of a polypeptide encoded by the target RNA and wherein the RNA regulatory domain comprises IFIT2, eIF4a, eIF4e, PABP, PAIP, SLBP, BOLL, ICP27, YTHDF1, or YTHDF3. In some embodiments, the RNA regulatory domain comprises a polypeptide, such as a polypeptide having RNA regulatory activity from YTHDF2, TOB2, ZFP36, CNOT7, RNaseA, RNaseL, RNaseP, RNase4, RNase1, RNaseU2, or HRSP12. In some embodiments, the RNA regulatory domain decreases the expression of a polypeptide encoded by the target RNA and wherein the RNA regulatory domain comprises YTHDF2, TOB2, ZFP36, CNOT7, RNaseA, RNaseL, RNaseP, RNase4, RNase1, RNaseU2, or HRSP12.

In some embodiments, one or more nuclear export signals (NES) are fused to the RNA regulatory domain, the RNA hairpin binding domain, and/or the stabilizing polypeptide. In some embodiments, the NES is at the carboxy terminus of the RNA regulatory domain, the RNA hairpin binding domain, and/or the stabilizing polypeptide. In some embodiments, the NES is at the amino terminus of the RNA regulatory domain, the RNA hairpin binding domain, and/or the stabilizing polypeptide. In some embodiments, the NES comprises a polypeptide that has at least, at most, or exactly 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, 99, or 100% (or any derivable range therein) identity or homology to SEQ ID NO:8.

In some embodiments, one or more nuclear localization signals (NLS) are fused to the RNA regulatory domain, the RNA hairpin binding domain, and/or the stabilizing polypeptide. In some embodiments, the NLS is at the carboxy terminus of the RNA regulatory domain, the RNA hairpin binding domain, and/or the stabilizing polypeptide. In some embodiments, the NLS is at the amino terminus of the RNA regulatory domain, the RNA hairpin binding domain, and/or the stabilizing polypeptide. In some embodiments, the NES comprises a polypeptide that has at least, at most, or exactly 60, 61, 62, 63, 64, 65, 66, 67, 68, 69, 70, 75, 76, 77, 78, 79, 80, 85, 86, 87, 88, 89, 90, 91, 92, 93, 94, 95, 96, 97, 98, 99, or 100% (or any derivable range therein) identity or homology to SEQ ID NO:13.

In some embodiments, the RNA targeting region of ii hybridizes to a target RNA in a prokaryotic or eukaryotic cell. In some embodiments, the target RNA is in a human cell. In some embodiments, the target RNA is in vitro or in vivo.

In some embodiments, the system comprises at least two of each i, ii, and iii. In some embodiments, the at least two of i, ii, and iii are expressed in the same cell. In some embodiments, the method comprises modulating at least two target RNAs. In some embodiments, the system comprises at least, at most, or exactly 2, 3, 4, 5, 6, 7, 8, 9, 10, 20, 30, 40, or 50 or more (or any derivable range therein) of i, ii, and iii. In some embodiments, at least, at most, or exactly 2, 3, 4, 5, 6, 7, 8, 9, 10, 20, 30, 40, or 50 or more (or any derivable range therein) target RNAs are modulated in a cell.

In some embodiments, the RNA regulatory domain cleaves RNA, promotes RNA translation, inhibits RNA translation, or modifies the base sequence of RNA.

In some embodiments, the vectors of the disclosure further comprise a regulatory element operably linked to the nucleotide encoding i, ii, and/or iii. Regulatory elements, in addition to a NLS and NES, as previously described, also include promoters, polyadenylation signals, enhancers, etc. Other regulatory elements are known in the art and described herein and may be used in the embodiments of the disclosure. In some embodiments, the one or more nucleic acid vectors are optimized for expression in an eukaryotic cell. In some embodiments, the expression of the domains, RNA, or polypeptides in the cell or from a vector is constitutive. In some embodiments, the expression of the domains, RNA, or polypeptides in the cell or from a vector is conditional. In some embodiments, i, ii, and iii are on a single vector. In some embodiments, i, ii, iii, and the stabilizer polypeptide are encoded on a single vector. In some embodiments, i, iii, and the stabilizer polypeptide are encoded on a single vector. In some embodiments, one or more of the vectors are viral vectors. In some embodiments, the one or more vectors comprise one or more retroviral, lentiviral, adenoviral, adeno-associated or herpes simplex viral vectors. In some embodiments, one or more of the vectors are non-viral vectors. In some embodiments, the system or composition is non-viral, which denotes that it does not contain any viral components.

In some embodiments, there is a system or kit comprising one or more of the following components: a polypeptide comprising a RNA regulatory domain, a polypeptide comprising a RNA binding domain, a polypeptide comprising a stabilizer, a nucleic acid encoding for a RNA regulatory domain, a nucleic acid encoding for a RNA binding domain, a nucleic acid encoding a stabilizer, a nucleic acid encoding a RNA targeting molecule comprising a RNA targeting region and at least one hairpin structure; a conjugate of the disclosure; a vector of the disclosure, a fusion protein of the disclosure, a recombinant host cell, an expression construct, an engineered viral vector, or an engineered attenuated virus. In certain embodiments, a polypeptide of the disclosure is under the control of a heterologous promoter. It is specifically contemplated that any protein or polypeptide function that are used in embodiments, may be used a nucleic acid encoding that protein or polypeptide function. Also, any and all polypeptides, proteins, nucleic acid molecules may be contained within a cell or other living organism, such as a virus (for instance, a phage).

A kit may include one or more components that are separate or together in a suitable container means, such as a sterile, non-reactive container. In some embodiments, cells or viruses are provided that contain one or more nucleic acid constructs that encode the polypeptides of the disclosure. The term “promoter” is used according to its ordinary meaning to those in the field of molecular biology; it generally refers to a site on a nucleic acid in which a polymerase can bind to initiate transcription. In specific embodiments, the promoter is recognized by a T7 RNA polymerase.

The compositions, vectors, systems, methods, and proteins of the disclosure are useful for a variety of clinical and research-related applications. The embodiments of the disclosure may be useful for the treatment of a disease or condition, such as cancer or autoimmunity. In some embodiments, the methods and compositions are for the acute treatment of a disease or condition. In some embodiments, the methods and compositions are useful for the temporary modulation of RNA. In some embodiments exclude permanent modification of gene activity. In some embodiments, the methods and compositions are safer due to the acute modulation of RNA and/or due to the ability to control the expression of the system in vivo.

As used herein the specification, “a” or “an” may mean one or more. As used herein in the claim(s), when used in conjunction with the word “comprising”, the words “a” or “an” may mean one or more than one.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and Description of Figure Legends.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-D. Design of CRISPR/Cas-inspired RNA targeting system (CIRTS) (A) Schematic overview of the design strategy. CIRTS is composed of a ssRNA binding protein an RNA hairpin binding protein, an effector protein and a guiding RNA. (B) List of modular CIRTS constructs used in this work. (C) Design of the guiding RNA for TBP6.7. The HIV TAR hairpin was fused to a nucleotide linker (L)and a guide sequence. (D) Design of the guiding RNA for the RRM of SLBP. The human histone mRNA hairpin was fused to a flexible five nucleotide linker and a guide sequence.

FIG. 2A-C. CIRTS-1 in vitro binding and RNA cleavage assays (A)

Electrophoretic mobility assay (EMSA) evaluating the binding affinity of MBP-CIRTS-1:on-target gRNA (R3) complex to a labeled RNA substrate (R1). EDTA was supplemented to the reaction buffer to avoid any cleavage. (B) Calculation of the binding affinity by fitting the fraction of TBP6.7:gRNA bound to substrate to a quadratic binding equation. (C) Cleavage assay run on a denaturing gel after 2 h of incubation. A labeled RNA substrate (R2) is cleavage in a gRNA-dependent manner.

FIG. 3A-G. CIRTS mammalian cell reporter assays (A) General overview of the dual luciferase assay. A reporter construct that contains both firefly luciferase and Renilla luciferase is used in all assays. The inventors targeted their CIRTS to the firefly luciferase transcript, while keeping Renilla luciferase constant to use as a transfection control. For all subsequent assays, HEK293T cells were transfected with the reporter vector, the CIRTS vector, and a gRNA vector. (B) Catalytically-inactive CIRTS-0 was used as a control. After 48 h of incubation, the inventors observed no decrease in protein readout. (C) Comparison of CIRTS-1 with Cas13b nuclease. Cells transfected with either CIRTS-1 or Cas13 and the corresponding gRNA targeting show reduced protein levels after incubation. (D) HEK293T cells transfected with CIRTS-2 show an increase in protein level after 48 h, (E) cells transfected with CIRTS-3, however, show the anticipated decrease in protein level. (F) Switching the hairpin-binding protein to SLBP still results in decrease protein levels after 48 h. (G) Cells transfected with a fully humanized CIRTS (CIRTS-5 and CIRTS-6) system and an on-target gRNA for firefly luciferase result again in decreased protein levels.

FIG. 4A-C. Targeting endogenous transcripts with CIRTS (A) Nuclease-mediated knockdown of five endogenous transcripts upon transfection of cells with CIRTS-1 as assayed using qPCR. CIRTS-1 can be used to target endogenous transcripts of interest by co-transfecting a gRNA the corresponding on-target guiding sequence. (B) qPCR analysis of non-nuclease-mediated knockdown of endogenous transcripts with CIRTS-3. Cells transfected with CIRTS-3 show gRNA-dependent decreases in RNA level for all five transcripts tested. (C) Analysis of protein levels after transfection with CIRTS-2 or CIRTS-3 using Western blot. CIRTS-2 can induce an increase in protein levels, whereas CIRTS-3 shows the expected decrease in protein levels as a control.

FIG. 5A-B. Multidimensional targeting with CIRTS. (A) Schematic of vectors used for multiplexed targeting. CIRTS-6 was co-transfected with its gRNA construct targeting PPIB, while CIRTS-7 was co-transfected with the corresponding gRNA construct targeting SMARCA4. (B) Heat map showing knockdown of multiplexed targeting. When both CIRTS-6 and CIRTS-7 are present in cells, co-transfection of either on-target or off-target control gRNA can guide the CIRTS to decrease endogenous transcripts. When both CIRTS have an on-target gRNA for PPIB or SMARCA4 present, both transcripts can be knocked down in the same samples.

FIG. 6. Comparing CIRTS to other DNA and RNA-targeting CRISPR/Cas systems. Schematic size comparison of currently used Cas9, Cas13, and fusion protein systems. Cas9 and Cas13-based delivery systems are substantially larger than engineered CIRTS systems.

FIG. 7A-C. Controls EMSA and Cleavage Assay. (A) EMSA evaluating binding shifts dependent on MBP-CIRTS-1 in the absence of any gRNA. (B) EMSA assaying binding shifts in the presence of only labeled substrate (R1) and on-target gRNA (R3). (C) Full cleavage gel shown in main text FIG. 2C.

FIG. 8A-D. CIRTS linker and gRNA optimization. (A) Luciferase assay with the CIRTS nuclease system using different linkers between the hairpin-binding protein and the effector protein. (B) Luciferase assay with CIRTS-YTHDF2-mediated decay using different linkers between the hairpin-binding protein and the effector protein. (C) Different engineered gRNA for TBP6.7 based on the design shown in FIG. 1C. Two different targeting lengths of 20 and 40 nucleotides were used in combination with different numbers of linking nucleotides (L) between the hairpin and the guiding sequence. The dual luciferase assay was used to assess nuclease-mediated decay. (D) The same engineered gRNAs as in FIG. 8C were used with CIRTS-3 to induce epitranscriptome-induced RNA decay. n=3 biological replicates

FIG. 9A-H. Control luciferase assays and RT-qPCRs. (A) RT-qPCR analysis of RNA levels with the ‘dead’ Pin nuclease domain CIRTS (CIRTS-0). (B) Luciferase assay comparing the nuclease-mediated decay of TBP6.7-Pin nuclease domain without (CIRTS-8) and with (CIRTS-9) the additional ssRNA binding protein ORFS. (C) CIRTS nuclease can be mediate decreases in RNA and therefore protein level in both the nucleus and the cytoplasm (n=6). (D) Comparison of RNA levels when cells were transfected with CIRTS-1 and active Cas13b nuclease. CIRTS-1-Pin mediated RNA cleavage showed substantially less RNA degradation compared to the Cas13b system. (E-H) All engineered CIRTS system the inventors tested in the dual luciferase assay were also subjected to RT-qPCR analysis to assess changes in RNA levels. CIRTS-2, which contain the YTHDF1 effector domain inducing translation activation showed no significant changes in RNA level while all YTHDF2-containing CIRTS show the expected decrease in RNA levels (S3F and S3H: n=2 or 3). n=3 biological replicates unless otherwise noted. Student t-test: *P<0.05, **P<0.01, ***P<0.001.

FIG. 10A-C. Immunoprecipitation, Control qPCR Western Blot, Y2 truncations. (A) Cells were transfected with CIRTS-0-3xFLAG and a gRNA for either PPIB, B4GALTN1, or NT. After crosslinking and FLAG IP, pulled down RNA was quantified using RT-qPCR. Reactions containing on-target gRNA for either transcript showed 3.5 to 5-fold enrichment for these transcripts, indicating guided RNA targeting (n=2 or 3). (B) RT-qPCR analysis of RNA level when cells were transfected with CIRTS-2. As anticipated, no significant changes in RNA level were observed when a YTHDF1-containing protein was used. (C) Different truncations of YTHDF2 were assayed to determine which would be more efficient. The inventors compared luciferase data (left) with qPCR data (right) and concluded to use the Y2(100-200) construct for luciferase analysis and the Y2(1-200) construct for endogenous targeting to enable the best possible quantifications of the tools. n=3 biological replicates unless otherwise noted. Student t-test: *P<0.05, **P<0.01.

FIG. 11A-C. Endogenous targeting with CIRTS. (A) Changes in RNA levels assessed after transfection of CIRTSS-7 alone for PPIB. (B) Similar to FIG. 11A, SMARCA4 levels were assayed when cells were transfected with CIRTSS-7. (C) gRNA screen along SMARCA4 using CIRTS-3 to induce gRNA-dependent RNA decay. The inventors see significant changes in the amount of induce decay dependent on where the transcript is targeted (n=2 or 3). n=3 biological replicates unless otherwise noted. Student t-test: *P<0.05, **P<0.01, ***P<0.001.

FIG. 12A-D. Design of CRISPR/Cas-inspired RNA targeting system (CIRTS) (A) Schematic overview of the design strategy. CIRTS is composed of a ssRNA binding protein, an RNA hairpin binding protein, an effector protein, and a guiding RNA. (B) List of key CIRTS used in this work. (C) Design of the guiding RNA for TBP6.7. The HIV TAR hairpin was fused to a nucleotide linker (L) and a guide sequence. The nucleotide linker was altered during optimization (as described in the supporting information) but L=UUAUU was used for all work thereafter. (D) Design of the guiding RNA for the RNA recognition motif (RRM) of SLBP. The human histone mRNA hairpin was fused to a flexible five nucleotide linker and a guide sequence.

FIG. 13A-B. CIRTS-1 in vitro binding and RNA cleavage assays. (A) Filter binding assay evaluating the binding affinity of MBP-CIRTS-1 with on-target gRNA and non-targeting RNA complex to a labeled RNA substrate. Fitting the data to a quadratic binding equation revealed an apparent KD of 22±7 nM for the on-target:protein complex and an apparent KD around 500 nM for the non-targeting:protein interaction. (B) Cleavage assay run on a 10% denaturing Urea PAGE gel in presence of 0.5 mM MnCl₂. An IR800-labeled RNA substrate is cleaved in a gRNA-dependent manner.

FIG. 14A-G. CIRTS mammalian cell reporter assays. (A) General overview of the dual luciferase assay. A reporter construct that contains both firefly luciferase and Renilla luciferase is used in all assays. The inventors targeted CIRTS to the firefly luciferase transcript, while using Renilla luciferase as an internal control. For all subsequent assays, HEK293T cells were transfected with the reporter vector, a CIRTS vector, and a gRNA vector. (B) Catalytically-inactive CIRTS-0 was used as a control. After 48 h of incubation, the inventors observed no decrease in protein readout. Values shown as mean±SEM with n=3 biological replicates. (C) Comparison of CIRTS-1 with Cas13b nuclease. Cells transfected with either CIRTS-1 or Cas13 and the corresponding gRNA targeting Fluc show reduced protein levels after incubation. Values shown as mean±SEM with n=3 biological replicates. Student t-test: *P<0.05, **P<0.01. (D) HEK293T cells transfected with CIRTS-2 show an increase in protein level after 48 h. Values shown as mean±SEM with n=3 biological replicates. Student t-test: **P<0.01. (E) HEK293T cells transfected with CIRTS-3 show the anticipated decrease in protein level. Values shown as mean±SEM with n=3 biological replicates. Student t-test: *P<0.05. (F) Switching the hairpin-binding protein to SLBP still results in decrease protein levels after 48 h. Values shown as mean±SEM with n=3 biological replicates. Student t-test: *P<0.05. (G) Cells transfected with a fully humanized CIRTS (CIRTS-5 and CIRTS-6) system and an on-target gRNA for firefly luciferase result again in decreased protein levels. Values shown as mean±SEM with n=3 biological replicates. Student t-test: *P<0.05.

FIG. 15A-B. CIRTS for RNA editing. (A) Schematic overview of the RNA editing reporter assay used. A single G-to-A mutation was introduced in the coding sequence of firefly luciferase resulting in a W417X (X=STOP) codon switch and no measurable firefly luciferase signal (FIG. 22J). (B) Delivery of CIRTS-7 (hADAR2 wt) and CIRTS-8 (hADAR E488Q) with an on-target gRNA shows significant RNA editing that results in measurable firefly luciferase signal. Both the background and the editing efficiency of CIRTS-8, the hyperactive hADAR2 mutant, are found to be higher compared to wildtype. Values shown as mean±SEM with n=3 biological replicates. Student t-test: ***P<0.001.

FIG. 16A-C. Targeting endogenous transcripts with CIRTS. (A) Nuclease-mediated knockdown of five endogenous transcripts upon transfection of cells with CIRTS-1 as assayed using qPCR. CIRTS-1 can be used to target endogenous transcripts of interest by co-transfecting a gRNA with the corresponding on-target guiding sequence. Values shown as mean±SEM with n=3 biological replicates. Student t-test: *P<0.05, **P<0.01, ***P<0.001. (B) qPCR analysis of YTHDF2-mediated knockdown of endogenous transcripts with CIRTS-3. Cells transfected with CIRTS-3 show gRNA-dependent decreases in RNA level for all five transcripts tested. Values shown as mean±SEM with n=3 biological replicates. Student t-test: *P<0.05. (C) Analysis of protein levels after transfection with CIRTS-2 or CIRTS-3 by Western blot. CIRTS-2 induces an increase in protein levels, whereas CIRTS-3 shows the expected decrease in protein levels, both in a gRNA-dependent manner.

FIG. 17. Targeting accessibility determines knockdown efficiency of CIRTS. gRNA screen along SMARCA4 using CIRTS-3 to induce gRNA-dependent RNA decay. The inventors observe significant changes in the amount of induced decay dependent on where the transcript is targeted (n=2 or 3).

FIG. 18A-D. Multidimensional targeting with CIRTS. (A) Schematic of delivery of CIRTS-6 and three gRNAs. (B) CIRTS-6 can be delivered with three distinct gRNAs for PPIB, SMARCA4, and NRAS and simultaneously cause knockdown of all three transcripts. n=5 biological replicates. Student t-test: **P<0.05, ***P<0.001. (C) Schematic of simultaneous CIRTS delivery with different effector proteins. (D) Changes in luciferase protein levels and PPIB transcript levels when cells were transfected with both CIRTS-9 (YTHDF1) and CIRTS-10 (YTHDF2) and gRNAs for Fluc and PPIB respectively. Both orthogonal CIRTS retain their individual functions and act simultaneously in cells. n=5 biological replicates. Student t-test: *P<0.1, **P<0.05.

FIG. 19A-C. AAV Delivery of CIRTS. (A) Transfer plasmid for AAV delivery containing both the CIRTS-6 (YTHDF2) as well as the gRNA component of the system. The total insert size between the two inverted terminal repeats (ITR) was 2.7 kb. (B) AAV-packaged CIRTS-6 and a gRNA targeting luciferase was delivered to HEK293T cells to knockdown firefly luciferase in the dual luciferase reporter assay. (C) AAV-packaged CIRTS-6 and a gRNA targeting SMARCA4 was delivered to HEK293T cells to knockdown the endogenous gene, which revealed efficiency comparable to that achieved by transient transfection. Values shown as mean±SEM with n=3 biological replicates. Student t-test: *P<0.05.

FIG. 20. Comparing CIRTS to other DNA and RNA-targeting CRISPR/Cas systems. Schematic size comparison of commonly used Cas9, Cas12, Cas13, and fusion protein systems.

FIG. 21. CIRTS List continued from FIG. 12B. Reference list of all remaining CIRTS used in this work.

FIG. 22A-J. Control luciferase assays and RT-qPCRs. (A) Luciferase assay comparing the nuclease-mediated decay of TBP6.7-Pin nuclease domain without (CIRTS-11) and with (CIRTS-12) the additional ssRNA binding protein ORFS. (B) CIRTS nuclease can mediate decreases in RNA and therefore protein level in both the nucleus and the cytoplasm (n=6). (C) RT-qPCR analysis of RNA levels with the ‘dead’ Pin nuclease domain CIRTS (CIRTS-0). (D) Comparison of RNA levels when cells were transfected with CIRTS-1 and active Cas13b nuclease. CIRTS-1-Pin mediated RNA cleavage showed substantially less RNA degradation compared to the Cas13b system. (E-H) All engineered CIRTS system tested in the dual luciferase assay were also subjected to RT-qPCR analysis to assess changes in RNA levels. CIRTS-2, which contain the YTHDF1 effector domain inducing translation activation showed no significant changes in RNA level while all YTHDF2-containing CIRTS show the expected decrease in RNA levels. (I) Engineered CIRTS-18 containing the PP7 dimer as the hairpin binding protein. Knockdown of PPIB after transfection with CIRTS-18 as measured by qPCR. (J) Comparison of reporter only and reporter with CIRTS-7 (hADAR wt) with non-targeting or targeting gRNA (S3F and S3H: n=2 or 3). n=3 biological replicates unless otherwise noted. Student t-test: *P<0.05, **P<0.01, ***P<0.001.

FIG. 23A-D. CIRTS linker and gRNA optimization. (A) Luciferase assay with the CIRTS nuclease system using different linkers between the hairpin-binding protein and the effector protein. Previously published L8=SGSETPGTSESATPES (SEQ ID NO:133) (Guilinger et al., 2014), 10 nm helical linker=EEEEKKKQQEEEAERLRRIQEEMEKERKRREEDEKRRRKEEEERRMKLEMEAKRKQ EEEERKKREDDEKRKKK (SEQ ID NO:134). (B) Luciferase assay with CIRTS-YTHDF2-mediated decay using different linkers between the hairpin-binding protein and the effector protein. (C) Different engineered gRNA for TBP6.7 based on the design shown in FIG. 1C. Two different targeting lengths of 20 and 40 nucleotides were used in combination with different numbers of linking nucleotides (L) between the hairpin and the guiding sequence. The dual luciferase assay was used to assess nuclease-mediated decay. NT=non-targeting, Fluc gRNA containing different linker nuclease (FIG. 1), L2=UU, L3=UUU, L5=UUAUU. (D) The same engineered gRNAs as in Figure S3C were used with CIRTS-3 to induce epitranscriptome-induced RNA decay. NT=non-targeting, Fluc gRNA containing different linker nuclease (FIG. 1), L2=UU, L3=UUU, L5=UUAUU. n=3 biological replicates.

FIG. 24A-D. Control qPCR, Western Blot, YTHDF2 truncations. (A) CIRTS-1 can be delivered to RNA species other than mRNA. As a proof-of-principle, the inventors transfected cells with CIRTS-1 (Pin nuclease) and two different gRNAs for the lncRNA MALAT1 and assessed RNA levels by RT-qPCR. (B) RT-qPCR analysis of RNA level when cells were transfected with CIRTS-2. As anticipated, no significant changes in RNA level were observed when a YTHDF1-containing protein was used. (C) Quantification of protein levels as measured by Western blot when cells were transfected with CIRTS-2 or CIRTS-3 and targeted to PPIB (n=3). (D) Different truncations of YTHDF2 were assayed to determine which would be more efficient. The inventors compared luciferase data (left) with qPCR data (right) and concluded to use the Y2(100-200) construct for luciferase analysis and the Y2(1-200) construct for endogenous targeting to enable the best possible quantifications of the tools. n=3 biological replicates unless otherwise noted. Student t-test: *P<0.05, **P<0.01.

FIG. 25A-H. Targeting Specificity of CIRTS. (A) Schematic of the KRAS4b-luciferase mismatch reporter assay. The inventors chose four KRAS4b variants that have an increasing number of mismatches to the designed 20 nt length gRNA and fused it N-terminal to the dual luciferase reporter. (B) CIRTS-mediated knockdown of KRAS4b-Fluc with different numbers of mismatches between the gRNA and target RNA as described in Figure S5A. CIRTS was found to be most sensitive to mismatches in the middle of its guiding sequence. (C) Cas13b-mediated knockdown in the same KRAS4b-Fluc reporter assay as described above. Cas13b shows a higher knockdown efficiency but is also less sensitive to mismatches introduced. Similar to CIRTS, Cas13b is knockdown is most affected by mismatches at the center of the guiding target duplex region. (D) Knockdown efficiency of CIRTS on the KRAS4b-luciferase mismatch reporter when using a 40 nt gRNA length. A longer guiding sequence in the gRNA can rescue some of the loss in knockdown efficiency. (E-F) Mean expression levels of the transcriptome in log₂(transcript per million (TPM)+1) when CIRTS Pin nuclease (E) or CIRTS YTHDF2 (F) are deployed to SMARCA4 in cells (n=3). (G) Knockdown levels of SMARCA4 as determined by RNA sequencing. (H) Cells were transfected with CIRTS-0-3xFLAG and a gRNA for either PPIB, B4GALNT1, or NT.

After crosslinking and FLAG IP, pulled down RNA was quantified using RT-qPCR. Reactions containing on-target gRNA for either transcript showed 3.5 to 5-fold enrichment for these transcripts, indicating guided RNA targeting (n=2 or 3).

FIG. 26A-C. Endogenous targeting with CIRTS. (A) Changes in RNA levels as assessed by RT-qPCR after transfection of CIRTSS-7 alone for PPIB. (B) Similar to Figure S6A, SMARCA4 levels were assayed when cells were transfected with CIRTSS-7. (C) Comparison of knockdown levels of PPIB after delivery of active Cas13b nuclease or an engineered dCas13b-YTHDF2(1-200) construct to PPIB (n=2 or 3). n=3 biological replicates unless otherwise noted. Student t-test: *P<0.01, **P<0.05, ***P<0.01, ****P<0.001.

FIG. 27A-C. Multiplexed targeting with CIRTS. (A) Schematic of vectors used for multiplexed targeting. Cells were transfected with an expression vector for CIRTS-6, and an expression vector for CIRTS-10, an expression vector for a CIRTS-6 gRNA construct targeting PPIB or a non-targeting control, and an expression vector for a CIRTS-9 gRNA targeting SMARCA4 or a non-targeting control. (B) Heat map showing knockdown of multiplexed targeting described in (A). When both CIRTS have an on-target gRNA for PPIB or SMARCA4 present, both transcripts can be knocked down in the same samples. Values shown as mean expression level of each target transcript relative to GAPDH, with n=8 biological replicates. (C) Computational prediction of immunogenicity. The inventors first predicted 9-mer peptides that are MHC I binders using the IEDB database and subjected the top one percentile of binders to immunogenicity predictions using the IEDB immunogenicity predictor.

FIG. 28. gRNA screen with ADAR. Testing whether guide RNA designs that feature multiple hairpins increase the potency of CIRTS. Left panel shows gRNA designs feature either the origins design, or guides with one TAR hairpin on either end of the guide, or two hairpins on either end. The additional hairpin guides increase the potency of CIRTS in an

ADAR activity assay in cells. Right panel is testing whether the second hairpin needs to be a TAR hairpin, or if just a “stabilizing hairpin” can function—meaning a hairpin that does not directly interact with the CIRTS protein but slows degradation. As seen in the data, the second hairpin increase potency compared to one hairpin gRNA design.

FIG. 29A-B. (A) C-to-U Editor: The data in A demonstrates that a CIRTS based on a C-to-U base editor is also functional using a mammalian cell reporter assay. (B) ssRNA binding proteins. The data in B demonstrates that other ssRNA binding proteins can function as a RNA binding proteins in the systems and methods of the disclosure.

FIG. 30A-C. (A): RNA regulatory domain-containing proteins that may activate translation, arraying gRNAs at various locales on a reporter RNA and measuring translational activation of each. (B-C). RNA regulatory domain-containing proteins that potentially degrade or destabilize an RNA, arraying gRNAs at various locales on a reporter RNA and measuring RNA degradation of each.

FIG. 31A-B. (A) Embodiments demonstrating the different orientations of the elements of the systems of the disclosure. (B) Data using CNOT7 in different orientations (as shown in A) and on two RNA targets: a luciferase reporter (left) and an endogenous RNA (right). Several different orientations of the proteins still function, indicating the proteins can be engineered in differed orders depending on the needs of the effector.

FIG. 32A-D. A CIRTS biosensor for inducible RNA targeting. (A) Schematic overview of the abscisic acid (ABA) CIRTS biosensor design. The gRNA-mediated targeting component of CIRTS is fused to one of the ABA heterodimerization domains (ABI) while the effector component of CIRTS is fused to its binding partner (PYL). Upon addition of the small molecule ABA, the two CIRTS components dimerize and bring the effector in proximity of the targeted transcript. (B) ABA-inducible RNA degradation of an red-fluorescent protein (RFP) reporter transcript 48 h after transfection can be mediated by the Pin nuclease domain or YTHDF2. (C) Translation activation of RFP by ABA-induced CIRTS-YTHDF1 48 h after transfection. (D) Delivery of CIRTS-hADAR with on-target gRNA in the presence of ABA to cells transfected with a mutation-deactivated luciferase reporter (FlucW417X for A-to-I editing or the GlucC82R for C-to-U editing) induces ABA-dependent RNA editing.

### DESCRIPTION OF ILLUSTRATIVE EMBODIMENTS

Epitranscriptomic regulation controls information flow through the central dogma and provides unique opportunities for manipulating cell state at the RNA level. However, both fundamental mechanistic studies and potential translational applications are impeded by a lack of effective methods to target specific RNAs with effector proteins. Here, the inventors present the design and validation of a CRISPR/Cas-inspired RNA targeting system (CIRTS), a new protein engineering strategy for constructing programmable RNA regulatory systems. The inventors show that CIRTS is a simple and generalizable approach to deliver a range of effector proteins, including nucleases, degradation machinery, and translational activators, to target transcripts. CIRTS are not only smaller than naturally-occurring CRISPR/Cas programmable RNA binding systems, but can be built entirely from human protein parts. The small size and human-derived nature of CIRTS provides a less perturbative method for fundamental RNA regulatory studies as well as a potential strategy to avoid immune issues when applied to epitranscriptome-modulating therapies.

### I. RNA REGULATORY DOMAIN

It is contemplated that any RNA regulatory domain may be used in the methods and systems of the current disclosure. For example, a RNA regulatory domain with one or more of the following activities may be used: methylation, 5′-3′ guanylylation, phosphoribosylation, deamination, carbamoylation, isopentenylation, agmatinylation, acetylation, lysylation, O/S exchange, galactosylation, glutamylation, mannosylation, hydrogenation, pseudouridine formation, carboxymethylaminomethylation, aminomethylation, decarboxymethylation, dehydrogenation, carboxymethylation, hydroxylation, methylthiolation, 3-amino-3-carboxypropylation, demethylation, 5′-5′ guanylylation, and dephosphorylation.

Exemplary RNA regulatory domains include domains from the following proteins of Table 1 (or functional fragments thereof):

TABLE 1

| Acronym | Full Name | Organism | Activity |
|---|---|---|---|
| BCDIN3D | BCDIN3 domain containing | Homo sapiens | methylation |
| Nop1 | rRNA 2′-O-methyltransferase | Saccharomyces | methylation |
| | fibrillarin | cerevisiae | |
| Nsr | rRNA (adenosine-2′-O-)- | Streptomyces | methylation |
| | methyltransferase | actuosus | |
| Trm13 | tRNA-2′-0-methyltransferase | Saccharomyces | methylation |
| | TRM13 | cerevisiae | |
| Tsr | rRNA (adenosine-2′-O-)- | Streptomyces | methylation |
| | methyltransferase | cyaneus | |
| KamB | rRNA adenine N-1- | Streptomyces sp. | methylation |
| | methyltransferase | DSM 40477 | |
| NpmA | rRNA adenine N-1- | Escherichia coli | methylation |
| | methyltransferase | | |
| Rrp8 | Ribosomal RNA-processing | Saccharomyces | methylation |
| | protein 8 | cerevisiae | |
| TRMT10C | tRNA methyltransferase 10 C, | Homo sapiens | methylation |
| | mitochondrial | | |
| Trm61 | tRNA (adenine(58)-N(1))- | Saccharomyces | methylation |
| | methyltransferase catalytic | cerevisiae | |
| | subunit TRM61 | | |
| TrmI | tRNA (adenine(57)- | Pyrococcus abyssi | methylation |
| | N(1)/adenine(58)-N(1))- | | |
| | methyltransferase TrmI | | |
| TrmI | tRNA (adenine(58)-N(1))- | Thermus | methylation |
| | methyltransferase TrmI | thermophilus | |
| TrmI | tRNA (adenine(58)-N(1))- | Mycobacterium | methylation |
| | methyltransferase TrmI | tuberculosis | |
| TrmK | tRNA (adenine(22)-N(1))- | Bacillus subtilis | methylation |
| | methyltransferase | | |
| Trmt61A | tRNA (adenine(58)-N(1))- | Homo sapiens | methylation |
| | methyltransferase catalytic | | |
| | subunit TRMT61A | | |
| Trmt61B | tRNA (adenine(58)-N(1))- | Homo sapiens | methylation |
| | methyltransferase, mitochondrial | | |
| METTL14 | | Homo sapiens | methylation |
| METTL3 | N6-adenosine-methyltransferase 70 | Homo sapiens | methylation |
| | kDa subunit | | |
| RsmA | Dimethyladenosine transferase | Saccharomyces | methylation |
| | | cerevisiae | |
| ErmAM | rRNA adenine N-6- | Streptococcus | methylation |
| | methyltransferase | pneumoniae | |
| Cfr | Ribosomal RNA large subunit | Staphylococcus | methylation |
| | methyltransferase Cfr | sciuri | |
| Nop1 | rRNA 2′-O-methyltransferase | Saccharomyces | methylation |
| | fibrillarin | cerevisiae | |
| Dnmt2 | tRNA (cytosine(38)-C(5))- | Homo sapiens | methylation |
| | methyltransferase | | |
| Trm4 | tRNA (cytosine(34)-C(5))- | Homo sapiens | methylation |
| | methyltransferase | | |
| MRM1 | rRNA methyltransferase 1, | Homo sapiens | methylation |
| | mitochondrial | | |
| RNMTL1 | RNA methyltransferase-like | Homo sapiens | methylation |
| | protein 1 | | |
| TRM5 | tRNA (guanine(37)-N1)- | Homo sapiens | methylation |
| | methyltransferase | | |
| TRMT10A | tRNA methyltransferase 10 | Homo sapiens | methylation |
| | homolog A | | |
| TRMT10B | tRNA methyltransferase 10 | Homo sapiens | methylation |
| | homolog B | | |
| TRMT10C | tRNA methyltransferase 10 | Homo sapiens | methylation |
| | C, mitochondrial | | |
| TRMB | tRNA (guanine-N(7)-)- | Homo sapiens | methylation |
| | methyltransferase | | |
| Hcm1 | RNA cap guanine-N7 | Homo sapiens | methylation |
| | methyltransferase | | |
| MRM2 | rRNA methyltransferase 2, | Homo sapiens | methylation |
| | mitochondrial | | |
| ALKBH8 | Alkylated DNA repair protein alkB | Homo sapiens | methylation |
| | homolog 8 | | |
| BCDIN3D | BCDIN3 domain containing | Homo sapiens | methylation |
| Tgs1 | trimethylguanosinesynthase 1 | Homo sapiens | methylation |
| Trm1 | tRNA (guanine(26)-N(2))- | Homo sapiens | methylation |
| | dimethyltransferase | | |
| Thg1 | tRNA(His) guanylyltransferase | Saccharomyces | 5′-3′ guanylylation |
| | | cerevisiae | |
| Rit1 | tRNA A64-2′-O-ribosylphosphate | Saccharomyces | phosphoribosylation |
| | transferase | cerevisiae | |
| Tad1 | tRNA-specific adenosine | Saccharomyces | deamination |
| | deaminase 1 | cerevisiae | |
| Tad1 | tRNA-specific adenosine | Homo sapiens | deamination |
| | deaminase 1 | | |
| Tad2 | tRNA-specific adenosine deaminase | Saccharomyces | deamination |
| | subunit TAD2 | cerevisiae | |
| Tad3 | tRNA-specific adenosine deaminase | Saccharomyces | deamination |
| | subunit TAD3 | cerevisiae | |
| TadA | tRNA-specific adenosine deaminase | Escherichia coli | deamination |
| TadA | tRNA-specific adenosine deaminase, | Arabidopsis | deamination |
| | chloroplastic | thaliana | |
| CDAT8 | tRNA-specific cytidine deaminase | Methanopyrus | deamination |
| | | kandleri | |
| MiaA | tRNA (adenosine(37)-N6)- | Escherichia coli | isopentenylation |
| | dimethylallyltransferase | | |
| Mod5 | tRNA dimethylallyltransferase, | Saccharomyces | isopentenylation |
| | mitochondrial | cerevisiae | |
| Mod5 | tRNA dimethylallyltransferase, | Homo sapiens | isopentenylation |
| | mitochondrial | | |
| Sua5 | tRNA threonylcarbamoyladenosine | Saccharomyces | carbamoylation |
| | biosynthesis protein SUA5 | cerevisiae | |
| TsaD | tRNA N6- | Escherichia coli | carbamoylation |
| | threonylcarbamoyladenosine(37) | | |
| | synthesis protein | | |
| TiaS | tRNA(Ile2) 2-agmatinylcytidine | Archaeoglobus | agmatinylation |
| | synthase TiaS | fulgidus | |
| NAT10 | N-acetyltransferase 10 | Homo sapiens | acetylation |
| TilS | tRNA lysidine(34) synthetase | Escherichia coli | lysylation |
| TtcA | tRNA 2-thiocytidine(32) synthetase | Escherichia coli | O/S exchange |
| TrmU | Mitochondrial tRNA-specific 2- | Homo sapiens | O/S exchange |
| | thiouridylase 1 | | |
| Man/Gal-Q- | tRNA-queuine glycosyltransferase | various eukaryotes | galactosylation |
| transferase | | | |
| GluQRS | tRNA glutamyl-Q(34) synthetase | Escherichia coli | glutamylation |
| Man/Gal-Q- | tRNA-queuine glycosyltransferase | various eukaryotes | mannosylation |
| transferase | | | |
| Dus1 | tRNA-dihydrouridine synthase 1 | Saccharomyces | hydrogenation |
| | | cerevisiae | |
| DusA | tRNA dihydrouridine synthase A | Escherichia coli | hydrogenation |
| Pus1 | tRNA pseudouridine synthase 1, | Homo sapiens | pseudouridine |
| | mitochondrial | | formation |
| Pus1 | tRNA pseudouridine synthase 1 | Saccharomyces | pseudouridine |
| | | cerevisiae | formation |
| RluA | tRNA pseudouridine(32) synthase/ | Escherichia coli | pseudouridine |
| | Ribosomal large subunit | | formation |
| | pseudouridine synthase A | | |
| MnmE | tRNA uridine(34) 5- | Escherichia coli | carboxymethylamino- |
| | carboxymethylaminomethyl | | methylation |
| | synthesis GTPase | | |
| MnmE | tRNA uridine(34) 5- | Escherichia coli | aminomethylation |
| | carboxymethylaminomethyl | | |
| | synthesis GTPase | | |
| MnmCD | tRNA (5-methylaminomethy1-2- | Escherichia coli | decarboxymethylation |
| | thiouridylate)-methyltransferase/ | | |
| | FAD-dependent cmnm(5)s(2)U34 | | |
| | oxidoreductase | | |
| FTO | Alpha-ketoglutarate-dependent | Homo sapiens | dehydrogenation |
| | dioxygenase FTO | | |
| CmoB | tRNA 5-methoxyuridine(34) | Escherichia coli | carboxymethylation |
| | synthase | | |
| MiaE | tRNA 2-methylthio-N6-isopentenyl | Salmonella | hydroxylation |
| | adenosine(37) hydroxylase | typhimurium | |
| FTO | Alpha-ketoglutarate-dependent | Homo sapiens | hydroxylation |
| | dioxygenase FTO | | |
| ALKBH8 | Alkylated DNA repair protein alkB | Homo sapiens | hydroxylation |
| | homolog 8 | | |
| MiaB | tRNA (N6-isopentenyl | Escherichia coli | methylthiolation |
| | adenosine(37)-C2)- | | |
| | methylthiotransferase | | |
| TYW2 | tRNA wybutosine-synthesizing | Saccharomyces | 3-amino-3- |
| | protein 2 | cerevisiae | carboxypropylation |
| ALKBH5 | | Homo sapiens | demethylation |
| FTO | Alpha-ketoglutarate-dependent | Homo sapiens | demethylation |
| | dioxygenase FTO | | |
| HCAP 1 | RNA-capping enzyme | Homo sapiens | 5′-5′ guanylylation |
| HCAP 1 | RNA-capping enzyme | Homo sapiens | dephosphorylation |

Further RNA regulatory domains include functional domains from the following human proteins of Table 2:

TABLE 2

| Gene Symbol | Synonyms | Gene Description |
|---|---|---|
| RBM5 | LUCA15; H37 | RNA binding motif protein 5 [Source:HGNC |
| | | Symbol;Acc:9902] |
| RBM6 | DEF-3; 3G2; NY- | RNA binding motif protein 6 [Source:HGNC |
| | LU-12; g16; DEF3 | Symbol;Acc:9903] |
| | | Putative RNA exonuclease NEF-sp (EC 3.1.—.—) |
| | | [Source:UniProtKB/Swiss-Prot;Acc:Q96IC2] |
| YBX2 | MSY2; CSDA3 | Y box binding protein 2 [Source:HGNC |
| | | Symbol;Acc:17948] |
| CSDE1 | D15155E; UNR | cold shock domain containing E1, RNA-binding |
| | | [Source:HGNC Symbol;Acc:29905] |
| PTBP1 | HNRPI; HNRNP-I; | polypyrimidine tract binding protein 1 [Source:HGNC |
| | PTB2; PTB3; | Symbol;Acc:9583] |
| | PTB-1; PTB4; | |
| | pPTB | |
| ZC3H3 | KIAA0150 | zinc finger CCCH-type containing 3 [Source:HGNC |
| | | Symbol;Acc:28972] |
| MATR3 | KIAA0723; | matrin 3 [Source:HGNC Symbol;Acc:6912] |
| | MGC9105 | |
| SAMD4A | KIAA1053; | sterile alpha motif domain containing 4A [Source: |
| | DKFZP434H0350; | HGNC Symbol;Acc:23023] |
| | Smaug; SMG; | |
| | SMGA; hSmaug1 | |
| YTHDC2 | FLJ2194; | YTH domain containing 2 [Source:HGNC |
| | FLJ10053; | Symbol;Acc:24721] |
| | DKFZp564A186 | |
| CUGBP2 | | CUG triplet repeat, RNA binding protein 2 |
| | | [Source:HGNC Symbol;Acc:2550] |
| PUM2 | PUMH2; | pumilio homolog 2 (Drosophila) [Source:HGNC |
| | KIAA0235 | Symbol;Acc:14958] |
| RC3H2 | FLJ20301; | ring finger and CCCH-type zinc finger domains 2 |
| | FLJ20713; | [Source:HGNC Symbol;Acc:21461] |
| | RNF164 | |
| ZC3H11A | KIAA0663 | zinc finger CCCH-type containing 11A [Source:HGNC |
| | | Symbol;Acc:29093] |
| PARP12 | FLJ22693; PARP- | poly (ADP-ribose) polymerase family, member 12 |
| | 12; ZC3H1 | [Source:HGNC Symbol;Acc:21919] |
| CSDA | dbpA; ZONAB; | cold shock domain protein A pseudogene 1 |
| | CSDA1 | [Source:HGNC Symbol;Acc:2429] |
| SFRS8 | SWAP | splicing factor, arginine/serine-rich 8 (suppressor-of- |
| | | white-apricot homolog, Drosophila) [Source:HGNC |
| | | Symbol;Acc:10790] |
| EIF4B | | eukaryotic translation initiation factor 4B |
| | | [Source:HGNC Symbol;Acc:3285] |
| U2AF2 | U2AF65 | U2 small nuclear RNA auxiliary factor 2 [Source: |
| | | HGNC Symbol;Acc:23156] |
| SFRS14 | KIAA0365 | splicing factor, arginine/serine-rich 14 [Source:HGNC |
| | | Symbol;Acc:18641] |
| SPEN | KIAA0929; MINT; | spen homolog, transcriptional regulator (Drosophila) |
| | SHARP; RBM15C | [Source:HGNC Symbol;Acc:17575] |
| ZC3H15 | LEREPO4 | zinc finger CCCH-type containing 15 [Source:HGNC |
| | | Symbol;Acc:29528] |
| YBX1 | YB-1; YB1; | Y box binding protein 1 [Source:HGNC |
| | DBPB; NSEP-1; | Symbol;Acc:8014] |
| | MDR-NF1; BP-8; | |
| | CSDB; CSDA2 | |
| ELAVL1 | HuR; Hua; MelG | ELAV (embryonic lethal, abnormal vision, Drosophila)- |
| | | like 1 (Hu antigen R) [Source:HGNC Symbol;Acc:3312] |
| THUMPD1 | FLJ20274 | THUMP domain containing 1 [Source:HGNC |
| | | SymbokAcc:23807] |
| DHX8 | HRH1; PRP22; | DEAH (Asp-Glu-Ala-His) box polypeptide 8 |
| | PRPF22 | [Source:HGNC Symbol;Acc:2749] |
| SRBD1 | FLJ10379 | S1 RNA binding domain 1 [Source:HGNC |
| | | Symbol;Acc:25521] |
| PABPC1 | PABP1; PABPL1 | poly(A) binding protein, cytoplasmic 1 [Source:HGNC |
| | | Symbol;Acc:8554] |
| DAZAP1 | MGC19907 | DAZ associated protein 1 [Source:HGNC |
| | | Symbol;Acc:2683] |
| IGF2BP2 | IMP-2 | insulin-like growth factor 2 mRNA binding protein 2 |
| | | [Source:HGNC Symbol;Acc:28867] |
| ZNF638 | NP220; | zinc finger protein 638 [Source:HGNC |
| | MGC26130; | Symbol;Acc:17894] |
| | Zfp638 | |
| SART3 | KIAA0156; RP11- | squamous cell carcinoma antigen recognized by T cells 3 |
| | 13G14 | [Source:HGNC Symbol;Acc:16860] |
| MKRN2 | RNF62; HSPC070 | makorin ring finger protein 2 [Source:HGNC |
| | | Symbol;Acc:7113] |
| RBM7 | | RNA binding motif protein 7 [Source:HGNC |
| | | Symbol;Acc:9904] |
| RBMS2 | SCR3 | RNA binding motif, single stranded interacting protein 2 |
| | | [Source:HGNC Symbol;Acc:9909] |
| MBNL3 | CHCR; FLJ11316; | muscleblind-like 3 (Drosophila) [Source:HGNC |
| | MBLX39; MBXL | Symbol;Acc:20564] |
| SNRPA | U1A; U1-A; Mud1 | small nuclear ribonucleoprotein polypeptide A |
| | | [Source:HGNC Symbol;Acc:11151] |
| SYNJ2 | INPP5H | synaptojanin 2 [Source:HGNC Symbol;Acc:11504] |
| A2BP1 | | Fox-1 homolog A (Ataxin-2-binding protein |
| | | 1)(Hexaribonucleotide-binding protein 1) |
| | | [Source:UniProtKB/Swiss-Prot;Acc:Q9NWB1] |
| | | Trinucleotide repeat-containing gene 6C protein |
| | | [Source:UniProtKB/Swiss-Prot;Acc:Q9HCJO] |
| DDX43 | HAGE; | DEAD (Asp-Glu-Ala-Asp) box polypeptide 43 |
| | DKFZp434H2114. | [Source:HGNC Symbol;Acc:18677] |
| | CT13 | |
| KIAA0020 | XTP5; PEN; PUF6 | KIAA0020 [Source:HGNC Symbol;Acc:29676] |
| CNOT4 | CLONE243; | CCR4-NOT transcription complex, subunit 4 |
| | NOT4H | [Source:HGNC Symbol;Acc:7880] |
| YTHDC1 | YT521; | YTH domain containing 1 [Source:HGNC |
| | KIAA1966; | Symbol;Acc:30626] |
| | YT521-B | |
| PPIE | CyP-33; | peptidylprolyl isomerase E (cyclophilin E) |
| | MGC3736; | [Source:HGNC Symbol;Acc:9258] |
| | MGC111222 | |
| CHERP | ERPROT213-21; | calcium homeostasis endoplasmic reticulum protein |
| | DAN16 | [Source:HGNC Symbol;Acc:16930] |
| RBM22 | FLJ10290; | RNA binding motif protein 22 [Source:HGNC |
| | ZC3H16; fSAP47; | Symbol;Acc:25503] |
| | Cwc2 | |
| KHSRP | KSRP; FBP2; | KH-type splicing regulatory protein [Source:HGNC |
| | FUBP2 | Symbol;Acc:6316] |
| FUS | TLS; FUS1; | fused in sarcoma [Source:HGNC Symbol;Acc:4010] |
| | hnRNP-P2 | |
| RBM41 | FLJ11016 | RNA binding motif protein 41 [Source:HGNC |
| | | Symbol;Acc:25617] |
| PCBP4 | MCG10; LIP4 | poly(rC) binding protein 4 [Source:HGNC |
| | | Symbol;Acc:8652] |
| PABPC4 | iPABP; APP-1 | poly(A) binding protein, cytoplasmic 4 (inducible form) |
| | | [Source:HGNC Symbol;Acc:8557] |
| TNRC6A | CAGH26; | trinucleotide repeat containing 6A [Source:HGNC |
| | KIAA1460; | Symbol;Acc:11969] |
| | GW182 | |
| RBM27 | KIAA1311; | RNA binding motif protein 27 [Source:HGNC |
| | ARRS1; Psc1 | Symbol;Acc:29243] |
| HNRNPC | hnRNPC | heterogeneous nuclear ribonucleoprotein C (C1/C2) |
| | | [Source:HGNC Symbol;Acc:5035] |
| DAZL | DAZH; SPGYLA; | deleted in azoospermia-like [Source:HGNC |
| | MGC26406; | Symbol;Acc:2685] |
| | DAZL1 | |
| HNRNPH3 | 2H9 | heterogeneous nuclear ribonucleoprotein H3 (2H9) |
| | | [Source:HGNC Symbol;Acc:5043] |
| SETD1A | KIAA0339; Set1; | SET domain containing 1A [Source:HGNC |
| | KMT2F | Symbol;Acc:29010] |
| CIRBP | CIRP | cold inducible RNA binding protein [Source:HGNC |
| | | Symbol;Acc:1982] |
| HNRNPM | HTGR1; | heterogeneous nuclear ribonucleoprotein M |
| | HNRNPM4; | [Source:HGNC Symbol;Acc:5046] |
| | HNRPM4 | |
| TRMT2A | HTF9C | TRM2 tRNA methyltransferase 2 homolog A (S. |
| | | cerevisiae) [Source:HGNC Symbol;Acc:24974] |
| SF3A1 | SF3a120; SAP114; | splicing factor 3a, subunit 1, 120kDa [Source:HGNC |
| | PRPF21; Prp21 | Symbol;Acc:10765] |
| SNRPD3 | SMD3; Sm-D3 | small nuclear ribonucleoprotein D3 polypeptide 18kDa |
| | | [Source:HGNC Symbol;Acc:11160] |
| POLDIP3 | PDIP46; | polymerase (DNA-directed), delta interacting protein 3 |
| | KIAA1649 | [Source:HGNC Symbol;Acc:23782] |
| ZMAT5 | | zinc finger, matrin type 5 [Source:HGNC |
| | | Symbol;Acc:28046] |
| RBM9 | HNRBP2; FOX-2 | RNA binding motif protein 9 [Source:HGNC |
| | | Symbol;Acc:9906] |
| ZC3H7B | RoXaN; | zinc finger CCCH-type containing 7B [Source:HGNC |
| | FLJ13787; | Symbol;Acc:30869] |
| | DKFZp434K0920; | |
| | KIAA1031 | |
| RBM23 | FLJ10482 | RNA binding motif protein 23 [Source:HGNC |
| | | Symbol;Acc:20155] |
| SFRS5 | SRP40; HRS | splicing factor, arginine/serine-rich 5 [Source:HGNC |
| | | Symbol;Acc:10787] |
| ZC3H14 | FLJ11806; UKp68; | zinc finger CCCH-type containing 14 [Source:HGNC |
| | NY-REN-37 | Symbol;Acc:20509] |
| ACIN1 | KIAA0670; | apoptotic chromatin condensation inducer 1 |
| | fSAP152 | [Source:HGNC Symbol;Acc:17066] |
| PABPN1 | PAB2 | poly(A) binding protein, nuclear 1 [Source:HGNC |
| | | Symbol;Acc:8565] |
| PABPC1L | dJ1069P2.3; | poly(A) binding protein, cytoplasmic 1-like |
| | PABPC1L1; ePAB | [Source:HGNC Symbol;Acc:15797] |
| BRUNOL4 | | bruno-like 4, RNA binding protein (Drosophila) |
| | | [Source:HGNC Symbol;Acc:14015] |
| CSTF2 | | cleavage stimulation factor, 3′pre-RNA, subunit 2, |
| | | 64kDa [Source:HGNC Symbol;Acc:2484] |
| ZC3H12B | MCPIP2 | zinc finger CCCH-type containing 12B [Source:HGNC |
| | | Symbol;Acc:17407] |
| FMR1 | FMRP; FRAXA; | fragile X mental retardation 1 [Source:HGNC |
| | MGC87458 | Symbol;Acc:3775] |
| HTATSF1 | TAT-SF1 | HIV-1 Tat specific factor 1 [Source:HGNC |
| | | Symbol;Acc:5276] |
| RBM3 | IS1-RNPL | RNA binding motif (RNP1, RRM) protein 3 |
| | | [Source:HGNC Symbol;Acc:9900] |
| ESRP2 | FLJ21918 | epithelial splicing regulatory protein 2 [Source:HGNC |
| | | Symbol;Acc:26152] |
| MTHFSD | FLJ12998 | methenyltetrahydrofolate synthetase domain containing |
| | | [Source:HGNC Symbol;Acc:25778] |
| DNAJC17 | FLJ10634 | DnaJ (Hsp40) homolog, subfamily C, member 17 |
| | | [Source:HGNC Symbol;Acc:25556] |
| MYEF2 | MEF-2; FLJ11213; | myelin expression factor 2 [Source:HGNC |
| | KIAA1341; | Symbol;Acc:17940] |
| | HsT18564 | |
| ESRP1 | FLJ20171 | epithelial splicing regulatory protein 1 [Source:HGNC |
| | | Symbol;Acc:25966] |
| HNRNPL | | heterogeneous nuclear ribonucleoprotein L |
| | | [Source:HGNC Symbol;Acc:5045] |
| SNRNP70 | U1-70K; Snpl | small nuclear ribonucleoprotein 70kDa (U1) |
| | | [Source:HGNC Symbol;Acc:11150] |
| SFRS16 | SWAP2; CLASP | splicing factor, arginine/serine-rich 16 [Source:HGNC |
| | | Symbol;Acc:17731] |
| TRMT1 | FLJ20244 | TRM1 tRNA methyltransferase 1 homolog (S. |
| | | cerevisiae) [Source:HGNC Symbol;Acc:25980] |
| NOVA2 | ANOVA | neuro-oncological ventral antigen 2 [Source:HGNC |
| | | Symbol;Acc:7887] |
| SF4 | F23858; | splicing factor 4 [Source:HGNC Symbol;Acc:18643] |
| | DKFZp434E2216; | |
| | RBP | |
| ZC3HAV1 | ZAP; FLB6421; | zinc finger CCCH-type, antiviral 1 [Source:HGNC |
| | FLJ13288; | Symbol;Acc:23721] |
| | MGC48898; | |
| | ZC3HDC2; | |
| | ZC3H2; PARP13 | |
| EIF3B | PRT1; eIF3b | eukaryotic translation initiation factor 3, subunit B |
| | | [Source:HGNC Symbol;Acc:3280] |
| RBM28 | FLJ10377 | RNA binding motif protein 28 [Source:HGNC |
| | | Symbol;Acc:21863] |
| LSM5 | YER146W | LSM5 homolog, U6 small nuclear RNA associated (S. |
| | | cerevisiae) [Source:HGNC Symbol;Acc:17162] |
| EIF4H | WSCR1; | eukaryotic translation initiation factor 4H |
| | KIAA0038 | [Source:HGNC Symbol;Acc:12741] |
| ELAVL2 | HuB; HEL-N1 | ELAV (embryonic lethal, abnormal vision, Drosophila)- |
| | | like 2 (Hu antigen B) [Source:HGNC Symbol;Acc:3313] |
| FUBP3 | | far upstream element (FUSE) binding protein 3 |
| | | [Source:HGNC Symbol;Acc:4005] |
| CPEB3 | KIAA0940 | cytoplasmic polyadenylation element binding protein 3 |
| | | [Source:HGNC Symbol;Acc:21746] |
| LARP4B | | La ribonucleoprotein domain family, member 4B |
| | | [Source:HGNC Symbol;Acc:28987] |
| SUPT6H | KIAA0162; | suppressor of Ty 6 homolog (S. cerevisiae) |
| | SPT6H | [Source:HGNC Symbol;Acc:11470] |
| PPARGC1A | PGC1; PGC1A | peroxisome proliferator-activated receptor gamma, |
| | | coactivator 1 alpha [Source:HGNC Symbol;Acc:9237] |
| CPSF6 | CFIM; HPBRII-4; | microRNA 1279 [Source:HGNC Symbol;Acc:35357] |
| | HPBRII-7; | |
| | CFIM68 | |
| KRR1 | RIP-1 | KRR1, small subunit (SSU) processome component, |
| | | homolog (yeast) [Source:HGNC Symbol;Acc:5176] |
| SFRS9 | SRp30c | splicing factor, arginine/serine-rich 9 [Source:HGNC |
| | | Symbol;Acc:10791] |
| SFRS3 | SRp20 | splicing factor, arginine/serine-rich 3 [Source:HGNC |
| | | Symbol;Acc:10785] |
| RBM24 | FLJ30829; | RNA binding motif protein 24 [Source:HGNC |
| | dJ259A10.1 | Symbol;Acc:21539] |
| KHDRBS2 | SLM1; SLM-1; | KH domain containing, RNA binding, signal |
| | MGC26664 | transduction associated 2 [Source:HGNC |
| | | Symbol;Acc:18114] |
| QKI | QK3 | quaking homolog, KH domain RNA binding (mouse) |
| | | [Source:HGNC Symbol;Acc:21100] |
| CPEB4 | KIAA1673 | cytoplasmic polyadenylation element binding protein 4 |
| | | [Source:HGNC Symbol;Acc:21747] |
| FXR1 | | fragile X mental retardation, autosomal homolog 1 |
| | | [Source:HGNC Symbol;Acc:4023] |
| NCL | C23 | nucleolin [Source:HGNC Symbol;Acc:7667] |
| | | Pre-mRNA branch site protein p14 (SF3B 14 kDa |
| | | subunit) [Source:UniProtKB/Swiss-Prot;Acc:Q9Y3B4] |
| HDLBP | HBP | high density lipoprotein binding protein [Source:HGNC |
| | | Symbol;Acc:4857] |
| SFRS7 | 9G8; ZCRB2; | splicing factor, arginine/serine-rich 7, 35kDa |
| | HSSG1; AAG3; | [Source:HGNC Symbol;Acc:10789] |
| | RBM37; | |
| | ZCCHC20 | |
| PNO1 | | partner of NOB1 homolog (S. cerevisiae) |
| | | [Source: HGNC Symbol;Acc:32790] |
| TIA1 | | TIA1 cytotoxic granule-associated RNA binding protein |
| | | [Source:HGNC Symbol;Acc:11802] |
| SFRS4 | 5RP75 | splicing factor, arginine/serine-rich 4 [Source:HGNC |
| | | Symbol;Acc:10786] |
| SFPQ | PSF | splicing factor proline/glutamine-rich (polypyrimidine |
| | | tract binding protein associated) [Source:HGNC |
| | | Symbol;Acc:10774] |
| SFRS11 | p54; NET2 | splicing factor, arginine/serine-rich 11 [Source:HGNC |
| | | Symbol;Acc:10782] |
| PRPF3 | Prp3; HPRP3 | PRP3 pre-mRNA processing factor 3 homolog (S. |
| | | cerevisiae) [Source:HGNC Symbol;Acc:17348] |
| PTBP2 | brPTB; nPTB; | polypyrimidine tract binding protein 2 [Source:HGNC |
| | PTB; PTBLP | Symbol;Acc:17662] |
| ROD1 | PTBP3 | ROD1 regulator of differentiation 1 (S. pombe) |
| | | [Source:HGNC Symbol;Acc:10253] |
| RBM18 | MGC2734 | RNA binding motif protein 18 [Source:HGNC |
| | | Symbol;Acc:28413] |
| C14orf156 | DC50 | SRA stem-loop-interacting RNA-binding protein, |
| | | mitochondrial Precursor [Source:UniProtKB/Swiss- |
| | | Prot;Acc:Q9GZT3] |
| RBM25 | S164; fSAP94; | RNA binding motif protein 25 [Source:HGNC |
| | NET52; Snu71 | Symbol;Acc:23244] |
| ENOX1 | FLJ10094; PIG38; | ecto-NOX disulfide-thiol exchanger 1 [Source:HGNC |
| | CNOX; cCNOX | Symbol;Acc:25474] |
| TARDBP | TDP-43; ALS10 | TAR DNA binding protein [Source:HGNC |
| | | Symbol;Acc:11571] |
| AKAP1 | AKAP121; | A kinase (PRKA) anchor protein 1 [Source:HGNC |
| | AKAP149; | Symbol;Acc:367] |
| | SAKAP84; S- | |
| | AKAP84; | |
| | AKAP84 | |
| PSPC1 | PSP1; FLJ10955 | paraspeckle component 1 [Source:HGNC |
| | | Symbol;Acc:20320] |
| ZCCHC17 | PS1D; HSPC251; | zinc finger, CCHC domain containing 17 [Source:HGNC |
| | pNO40 | Symbol;Acc:30246] |
| KHDRBS1 | Sam68; p62; | KH domain containing, RNA binding, signal |
| | FLJ34027 | transduction associated 1 [Source:HGNC |
| | | Symbol;Acc:18116] |
| ZC3H7A | HSPC055; | zinc finger CCCH-type containing 7A [Source:HGNC |
| | FLJ20318 | Symbol;Acc:30959] |
| HNRNPA2B1 | | heterogeneous nuclear ribonucleoprotein A2/B1 |
| | | [Source:HGNC Symbol;Acc:5033] |
| BICC1 | | bicaudal C homolog 1 (Drosophila) [Source:HGNC |
| | | Symbol;Acc:19351] |
| RBM19 | DKFZp586F1023; | RNA binding motif protein 19 [Source:HGNC |
| | KIAA0682 | Symbol;Acc:29098] |
| ZC3H13 | DKFZp434D1812 | zinc finger CCCH-type containing 13 [Source:HGNC |
| | | Symbol;Acc:20368] |
| SFRS6 | SRP55; B52 | splicing factor, arginine/serine-rich 6 [Source:HGNC |
| | | Symbol;Acc:10788] |
| RNF113A | RNF113; | ring finger protein 113A [Source:HGNC |
| | Cwc24 | Symbol;Acc:12974] |
| SNRPD2 | Sm-D2 | small nuclear ribonucleoprotein D2 polypeptide 16.5kDa |
| | | [Source:HGNC Symbol;Acc:11159] |
| SNRPB | COD; SmB/SmB′; | small nuclear ribonucleoprotein polypeptides B and B1 |
| | Sm-B/B′; snRNP-B | [Source:HGNC Symbol;Acc:11153] |
| SNRPB2 | Msl1 | small nuclear ribonucleoprotein polypeptide B″ |
| | | [Source:HGNC Symbol;Acc:11155] |
| HNRNPR | hnRNP-R | heterogeneous nuclear ribonucleoprotein R |
| | | [Source:HGNC Symbol;Acc:5047] |
| RALY | P542; HNRPCL2 | RNA binding protein, autoantigenic (hnRNP-associated |
| | | with lethal yellow homolog (mouse)) [Source:HGNC |
| | | Symbol;Acc:15921] |
| RBM42 | MGC10433 | RNA binding motif protein 42 [Source:HGNC |
| | | Symbol;Acc:28117] |
| HNRNPH2 | hnRNPH′; FTP3; | heterogeneous nuclear ribonucleoprotein H2 (H′) |
| | HNRPH′ | [Source:HGNC Symbol;Acc:5042] |
| ZFP36 | RNF162A; TIS11; | zinc finger protein 36, C3H type, homolog (mouse) |
| | G0524; TTP; | [Source:HGNC Symbol;Acc:12862] |
| | NUP475 | |
| NAA38 | YJR022W | N(alpha)-acetyltransferase 38, NatC auxiliary subunit |
| | | [Source:HGNC Symbol;Acc:20471] |
| SNRPN | SMN; SM-D; | small nuclear ribonucleoprotein polypeptide N |
| | HCERN3; | [Source:HGNC Symbol;Acc:11164] |
| | SNRNP-N; | |
| | SNURF-SNRPN; | |
| | RT-LI | |
| FXR2 | | fragile X mental retardation, autosomal homolog 2 |
| | | [Source:HGNC Symbol;Acc:4024] |
| SAFB2 | KIAA0138 | scaffold attachment factor B2 [Source:HGNC |
| | | Symbol;Acc:21605] |
| LSM7 | YNL147W | LSM7 homolog, U6 small nuclear RNA associated (S. |
| | | cerevisiae) [Source:HGNC Symbol;Acc:20470] |
| LSM4 | YER112W | LSM4 homolog, U6 small nuclear RNA associated (S. |
| | | cerevisiae) [Source:HGNC Symbol;Acc:17259] |
| ZC3H4 | KIAA1064 | zinc finger CCCH-type containing 4 [Source:HGNC |
| | | Symbol;Acc:17808] |
| EIF3G | eIF3-delta; eIF3- | eukaryotic translation initiation factor 3, subunit G |
| | p44; eIF3g | [Source:HGNC Symbol;Acc:3274] |
| DKC1 | XAP101; dyskerin; | small nucleolar RNA, H/ACA box 56 [Source:HGNC |
| | NAP57; NOLA4 | Symbol;Acc:32650] |
| PPIL4 | | peptidylprolyl isomerase (cyclophilin)-like 4 |
| | | [Source:HGNC Symbol;Acc:15702] |
| RBM39 | CC1.3; HCC1; | RNA binding motif protein 39 [Source:HGNC |
| | CAPER; fSAP59 | Symbol;Acc:15923] |
| ANKHD1 | MASK; F1120288; | ankyrin repeat and KH domain containing 1 |
| | FLJ11979; | [Source:HGNC Symbol;Acc:24714] |
| | FLJ10042; | |
| | FLJ14127; | |
| | KIAA1085 | |
| KHDRBS3 | T-STAR; Etle; | KH domain containing, RNA binding, signal |
| | etoile; SALP; | transduction associated 3 [Source:HGNC |
| | SLM2; SLM-2 | Symbol;Acc:18117] |
| RBM8A | ZNRP; BOV-1A; | RNA binding motif protein 8A [Source:HGNC |
| | BOV-1B; BOV- | Symbol;Acc:9905] |
| | 1C; RBM8B; Y14 | |
| LIN28 | | lin-28 homolog (C. elegans) [Source:HGNC |
| | | Symbol;Acc:15986] |
| GRSF1 | | G-rich RNA sequence binding factor 1 [Source:HGNC |
| | | Symbol;Acc:4610] |
| ANKRD17 | GTAR; | ankyrin repeat domain 17 [Source:HGNC |
| | KIAA0697; | Symbol;Acc:23575] |
| | FLJ22206; NY- | |
| | BR-16 | |
| UNK | KIAA1753 | unkempt homolog (Drosophila) [Source:HGNC |
| | | Symbol;Acc:29369] |
| NIP7 | CGI-37; | nuclear import 7 homolog (S. cerevisiae) [Source:HGNC |
| | FLJ10296; | Symbol;Acc:24328] |
| | HSPC031; KD93 | |
| TOE1 | | target of EGR1, member 1 (nuclear) [Source:HGNC |
| | | Symbol;Acc:15954] |
| RBM38 | HSRNASEB; | RNA binding motif protein 38 [Source:HGNC |
| | SEB4D; seb4B; | Symbol;Acc:15818] |
| | dJ800J21.2 | |
| SRRM1 | SRM160; POP101; | serine/arginine repetitive matrix 1 [Source:HGNC |
| | MGC39488 | Symbol;Acc:16638] |
| MKRN1 | RNF61 | makorin ring finger protein 1 [Source:HGNC |
| | | Symbol;Acc:7112] |
| EIF2S1 | EIF-2a1pha; EIF2A | eukaryotic translation initiation factor 2, subunit 1 alpha, |
| | | 35kDa [Source:HGNC Symbol;Acc:3265] |
| THUMPD3 | DKFZP434F091 | THUMP domain containing 3 [Source:HGNC |
| | | Symbol;Acc:24493] |
| RBMX2 | CGI-79 | RNA binding motif protein, X-linked 2 [Source:HGNC |
| | | Symbol;Acc:24282] |
| PUM1 | PUMH1; | pumilio homolog 1 (Drosophila) [Source:HGNC |
| | KIAA0099 | Symbol;Acc:14957] |
| MSI1 | | musashi homolog 1 (Drosophila) [Source:HGNC |
| | | Symbol;Acc:7330] |
| SYNCRIP | NSAP1; GRY- | synaptotagmin binding, cytoplasmic RNA interacting |
| | RBP; dJ3J17.2; | protein [Source:HGNC Symbol;Acc:16918] |
| | HNRPQ1; hnRNP-Q | |
| ZC3H10 | FLJ14451 | zinc finger CCCH-type containing 10 [Source:HGNC |
| | | Symbol;Acc:25893] |
| HNRNPA1 | hnRNPA1; | heterogeneous nuclear ribonucleoprotein A1 |
| | hnRNP-Al | [Source:HGNC Symbol;Acc:5031] |
| RC3H1 | KIAA2025; | ring finger and CCCH-type zinc finger domains 1 |
| | roquin; RPS- | [Source:HGNC Symbol;Acc:29434] |
| | 1198E17.5; | |
| | RNF198 | |
| IGF2BP3 | IMP-3; CT98 | insulin-like growth factor 2 mRNA binding protein 3 |
| | | [Source:HGNC Symbol;Acc:28868] |
| NUPL2 | NLP_1; CG1; | nucleoporin like 2 [Source:HGNC Symbol;Acc:17010] |
| | hCG1; | |
| | H_RG271G13.9 | |
| SFRS1 | ASF; SF2; | splicing factor, arginine/serine-rich 1 [Source:HGNC |
| | SRp30a; SF2p33; | Symbol;Acc:10780] |
| | MGC5228 | |
| TRA2B | Htra2-beta | transformer 2 beta homolog (Drosophila) [Source:HGNC |
| | | Symbol;Acc:10781] |
| CPEB2 | | cytoplasmic polyadenylation element binding protein 2 |
| | | [Source:HGNC Symbol;Acc:21745] |
| ALKBH8 | MGC10235 | alkB, alkylation repair homolog 8 (E. coli) |
| | | [Source:HGNC Symbol;Acc:25189] |
| SLTM | Met; FLJ13213 | SAFB-like, transcription modulator [Source:HGNC |
| | | Symbol;Acc:20709] |
| PNPT1 | PNPase; OLD35; | polyribonucleotide nucleotidyltransferase 1 |
| | old-35 | [Source:HGNC Symbol;Acc:23166] |
| THUMPD2 | MGC2454 | THUMP domain containing 2 [Source:HGNC |
| | | Symbol;Acc:14890] |
| ASCC1 | CGI-18; ASC1p50; | activating signal cointegrator 1 complex subunit 1 |
| | Em:AC022392.3 | [Source:HGNC Symbol;Acc:24268] |
| SSB | LARP3; La | Sjogren syndrome antigen B (autoantigen La) |
| | | [Source:HGNC Symbol;Acc:11316] |
| HNRNPD | | heterogeneous nuclear ribonucleoprotein D (AU-rich |
| | | element RNA binding protein 1, 37kDa) [Source:HGNC |
| | | Symbol;Acc:5036] |
| LARP1B | FLJ10378; | La ribonucleoprotein domain family, member 1B |
| | DKFZp434K245; | [Source:HGNC Symbol;Acc:24704] |
| | DKFZp686E0316 | |
| G3BP2 | KIAA0660 | GTPase activating protein (5H3 domain) binding protein |
| | | 2 [Source:HGNC Symbol;Acc:30291] |
| ZCRB1 | MADP-1; | zinc finger CCHC-type and RNA binding motif 1 |
| | MADP1; RBM36; | [Source:HGNC Symbol;Acc:29620] |
| | ZCCHC19 | |
| SNRPF | Sm-F | small nuclear ribonucleoprotein polypeptide F |
| | | [Source:HGNC Symbol;Acc:11162] |
| | | Heterogeneous nuclear ribonucleoprotein A1-like protein |
| | | 2 (hnRNP core protein A1-like protein 2) |
| | | [Source:UniProtKB/Swiss-Prot;Acc:Q32P51] |
| SETD1B | KIAA1076; Set1B; | SET domain containing 1B [Source:HGNC |
| | KMT2G | Symbol;Acc:29187] |
| RBM26 | PRO1777; 5E70-2; | RNA binding motif protein 26 [Source:HGNC |
| | FLJ20957; | Symbol;Acc:20327] |
| | ZC3H17; ARRS2 | |
| MBNL2 | MBLL; MBLL39 | muscleblind-like 2 (Drosophila) [Source:HGNC |
| | | Symbol;Acc:16746] |
| RNF113B | RNF161 | ring finger protein 113B [Source:HGNC |
| | | Symbol;Acc:17267] |
| NOVA1 | | neuro-oncological ventral antigen 1 [Source:HGNC |
| | | Symbol;Acc:7886] |
| BRUNOL6 | | bruno-like 6, RNA binding protein (Drosophila) |
| | | [Source:HGNC Symbol;Acc:14059] |
| DUS3L | DUS3; FLJ13896 | dihydrouridine synthase 3-like (S. cerevisiae) |
| | | [Source:HGNC Symbol;Acc:26920] |
| SF3B4 | SAP49; SF3b49; | splicing factor 3b, subunit 4, 49kDa [Source:HGNC |
| | Hsh49 | Symbol;Acc:10771] |
| LGTN | | ligatin [Source:HGNC Symbol;Acc:6583] |
| HNRPLL | | heterogeneous nuclear ribonucleoprotein L-like |
| | | [Source:HGNC Symbol;Acc:25127] |
| SNRPG | Sm-G | small nuclear ribonucleoprotein polypeptide G |
| | | [Source:HGNC Symbol;Acc:11163] |
| ZC3H8 | Fliz1 | zinc finger CCCH-type containing 8 [Source:HGNC |
| | | Symbol;Acc:30941] |
| RBMS3 | | RNA binding motif, single stranded interacting protein |
| | | [Source:HGNC Symbol;Acc:13427] |
| G3BP1 | HDH-VIII; G3BP | GTPase activating protein (SH3 domain) binding protein |
| | | 1 [Source:HGNC Symbol;Acc:30292] |
| NONO | NRB54; NMT55; | non-POU domain containing, octamer-binding |
| | P54NRB; P54 | [Source:HGNC Symbol;Acc:7871] |
| RBMX | RNMX; hnRNP-G | RNA binding motif protein, X-linked [Source:HGNC |
| | | Symbol;Acc:9910] |
| A1CF | ACF; ASP; | APOBEC1 complementation factor [Source:HGNC |
| | ACF64; ACF65; | Symbol;Acc:24086] |
| | APOBEC1CF | |
| PPRC1 | PRC; KIAA0595; | peroxisome proliferator-activated receptor gamma, |
| | MGC74642 | coactivator-related 1 [Source:HGNC |
| | | Symbol;Acc:30025] |
| PDCD11 | KIAA0185; ALG- | programmed cell death 11 [Source:HGNC |
| | 4 | Symbol;Acc:13408] |
| TUT1 | FLJ22347; | terminal uridylyl transferase 1, U6 snRNA-specific |
| | FLJ22267; | [Source:HGNC Symbol;Acc:26184] |
| | FLJ21850; | |
| | PAPD2; TUTase | |
| CUGBP1 | | CUG triplet repeat, RNA binding protein 1 |
| | | [Source:HGNC Symbol;Acc:2549] |
| RPS3 | FLJ26283; | ribosomal protein S3 [Source:HGNC |
| | FLJ27450; | Symbol;Acc:10420] |
| | MGC87870 | |
| ZC3H12C | KIAA1726; | zinc finger CCCH-type containing 12C [Source:HGNC |
| | MCPIP3 | Symbol;Acc:29362] |
| CPSF7 | FLJ12529 | cleavage and polyadenylation specific factor 7, 59kDa |
| | | [Source:HGNC Symbol;Acc:30098] |
| YTHDF1 | FLJ20391 | YTH domain family, member 1 [Source:HGNC |
| | | Symbol;Acc:15867] |
| PABPC3 | PABP3; tPABP | poly(A) binding protein, cytoplasmic 3 [Source:HGNC |
| | | Symbol;Acc:8556] |
| TIAL1 | TIAR | TIA1 cytotoxic granule-associated RNA binding protein- |
| | | like 1 [Source:HGNC Symbol;Acc:11804] |
| RBM46 | MGC27016; CT68 | RNA binding motif protein 46 [Source:HGNC |
| | | Symbol;Acc:28401] |
| UHMK1 | KIS; Kist | U2AF homology motif (UHM) kinase 1 [Source:HGNC |
| | | Symbol;Acc:19683] |
| BOLL | BOULE | bol, boule-like (Drosophila) [Source:HGNC |
| | | Symbol;Acc:14273] |
| ZFP36L2 | ERF2; RNF162C; | zinc finger protein 36, C3H type-like 2 [Source:HGNC |
| | TIS11D | Symbol;Acc:1108] |
| PAN3 | | PAN3 poly(A) specific ribonuclease subunit homolog (S. |
| | | cerevisiae) [Source:HGNC Symbol;Acc:29991] |
| MBNL1 | KIAA0428; | muscleblind-like (Drosophila) [Source:HGNC |
| | EXP42; EXP40; | Symbol;Acc:6923] |
| | EXP35; EXP | |
| HNRPDL | JKTBP; laAUF1 | heterogeneous nuclear ribonucleoprotein D-like |
| | | [Source:HGNC Symbol;Acc:5037] |
| CARHSP1 | CRHSP-24; | calcium regulated heat stable protein 1, 24kDa |
| | CSDC1 | [Source:HGNC Symbol;Acc:17150] |
| RBMS1 | SCR2; MSSP-1; | RNA binding motif, single stranded interacting protein 1 |
| | MSSP-2; MSSP-3; | [Source:HGNC Symbol;Acc:9907] |
| | YC1; HCC-4; | |
| | DKFZp564H0764 | |
| SFRS12 | DKFZp564B176; | splicing factor, arginine/serine-rich 12 [Source:HGNC |
| | SRrp86; SRrp508 | Symbol;Acc:17882] |
| MSI2 | | musashi homolog 2 (Drosophila) [Source:HGNC |
| | | Symbol;Acc:18585] |
| SFRS13B | SRrp35; SFRS19 | splicing factor, arginine/serine-rich 13B [Source:HGNC |
| | | Symbol;Acc:21220] |
| C4orf23 | FLJ35725 | Probable tRNA (uracil-O(2)-)-methyltransferase (EC |
| | | 2.1.1.n2) [Source:UniProtKB/Swiss-Prot;Acc:Q8IYL2] |
| MKI67IP | Nopp34; NIFK | MKI67 (FHA domain) interacting nucleolar |
| | | phosphoprotein [Source:HGNC Symbol;Acc:17838] |
| LARP1 | LARP; KIAA0731; | La ribonucleoprotein domain family, member 1 |
| | MGC19556 | [Source:HGNC Symbol;Acc:29531] |
| RBM45 | DRB1; FLJ44612 | RNA binding motif protein 45 [Source:HGNC |
| | | Symbol;Acc:24468] |
| PPARGC1B | PERC; PGC1B | peroxisome proliferator-activated receptor gamma, |
| | | coactivator 1 beta [Source:HGNC Symbol;Acc:30022] |
| LSM11 | FLJ38273 | LSM11, U7 small nuclear RNA associated |
| | | [Source:HGNC Symbol;Acc:30860] |
| SFRS15 | KIAA1172; | splicing factor, arginine/serine-rich 15 [Source:HGNC |
| | DKFZp434E098; | Symbol;Acc:19304] |
| | SRA4; SCAF4 | |
| RBPMS | HERMES | RNA binding protein with multiple splicing |
| | | [Source:HGNC Symbol;Acc:19097] |
| ZC3H18 | NHN1 | zinc finger CCCH-type containing 18 [Source:HGNC |
| | | Symbol;Acc:25091] |
| SYNJ1 | INPP5G | synaptojanin 1 [Source:HGNC Symbol;Acc:11503] |
| IGF2BP1 | IMP-1; ZBP1 | insulin-like growth factor 2 mRNA binding protein 1 |
| | | [Source:HGNC Symbol;Acc:28866] |
| TNRC4 | | trinucleotide repeat containing 4 [Source:HGNC |
| | | Symbol;Acc:11967] |
| U2AF1 | U2AF35; | U2 small nuclear RNA auxiliary factor 1 [Source:HGNC |
| | RNU2AF1; RN | Symbol;Acc:12453] |
| SAFB | HET; SAFB1 | scaffold attachment factor B [Source:HGNC |
| | | Symbol;Acc:10520] |
| CPSF4 | NAR; CPSF30 | cleavage and polyadenylation specific factor 4, 30kDa |
| | | [Source:HGNC Symbol;Acc:2327] |
| BRUNOL5 | | bruno-like 5, RNA binding protein (Drosophila) |
| | | [Source:HGNC Symbol;Acc:14058] |
| U2AF1L4 | MGC33901; | U2 small nuclear RNA auxiliary factor 1-like 4 |
| | U2af26 | [Source:HGNC Symbol;Acc:23020] |
| SFRS2 | SC-35; SC35; | splicing factor, arginine/serine-rich 2 [Source:HGNC |
| | PR264; SFRS2A | Symbol;Acc:10783] |
| LARP4 | PP13296 | La ribonucleoprotein domain family, member 4 |
| | | [Source:HGNC Symbol;Acc:24320] |
| RAVER1 | KIAA1978 | ribonucleoprotein, PTB-binding 1 [Source:HGNC |
| | | Symbol;Acc:30296] |
| ELAVL4 | PNEM | ELAV (embryonic lethal, abnormal vision, Drosophila)- |
| | | like 4 (Hu antigen D) [Source:HGNC Symbol;Acc:3315] |
| RAVER2 | KIAA1579; | ribonucleoprotein, PTB-binding 2 [Source:HGNC |
| | FLJ10770 | Symbol;Acc:25577] |
| FUBP1 | FBP | far upstream element (FUSE) binding protein 1 |
| | | [Source:HGNC Symbol;Acc:4004] |
| RBM15 | OTT; OTT1 | RNA binding motif protein 15 [Source:HGNC |
| | | Symbol;Acc:14959] |
| DHX57 | DDX57 | DEAH (Asp-Glu-Ala-Asp/His) box polypeptide 57 |
| | | [Source:HGNC Symbol;Acc:20086] |
| TDRD10 | DKFZp434M202 | tudor domain containing 10 [Source:HGNC |
| | | Symbol;Acc:25316] |
| TIPARP | DKFZP434J214; | TCDD-inducible poly(ADP-ribose) polymerase |
| | DKFZp686N0351; | [Source:HGNC Symbol;Acc:23696] |
| | DDF1; PARP7; | |
| | PARP-7; PARP-1; | |
| | pART14; RM1 | |
| RBM47 | F1120273; NET18 | RNA binding motif protein 47 [Source:HGNC |
| | | Symbol;Acc:30358] |
| SR140 | | U2-associated protein SR140 (140 kDa Ser/Arg-rich |
| | | domain protein) [Source:UniProtKB/Swiss- |
| | | Prot;Acc:O15042] |
| TTC14 | FLJ00166; | tetratricopeptide repeat domain 14 [Source:HGNC |
| | KIAA1980 | Symbol;Acc:24697] |
| LSM6 | YDR378C | LSM6 homolog, U6 small nuclear RNA associated (S. |
| | | cerevisiae) [Source:HGNC Symbol;Acc:17017] |
| TRA2A | htra-2-alpha; tra2a | transformer 2 alpha homolog (Drosophila) |
| | | [Source:HGNC Symbol;Acc:16645] |
| HNRNPK | CSBP; TUNP | microRNA 7-1 [Source:HGNC Symbol;Acc:31638] |
| ENOX2 | APK1; tNOX | ecto-NOX disulfide-thiol exchanger 2 [Source:HGNC |
| | | Symbol;Acc:2259] |
| LARP6 | acheron; FLJ11196 | La ribonucleoprotein domain family, member 6 |
| | | [Source:HGNC Symbol;Acc:24012] |
| KIAA0430 | LKAP | KIAA0430 [Source:HGNC Symbol;Acc:29562] |
| RBPMS2 | | RNA binding protein with multiple splicing 2 |
| | | [Source:HGNC Symbol;Acc:19098] |
| SNRPD1 | HsT2456; Sm-D1 | small nuclear ribonucleoprotein D1 polypeptide 16kDa |
| | | [Source:HGNC Symbol;Acc:11158] |
| SAMD14 | FLJ36890 | sterile alpha motif domain containing 14 [Source:HGNC |
| | | Symbol;Acc:27312] |
| | | Fox-1 homolog C [Source:UniProtKB/Swiss- |
| | | Prot;Acc:A6NFN3] |
| POLR2G | hRPB19; hsRPB7; | polymerase (RNA) II (DNA directed) polypeptide G |
| | RPB7 | [Source:HGNC Symbol;Acc:9194] |
| SF1 | ZFM1 | splicing factor 1 [Source:HGNC Symbol;Acc:12950] |
| HNRNPH1 | hnRNPH | heterogeneous nuclear ribonucleoprotein H1 (H) |
| | | [Source:HGNC Symbol;Acc:5041] |
| ZRSR2 | U2AF1-RS2; | zinc finger (CCCH type), RNA-binding motif and |
| | URP | serine/arginine rich 2 [Source:HGNC |
| | | Symbol;Acc:23019] |
| PCBP1 | HNRPE1; hnRNP- | poly(rC) binding protein 1 [Source:HGNC |
| | E1; HNRPX; | Symbol;Acc:8647] |
| | hnRNP-X | |
| RBMY1F | MGC33094 | RNA binding motif protein, Y-linked, family 1, member |
| | | F [Source:HGNC Symbol;Acc:23974] |
| HNRNPF | | heterogeneous nuclear ribonucleoprotein F |
| | | [Source:HGNC Symbol;Acc:5039] |
| HNRNPA3 | | heterogeneous nuclear ribonucleoprotein A3 |
| | | [Source:HGNC Symbol;Acc:24941] |
| RBMXL2 | HNRNPG-T; | RNA binding motif protein, X-linked-like 2 |
| | HNRPGT | [Source:HGNC Symbol;Acc:17886] |
| LSM3 | YLR438C; SMX4; | LSM3 homolog, U6 small nuclear RNA associated (S. |
| | U552 | cerevisiae) [Source:HGNC Symbol;Acc:17874] |
| NCBP2L | | nuclear cap binding protein subunit 2-like |
| | | [Source:HGNC Symbol;Acc:31795] |
| CSDC2 | PIPPin | cold shock domain containing C2, RNA binding |
| | | [Source:HGNC Symbol;Acc:30359] |
| TAF15 | hTAFII68; RBP56; | TAF15 RNA polymerase II, TATA box binding protein |
| | Np13 | (TBP)-associated factor, 68kDa [Source:HGNC |
| | | Symbol;Acc:11547] |
| RBM4B | MGC10871; | RNA binding motif protein 4B [Source:HGNC |
| | ZCCHC15; | |
| | RBM4L; ZCRB3B | Symbol;Acc:28842] |
| RBM4 | LARK; RBM4A; | RNA binding motif protein 4 [Source:HGNC |
| | ZCRB3A; | Symbol;Acc:9901] |
| | ZCCHC21 | |
| LARP7 | HDCMA18P; | La ribonucleoprotein domain family, member 7 |
| | PIP7S; | [Source:HGNC Symbol;Acc:24912] |
| | DKFZP564K112 | |
| PABPC5 | PABP5 | poly(A) binding protein, cytoplasmic 5 [Source:HGNC |
| | | Symbol;Acc:13629] |
| LSM1 | CASM; YJL124C | LSM1 homolog, U6 small nuclear RNA associated (S. |
| | | cerevisiae) [Source:HGNC Symbol;Acc:20472] |
| RBMXL3 | FLJ40249 | RNA binding motif protein, X-linked-like 3 |
| | | [Source:HGNC Symbol;Acc:26859] |
| MEX3C | FLJ38871; | mex-3 homolog C (C. elegans) [Source:HGNC |
| | RNF194 | Symbol;Acc:28040] |
| RBM44 | FLJ40411 | RNA binding motif protein 44 [Source:HGNC |
| | | Symbol;Acc:24756] |
| CSTF2T | DKFZp434C1013; | cleavage stimulation factor, 3′pre-RNA, subunit 2, |
| | KIAA0689; CstF- | 64kDa, tau variant [Source:HGNC Symbol;Acc:17086] |
| | 64T | |
| HNRNPA0 | hnRNPA0 | heterogeneous nuclear ribonucleoprotein A0 |
| | | [Source:HGNC Symbol;Acc:5030] |
| ZC3H12D | dJ281H8.1; | zinc finger CCCH-type containing 12D [Source:HGNC |
| | MCPIP4 | Symbol;Acc:21175] |
| SAMD4B | FLJ10211; | sterile alpha motif domain containing 4B [Source:HGNC |
| | MGC99832; | Symbol;Acc:25492] |
| | SMGB; h5maug2 | |
| HNRNPCL1 | | heterogeneous nuclear ribonucleoprotein C-like 1 |
| | | [Source:HGNC Symbol;Acc:29295] |
| MKRN3 | RNF63; ZFP127; | makorin ring finger protein 3 [Source:HGNC |
| | MGC88288 | Symbol;Acc:7114] |
| RBM15B | HUMAGCGB; | RNA binding motif protein 15B [Source:HGNC |
| | OTT3 | Symbol;Acc:24303] |
| PUF60 | FIR; SIABBP1; | poly-U binding splicing factor 60KDa [Source:HGNC |
| | RoBPI | Symbol;Acc:17042] |
| TRNAU1AP | SECP43; | tRNA selenocysteine 1 associated protein 1 |
| | FLJ20503 | [Source:HGNC Symbol;Acc:30813] |
| SFRS2B | SRP46 | splicing factor, arginine/serine-rich 2B [Source:HGNC |
| | | Symbol;Acc:16988] |
| MEX3D | Tino; KIAA2031; | mex-3 homolog D (C. elegans) [Source:HGNC |
| | OK/SW-c1.4; | Symbol;Acc:16734] |
| | RNF193 | |
| LSM10 | MGC15749 | LSM10, U7 small nuclear RNA associated |
| | | [Source:HGNC Symbol;Acc:17562] |
| SNRPE | Sm-E | small nuclear ribonucleoprotein polypeptide E |
| | | [Source:HGNC Symbol;Acc:11161] |
| TDRKH | TDRD2 | tudor and KH domain containing [Source:HGNC |
| | | Symbol;Acc:11713] |
| RBM10 | DXS8237E; | RNA binding motif protein 10 [Source:HGNC |
| | KIAA0122; | Symbol;Acc:9896] |
| | GPATC9; | |
| | ZRANB5; | |
| | GPATCH9 | |
| LENG9 | | leukocyte receptor cluster (LRC) member 9 |
| | | [Source:HGNC Symbol;Acc:16306] |
| EWSR1 | EWS | Ewing sarcoma breakpoint region 1 [Source:HGNC |
| | | Symbol;Acc:3508] |
| LSMD1 | MGC14151; | LSM domain containing 1 [Source:HGNC |
| | PFAAP2 | Symbol;Acc:28212] |
| DND1 | MGC34750; | dead end homolog 1 (zebrafish) [Source:HGNC |
| | RBMS4 | Symbol;Acc:23799] |
| MEX3B | DKFZp434J0617; | mex-3 homolog B (C. elegans) [Source:HGNC |
| | RNF195 | Symbol;Acc:25297] |
| PCBP3 | | poly(rC) binding protein 3 [Source:HGNC |
| | | Symbol;Acc:8651] |
| THOC4 | ALY; BEF | THO complex 4 [Source:HGNC Symbol;Acc:19071] |
| RBM12B | | RNA binding motif protein 12B [Source:HGNC |
| | | Symbol;Acc:32310] |
| SNRNP35 | U1SNRNPBP | small nuclear ribonucleoprotein 35kDa (U11/U12) |
| | | [Source:HGNC Symbol;Acc:30852] |
| PABPC1L2B | | poly(A) binding protein, cytoplasmic 1-like 2B |
| | | [Source:HGNC Symbol;Acc:31852] |
| RALYL | HNRPCL3 | RALY RNA binding protein-like [Source:HGNC |
| | | Symbol;Acc:27036] |
| DDX53 | CAGE; CT26 | DEAD (Asp-Glu-Ala-Asp) box polypeptide 53 |
| | | [Source:HGNC Symbol;Acc:20083] |
| RBM33 | DKFZp686F102; | RNA binding motif protein 33 [Source:HGNC |
| | MGC20460; | Symbol;Acc:27223] |
| | DKFZp434D1319 | |
| RBM43 | FLJ45645 | RNA binding motif protein 43 [Source:HGNC |
| | | Symbol;Acc:24790] |
| RBM11 | | RNA binding motif protein 11 [Source:HGNC |
| | | Symbol;Acc:9897] |
| ZFP36L1 | RNF162B; Berg36; | zinc finger protein 36, C3H type-like 1 [Source:HGNC |
| | ERF1; TIS11B; | Symbol;Acc:1107] |
| | cMG1 | |
| YTHDF3 | FLJ31657 | YTH domain family, member 3 [Source:HGNC |
| | | Symbol;Acc:26465] |
| RNPC3 | KIAA1839; | RNA-binding region (RNP1, RRM) containing 3 |
| | FLJ20008; RBM40 | [Source:HGNC Symbol;Acc:18666] |
| PABPC1L2A | | poly(A) binding protein, cytoplasmic 1-like 2A |
| | | [Source:HGNC Symbol;Acc:27989] |
| DAZ3 | | deleted in azoospermia 3 [Source:HGNC |
| | | Symbol;Acc:15965] |
| RDM1 | MGC33977 | RAD52 motif 1 [Source:HGNC Symbol;Acc:19950] |
| LIN28B | FLJ16517; CSDD2 | lin-28 homolog B (C. elegans) [Source:HGNC |
| | | Symbol;Acc:32207] |
| CPSF4L | | cleavage and polyadenylation specific factor 4-like |
| | | [Source:HGNC Symbol;Acc:33632] |
| DAZ1 | SPGY | deleted in azoospermia 1 [Source:HGNC |
| | | Symbol;Acc:2682] |
| ZC3H6 | FLJ41410; | zinc finger CCCH-type containing 6 [Source:HGNC |
| | FLJ45877; | Symbol;Acc:24762] |
| | KIAA2035 | |
| SFRS13A | TASR1; TASR2; | splicing factor, arginine/serine-rich 13A [Source:HGNC |
| | SRp38; SRrp40; | Symbol;Acc: 16713] |
| | SFRS13; FUSIP1; | |
| | FUSIP2 | |
| RBM34 | KIAA0117 | RNA binding motif protein 34 [Source:HGNC |
| | | Symbol;Acc:28965] |
| RRP7A | CGI-96 | ribosomal RNA processing 7 homolog A (S. cerevisiae) |
| | | [Source:HGNC Symbol;Acc:24286] |
| ELAVL3 | HUC; PLE21; | ELAV (embryonic lethal, abnormal vision, Drosophila)- |
| | DKFZp547J036; | like 3 (Hu antigen C) [Source:HGNC Symbol;Acc:3314] |
| | HUCL; | |
| | MGC20653 | |
| C14orf21 | | Pumilio domain-containing protein C14orf21 |
| | | [Source:UniProtKB/Swiss-Prot;Acc:Q86U38] |
| PCBP2 | HNRPE2; hnRNP- | poly(rC) binding protein 2 [Source:HGNC |
| | E2 | Symbol;Acc:8648] |
| ZGPAT | dJ583P15.3; | zinc finger, CCCH-type with G patch domain |
| | MGC44880; | [Source:HGNC Symbol;Acc:15948] |
| | FLJ14972; | |
| | ZC3HDC9; | |
| | ZC3H9; GPATC6; | |
| | GPATCH6; ZIP | |
| HNRNPAB | ABBP1; FLJ40338 | heterogeneous nuclear ribonucleoprotein A/B |
| | | [Source:HGNC Symbol;Acc:5034] |
| NOL8 | FLJ20736; Nop132 | nucleolar protein 8 [Source:HGNC Symbol;Acc:23387] |
| HELZ | KIAA0054; | helicase with zinc finger [Source:HGNC |
| | HUMORF5; | Symbol;Acc:16878] |
| | DHRC | |
| YTHDF2 | HGRG8; NY- | YTH domain family, member 2 [Source:HGNC |
| | REN-2 | Symbol;Acc:31675] |
| SFMBT2 | KIAA1617 | Scm-like with four mbt domains 2 [Source:HGNC |
| | | Symbol;Acc:20256] |
| L3MBTL3 | KIAA1798 | 1(3)mbt-like 3 (Drosophila) [Source:HGNC |
| | | Symbol;Acc:23035] |
| MEX3A | | mex-3 homolog A (C. elegans) [Source:HGNC |
| | | Symbol;Acc:33482] |
| RBM20 | | RNA binding motif protein 20 [Source:HGNC |
| | | Symbol;Acc:27424] |
| DPPA5 | Esgl | developmental pluripotency associated 5 [Source:HGNC |
| | | Symbol;Acc:19201] |
| MIR1236 | | microRNA 1236 [Source:HGNC Symbol;Acc:33925] |
| | | U6 snRNA-associated Sm-like protein LSm2 (snRNP |
| | | core Sm-like protein Sm-x5)(Small nuclear ribonuclear |
| | | protein D homolog)(Protein G7b) |
| | | [Source:UniProtKB/Swiss-Prot;Acc:Q9Y333] |
| PPP1R10 | | Serine/threonine-protein phosphatase 1 regulatory |
| | | subunit 10 (Phosphatase 1 nuclear targeting |
| | | subunit)(MHC class I region proline-rich protein |
| | | CAT53)(FB19 protein)(PP1-binding protein of 114 |
| | | kDa)(p99) [Source:UniProtKB/Swiss- |
| | | Prot;Acc:Q96QC0] |
| PRR3 | | Proline-rich protein 3 (MEW class I region proline-rich |
| | | protein CATS 6) [Source:UniProtKB/Swiss- |
| | | Prot;Acc:P79522] |
| PABPN1L | ePABP2 | poly(A) binding protein, nuclear 1-like (cytoplasmic) |
| | | [Source:HGNC Symbol;Acc:37237] |
| RNPSi | | RNA binding protein S1, serine-rich domain |
| | | [Source:HGNC Symbol;Acc:10080] |
| DAZ2 | pDP1678; | deleted in azoospermia 2 [Source:HGNC |
| | MGC126442 | Symbol;Acc:15964] |
| ZRSR1 | | U2 small nuclear ribonucleoprotein auxiliary factor 35 |
| | | kDa subunit-related protein 1 (U2(RNU2) small nuclear |
| | | RNA auxiliary factor 1-like 1)(CCCH type zinc finger, |
| | | RNA-binding motif and serine/arginine rich protein 1) |
| | | [Source:UniProtKB/Swi ss-Prot;Acc : Q15695] |
| RBM16 | KIAA1116 | RNA binding motif protein 16 [Source:HGNC |
| | | Symbol;Acc:20959] |
| RBMXL1 | KAT3 | Putative uncharacterized protein ENSP00000395989 |
| | | [Source:UniProtKB/TrEMBL;Acc:C9JFH9] |
| | | RNA binding motif protein, X-linked-like 1 |
| | | [Source:HGNC Symbol;Acc:25073] |
| CPEB1 | FLJ13203; CPEB | cytoplasmic polyadenylation element binding protein 1 |
| | | [Source:HGNC Symbol;Acc:21744] |
| | | Putative uncharacterized protein ENSP00000388079 |
| | | Fragment [Source:UniProtKB/TrEMBL;Acc:C9JXI7] |
| | | Putative uncharacterized protein ENSP00000383298 |
| | | [Source:UniProtKB/TrEMBL;Acc:C9JCD7] |
| RBMY1J | | RNA binding motif protein, Y-linked, family 1, member |
| | | J [Source:HGNC Symbol;Acc:23917] |
| SNRPEL1 | bA390F4.4 | small nuclear ribonucleoprotein polypeptide E-like 1 |
| | | [Source:HGNC Symbol;Acc:20733] |
| | | Putative uncharacterized protein ENSP00000394145 |
| | | Fragment [Source:UniProtKB/TrEMBL;Acc:C9JK19] |
| MCTS1 | MCT-1 | malignant T cell amplified sequence 1 [Source:HGNC |
| | | Symbol;Acc:23357] |
| PABPC4L | | poly(A) binding protein, cytoplasmic 4-like |
| | | [Source:HGNC Symbol;Acc:31955] |
| RBMY1A1 | YRRM1; YRRM2 | RNA binding motif protein, Y-linked, family 1, member |
| | | A1 [Source:HGNC Symbol;Acc:9912] |
| MKRNP5 | | makorin ring finger protein pseudogene 5 |
| | | [Source:HGNC Symbol;Acc:7115] |
| RBM14 | SIP; SYTIP1; | RNA binding motif protein 14 [Source:HGNC |
| | COAA; | Symbol;Acc:14219] |
| | DKFZp779J0927 | |
| NSUN6 | FLJ23743 | NOP2/Sun domain family, member 6 [Source:HGNC |
| | | Symbol;Acc:23529] |
| UNKL | ZC3HDC5L; | unkempt homolog (Drosophila)-like [Source:HGNC |
| | ZC3H5L; | Symbol;Acc:14184] |
| | FLJ23360 | |
| RBMY1E | | RNA binding motif protein, Y-linked, family 1, member |
| | | E [Source:HGNC Symbol;Acc:23916] |
| HNRNPA1L2 | LOC144983 | heterogeneous nuclear ribonucleoprotein A1-like 2 |
| | | [Source:HGNC Symbol;Acc:27067] |
| RBMY1B | | RNA binding motif protein, Y-linked, family 1, member |
| | | B [Source:HGNC Symbol;Acc:23914] |
| RBMY1D | | RNA binding motif protein, Y-linked, family 1, member |
| | | D [Source:HGNC Symbol;Acc:23915] |
| RBM12 | HRIHFB2091; | RNA binding motif protein 12 [Source:HGNC |
| | KIAA0765; | Symbol;Acc:9898] |
| | SWAN | |
| DAZ4 | | deleted in azoospermia 4 [Source:HGNC |
| | | Symbol;Acc:15966] |
| DGKQ | DAGK; DAGK7 | diacylglycerol kinase, theta 110kDa [Source:HGNC |
| | | Symbol;Acc:2856] |
| RBM17 | SPF45; | RNA binding motif protein 17 [Source:HGNC |
| | MGC14439 | Symbol;Acc:16944] |
| MCM3AP | Map80; | minichromosome maintenance complex component 3 |
| | KIAA0572; GANP | associated protein [Source:HGNC Symbol;Acc:6946] |
| PARN | DAN | poly(A)-specific ribonuclease (deadenylation nuclease) |
| | | [Source:HGNC Symbol;Acc:8609] |
| TLR2 | TIL4; CD282 | toll-like receptor 2 [Source:HGNC Symbol;Acc:11848] |
| PARP10 | FLJ14464 | poly (ADP-ribose) polymerase family, member 10 |
| | | [Source:HGNC Symbol;Acc:25895] |
| MOV10L1 | DJ402G11.8; | Mov1011, Moloney leukemia virus 10-like 1, homolog |
| | DKFZp434B0717 | (mouse) [Source:HGNC Symbol;Acc:7201] |
| CCAR1 | FLJ10590; CARP- | cell division cycle and apoptosis regulator 1 |
| | 1; CARPI | [Source:HGNC Symbol;Acc:24236] |
| LEMD3 | MANI | LEM domain containing 3 [Source:HGNC |
| | | Symbol;Acc:28887] |
| UPF3B | RENT3B; UPF3X; | UPF3 regulator of nonsense transcripts homolog B |
| | HUPF3B | (yeast) [Source:HGNC Symbol;Acc:20439] |
| RCAN2 | ZAKI-4 | regulator of calcineurin 2 [Source:HGNC |
| | | Symbol;Acc:3041] |
| SRRT | Asr2; serrate; | serrate RNA effector molecule homolog (Arabidopsis) |
| | ARS2 | [Source:HGNC Symbol;Acc:24101] |
| ZC3H12A | FLJ23231; | zinc finger CCCH-type containing 12A [Source:HGNC |
| | MCPIP1 | Symbol;Acc:26259] |
| ZC3HAV1L | MGC14289 | zinc finger CCCH-type, antiviral 1-like [Source:HGNC |
| | | Symbol;Acc:22423] |
| PARP14 | KIAA1268; | poly (ADP-ribose) polymerase family, member 14 |
| | pART8 | [Source:HGNC Symbol;Acc:29232] |
| TMEM63A | | transmembrane protein 63A [Source:HGNC |
| | | Symbol;Acc:29118] |
| SLBP | HBP | stem-loop binding protein [Source:HGNC |
| | | Symbol;Acc:10904] |
| GTF3A | | general transcription factor IIIA [Source:HGNC |
| | | Symbol;Acc:4662] |
| NUFIP1 | | nuclear fragile X mental retardation protein interacting |
| | | protein 1 [Source:HGNC Symbol;Acc:8057] |
| SRRM2 | | serine/arginine repetitive matrix 2 [Source:HGNC |
| | | Symbol;Acc:16639] |
| APTX | | aprataxin [Source:HGNC Symbol;Acc:15984] |
| ZFR | | zinc finger RNA binding protein [Source:HGNC |
| | | Symbol;Acc:17277] |
| UPF1 | | UPF1 regulator of nonsense transcripts homolog (yeast) |
| | | [Source:HGNC Symbol;Acc:9962] |
| KIN | | KIN, antigenic determinant of recA protein homolog |
| | | (mouse) [Source:HGNC Symbol;Acc:6327] |
| ZNF239 | | zinc finger protein 239 [Source:HGNC |
| | | Symbol;Acc:13031] |
| ZNF74 | | zinc finger protein 74 [Source:HGNC |
| | | Symbol;Acc:13144] |
| SNRPC | | small nuclear ribonucleoprotein polypeptide C |
| | | [Source:HGNC Symbol;Acc:11157] |
| C1orf25 | | TRIVI1-like protein [Source:UniProtKB/Swiss- |
| | | Prot;Acc:Q7Z2T5] |
| ZFR2 | | zinc finger RNA binding protein 2 [Source:HGNC |
| | | Symbol;Acc:29189] |
| IREB2 | ACO3; IRP2; | iron-responsive element binding protein 2 |
| | IRP2AD; | [Source:HGNC Symbol;Acc:6115] |
| | FLJ23381; IREB2 | |
| ACO1 | IRP1; ACONS; | aconitase 1, soluble [Source:HGNC Symbol;Acc:117] |
| | IREB1; IREBP; | |
| | IREBP1; ACO1 | |
| TROVE2 | RO60; SSA2; | TROVE domain family, member 2 [Source:HGNC |
| | RORNP; | Symbol;Acc:11313] |
| TEP1 | p240; TLP1; TP1; | telomerase-associated protein 1 [Source:HGNC |
| | TROVEl; | Symbol;Acc:11726] |
| | VAULT2 | |
| GAPDH | GAPD | glyceraldehyde-3-phosphate dehydrogenase |
| | | [Source:HGNC Symbol;Acc:4141] |
| ZRANB2 | | zinc finger, RAN-binding domain containing 2 |
| | | [Source:HGNC Symbol;Acc:13058] |

The RNA regulatory domain may be a protein selected from Table 1 or Table 2 or a functional domain from a protein selected from the list of proteins in Table 1 or 2. In some embodiments, the RNA regulatory domain comprises a fragment from a protein selected from the list of proteins in Table 1 or 2. In some embodiments, the RNA regulatory domain comprises a protein having at least, at most, or exactly 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, or 60% (or any derivable range therein) homology or sequence identity to a protein of Table 1 or 2 or a fragment of a protein from Table 1 or 2.

In some embodiments, the RNA regulatory domain comprises at least, at most, or exactly 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 contiguous amino acids (or any derivable range therein) of a protein from Table 1 or Table 2.

In some embodiments, the RNA regulatory domain comprises a fragment of a protein from Table 1 or 2, wherein the fragment has one or more of the following activities: methylation, 5′-3′ guanylylation, phosphoribosylation, deamination, carbamoylation, isopentenylation, agmatinylation, acetylation, lysylation, O/S exchange, galactosylation, glutamylation, mannosylation, hydrogenation, pseudouridine formation, carboxymethylaminomethylation, aminomethylation, decarboxymethylation, dehydrogenation, carboxymethylation, hydroxylation, methylthiolation, 3-amino-3-carboxypropylation, demethylation, 5′-5′ guanylylation, dephosphorylation, nuclease, editing, RNA transport, translational activation, translational repression, single-stranded RNA cleavage activity, double-stranded RNA cleavage activity, and RNA binding activity.

In some embodiments, the RNA regulatory domain is at or near the carboxy-terminus of the RNA hairpin binding protein. In some embodiments, the RNA regulatory domain is at or near the amino-terminus of the RNA hairpin binding protein. In some embodiments, the RNA regulatory domain is fused by way of a peptide bond to the RNA hairpin binding protein. In some embodiments, the RNA regulatory domain is linked to the RNA hairpin binding protein by a linker moiety.

### II. RNA HAIRPIN BINDING DOMAINS AND HAIRPIN STRUCTURES

Other RNA hairpin binding domains and hairpin structures that they bind are known in the art and can be used in the systems, compositions, fusion proteins, kits, vectors, and methods of the disclosure. For example, embodiments include a RNA hairpin binding domain and hairpin structure according to the following table (Table 3), which lists proteins comprising RNA hairpin binding domains and the hairpin structure that they specifically bind to:

TABLE 3
RNA hairpin binding domain

| RNA hairpin | |
|---|---|
| binding domain | Hairpin structure |
| MS2 coat protein | AAACAUGAGGAUUACCCAUGU (SEQ ID NO: 107) |
| (MCP) | AAACAUGAGGAUCACCCAUGU (SEQ ID NO: 108) |
| (bacteriophage) | |
| λ N | GCCCUGAAGAAGGGC (SEQ ID NO: 109); |
| (bacteriophage) | GCCCUGAAAAAGGGC (SEQ ID NO: 110) |
| PP7 | UAAGGAGUUUAUAUGGAAACCCUUA (SEQ ID NO: 111) |
| (Pseudomonas | |
| aeroginosa) | |
| Iron Responsive | CAGWGH wherein W is A or U and H is A, C, or U; |
| Protein (IRP) | NNNNNUGCNNNNNCAGUGNNNNNNCNNNNN, wherein n is any |
| | nucleotide |
| Qβ | AAACAUGAGGAUUACCCAUGU (SEQ ID NO: 107); |
| | AAACAUGAGGAUCACCCAUGU (SEQ ID NO: 108); |
| | AUGCAUGUCUAAGACAGCAU (SEQ ID NO: 114) |
| GA | AAACAUGAGGAUUACCCAUGU (SEQ ID NO: 107); |
| | AAACAUGAGGAUCACCCAUGU (SEQ ID NO: 108); |
| | AAAACAUAAGGAAAACCUAUGUU (SEQ ID NO: 115) |
| Bovine | GGCUCGUGUAGCUCAUUAGCUCCGAGCC (SEQ ID NO: 112) |
| immunodeficiency | |
| virus (BIV) | |
| transactivator of | |
| transcription (tat) | |
| U1A (HUMAN) | AUUGCAC; |
| | GGAAUCCAUUGCACUCCGGAUUUCACTAG (SEQ ID NO: 113); |
| | GGCCAGAUCUGAGCCUGGGAGCUCUCUGGCC (SEQ ID NO: 1) |
| SLBP (HUMAN) | CCAAAGGCUCUUCUCAGAGCCACCCA (SEQ ID NO: 2) |
| KU7 (HUMAN) | GGCGUCCCUCCCGAAGCUGCGCGCUCGGUCGAACAGGACG |
| | ACC (SEQ ID NO: 83) |
| NUCLEOLIN | UCCCGA (SEQ ID NO: 84); |
| (HUMAN) | GGCCGAAAUCCCGAAUGAGGCC (SEQ ID NO: 85); |
| | GGAUGCCUCCCGAGUGCAUCC (SEQ ID NO: 86). |

It is contemplated that multiple RNA hairpin binding domains and/or the RNA regulatory domain may be used in a multiplexed fashion by using RNA hairpin binding domains that bind to different hairpin structures to target multiple different RNAs in the same cell. The different RNAs may be modulated in the same or in different ways. For example, one RNA may be modulation with translational activation, while a second RNA may be modulated with translational repression in the same cell. Therefore, the systems of the disclosure can be used in a multiplexed fashion for the modulation of at least 2, 3, 4, 5, 6, 7, 8, 9, 10 or more RNAs in one cell, tissue, or organisms.

### III. NUCLEIC ACIDS

In certain embodiments, there are recombinant nucleic acids encoding the proteins, polypeptides, regulatory domains, or RNA targeting molecules described herein.

As used in this application, the term “polynucleotide” refers to a nucleic acid molecule that either is recombinant or has been isolated free of total genomic nucleic acid. Included within the term “polynucleotide” are oligonucleotides (nucleic acids 100 residues or fewer in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein (see above).

In particular embodiments, there are isolated nucleic acid segments and recombinant vectors incorporating nucleic acid sequences that encode a polypeptides (e.g., a polymerase, RNA polymerase, one or more truncated polymerase domains or interaction components that are polypeptides) that drive gene transcription dependent on polymerase activity from the polymerase domains when the interaction components interact. The term “recombinant” may be used in conjunction with a polypeptide or the name of a specific polypeptide, and this generally refers to a polypeptide produced from a nucleic acid molecule that has been manipulated in vitro or that is a replication product of such a molecule.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

A. Vectors

Polypeptides may be encoded by a nucleic acid molecule. The nucleic acid molecule can be in the form of a nucleic acid vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Vectors may be used in a host cell to produce a polymerase, RNA polymerase, one or more truncated polymerase domains or interaction components that are fused, attached or linked to the one or more truncated RNA polymerase domains.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

B. Cells

The disclosure provides methods for modifying a target RNA of interest, in particular in prokaryotic cells, eukaryotic cells, tissues, organs, or organisms, more in particular in mammalian cells, tissues, organs, or organisms. The target RNA may be comprised in a nucleic acid molecule within a cell. In some embodiments, the target RNA is in a eukaryotic cell, such as a mammalian cell or a plant cell. The mammalian cell many be a human, non-human primate, bovine, porcine, rodent or mouse cell. The cell may be a non-mammalian eukaryotic cell such as poultry, fish or shrimp. The plant cell may be of a crop plant such as cassava, corn, sorghum, wheat, or rice. The plant cell may also be of an algae, tree or vegetable. The modulation of the RNA induced in the cell by the methods, systems, and compositions of the disclosure may be such that the cell and progeny of the cell are altered for improved production of biologic products such as an antibody, starch, alcohol or other desired cellular output. The modulation of the RNA induced in the cell may be such that the cell and progeny of the cell include an alteration that changes the biologic product produced.

The mammalian cell may be a human or non-human mammal, e.g., primate, bovine, ovine, porcine, canine, rodent, Leporidae such as monkey, cow, sheep, pig, dog, rabbit, rat or mouse cell. The cell may be a non-mammalian eukaryotic cell such as poultry bird (e.g., chicken), vertebrate fish (e.g., salmon) or shellfish (e.g., oyster, clam, lobster, shrimp) cell. The cell may also be a plant cell. The plant cell may be of a monocot or dicot or of a crop or grain plant such as cassava, com, sorghum, soybean, wheat, oat or rice. The plant cell may also be of an algae, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.).

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

C. Expression Systems

Numerous expression systems exist that comprise at least a part or all of the compositions discussed above. Prokaryote- and/or eukaryote-based systems can be employed for use with an embodiment to produce nucleic acid sequences, or their cognate polypeptides, proteins and peptides. For example, the vectors, fusion proteins, RNA hairpin binding proteins, RNA targeting molecules, RNA regulatory domain, and accessory proteins of the disclosure may utilize an expression system, such as an inducible or constitutive expression system. Many such systems are commercially and widely available.

The insect cell/baculovirus system can produce a high level of protein expression of a heterologous nucleic acid segment, such as described in U.S. Patent Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems, other examples of expression systems include STRATAGENE®'s COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

D. Conjugation of Nucleic Acids and Polypeptides

Embodiments of the disclosure relate to the conjugation of nucleic acids to polypeptides. Methods of conjugation of nucleic acids to polypeptides are known in the art and include those described below. Embodiments of the disclosure relate to methods of making nucleic acid-polypeptide molecules and the molecules themselves wherein the nucleic acid has been conjugated to the polypeptide by way of a method described herein. One such example includes click chemistry. The “click reaction”, also known as “click chemistry” is a name often used to describe a stepwise variant of the Huisgen 1,3-dipolar cycloaddition of azides and alkynes to yield 1,2,3-triazole. This reaction is carried out under ambient conditions, or under mild microwave irradiation, typically in the presence of a Cu(I) catalyst, and with exclusive regioselectivity for the 1,4-disubstituted triazole product when mediated by catalytic amounts of Cu(I) salts [V. Rostovtsev, L. G. Green, V. V. Fokin, K. B. Sharpless, Angew. Chem. Int. Ed. 2002, 41, 2596; H. C. Kolb, M. Finn, K. B. Sharpless, Angew Chem., Int. Ed. 2001, 40, 2004].

In other conjugation methods, a mutant form of the human DNA repair protein O6-alkylguanine-DNA alkyltransferase reacts rapidly and specifically with O6-benzylguanin (BG) and also with derivatives that carry a large moiety linked to the benzyl group. With guanine as the leaving group, the benzyl moiety becomes covalently attached to a cysteine in the active site of the enzyme. The enzyme has also been mutagenized to become specific for O6-benzylcytosine (BC) in a similar manner. These enzyme domains (about 20 kDa) are commercially available as SNAP and CLIP tags, respectively.

A further conjugation method utilizes the Halo tag. The Halo tag makes use of a chemical reaction orthogonal to eukaryotes, i.e. the dehalogenation of haloalkane ligands, thus, leading to highly specific covalent labelling of the tag, and therefore protein, in both live and fixed cells.

E. Nucleic Acid Modifications

The oligonucleotides of the disclosure, such as the RNA targeting molecules and other nucleic acids described herein may have modifications that increase the stability of the nucleic acid. In some embodiments, the RNA targeting molecule is an oligonucleotide analogs. The term “oligonucleotide analog” refers to compounds which function like oligonucleotides but which have non-naturally occurring portions. Oligonucleotide analogs can have altered sugar moieties, altered base moieties or altered inter-sugar linkages. The term “oligomers” is intended to encompass oligonucleotides, oligonucleotide analogs or oligonucleosides. Thus, in speaking of “oligomers” reference is made to a series of nucleosides or nucleoside analogs that are joined via either natural phosphodiester bonds or other linkages, including the four atom linkers. Although the linkage generally is from the 3′ carbon of one nucleoside to the 5′ carbon of a second nucleoside, the term “oligomer” can also include other linkages such as 2′-5′ linkages.

Oligonucleotide analogs also can include other modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true nucleic acid species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

The present disclosure concerns modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound. Preferred linkages have structure CH2 —RA —NR1CH2, CH2 —NR1—RA —CH2, RA —NR1—CH2 —CH2, CH2 —CH2 —NR1 —RA, CH2 —CH2 —RA —NR1, or NR1 —RA —CH2 —CH2 where RA is O or NR2.

Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon is modified at its terminal 3′ site, while a “downstream” synthon is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass [53], TentaGel Support—an aminopolyethyleneglycol derivatized support [54] or Poros—a copolymer of polystyrene/divinylbenzene. Attachment and cleavage of nucleotides and oligonucleotides can be effected via standard procedures [55]. As used herein, the term solid support further includes any linkers (e.g., long chain alkyl amines and succinyl residues) used to bind a growing oligonucleoside to a stationary phase such as CPG.

1. Locked Nucleotides

In some embodiments, the nucleic acid of the disclosure, such as the RNA targeting molecule comprises a locked nucleic acid. A locked nucleic acid (LNA or Ln), also referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. Such oligomers are synthesized chemically and are commercially available. The locked ribose conformation enhances base stacking and backbone pre-organization. This significantly increases the hybridization properties (melting temperature) of oligonucleotides.

2. Ethylene Bridged Nucleotides

In some embodiments, the nucleic acid of the disclosure, such as the RNA targeting molecule comprises one or more ethylene bridged nucleotides. Ethylene-bridged nucleic acids

(ENA or En) are modified nucleotides with a 2′-O, 4′C ethylene linkage. Like locked nucleotides, these nucleotides also restrict the sugar puckering to the N-conformation of RNA.

3. Peptide Nucleic Acids

In some embodiments, the nucleic acid of the disclosure, such as the RNA targeting molecule comprises one or more peptide nucleic acids. Peptide nucleic acids (PNA or Pn) mimic the behavior of DNA and binds complementary nucleic acid strands. The term, “peptide,” when used herein may also refer to a peptide nucleic acid. PNA is an artificially synthesized polymer similar to DNA or RNA. DNA and RNA have a deoxyribose and ribose sugar backbone, respectively, whereas PNA's backbone is composed of repeating N-(2-aminoethyl)-glycine units linked by peptide bonds. The various purine and pyrimidine bases are linked to the backbone by a methylene bridge (—CH2-) and a carbonyl group (—(C═O)—). PNAs are depicted like peptides, with the N-terminus at the first (left) position and the C-terminus at the last (right) position.

Since the backbone of PNAs contains no charged phosphate groups, the binding between PNA/DNA strands is stronger than between DNA/DNA strands due to the lack of electrostatic repulsion. PNAs are not easily recognized by either nucleases or proteases, making them resistant to degradation by enzymes. PNAs are also stable over a wide pH range. In some aspects, the PNAs described herein have improved cytosolic delivery over other PNAs.

4. 5′(E)-Vinyl-Phosphonate (VP) Modification

In some embodiments, the nucleic acid of the disclosure, such as the RNA targeting molecule comprises one or more 5′(E)-vinyl-phosphonate (VP) modifications. 5′-Vinyl-phosphonate modifications (metabolically stable phosphate mimics) have been reported to enhance the metabolic stability and the potency of oligonucleotides.

5. Morpholinos

In some embodiments, the nucleic acid of the disclosure, such as the RNA targeting molecule comprises a morpholino. Morpholinos are synthetic molecules that are the product of a redesign of natural nucleic acid structure. Usually 25 bases in length, they bind to complementary sequences of RNA or single-stranded DNA by standard nucleic acid base-pairing. In terms of structure, the difference between Morpholinos and DNA is that, while Morpholinos have standard nucleic acid bases, those bases are bound to methylenemorpholine rings linked through phosphorodiamidate groups instead of phosphates. The figure compares the structures of the two strands depicted there, one of RNA and the other of a Morpholino. Replacement of anionic phosphates with the uncharged phosphorodiamidate groups eliminates ionization in the usual physiological pH range, so Morpholinos in organisms or cells are uncharged molecules. The entire backbone of a Morpholino is made from these modified subunits.

### IV. DELIVERY VEHICLES

The current disclosure contemplates several delivery systems compatible with nucleic acids that provide for roughly uniform distribution and have controllable rates of release. A variety of different media are described below that are useful in creating nucleic acid delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated by this disclosure comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcinol-aldehyde type bioadhesives, polyacrylic acid and copolymers and block copolymers thereof.

A. Microparticles

Some embodiments of the present disclosure contemplate a delivery system comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, pseudo-poly(amino acids), polyhydroxybutyrate-related copolymers, polyanhydrides, polymethylmethacrylate, poly(ethylene oxide), lecithin and phospholipids.

B. Liposomes

One embodiment of the disclosure contemplates liposomes capable of attaching and releasing nucleic acids conjugates, polypeptides, and fusion proteins as described herein. Liposomes are microscopic spherical lipid bilayers surrounding an aqueous core that are made from amphiphilic molecules such as phospholipids. For example, a liposome may trap a nucleic acid between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phospholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes allow the simultaneous storage of normally immiscible compounds and the capability of their controlled release.

In some embodiments, the disclosure contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present disclosure contemplates a delivery system comprising liposomes that provides controlled release of at least one molecule described herein. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

C. Microspheres, Microparticles and Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease™, Alkerme's: Cambridge, Mass.). For example, a freeze dried medium comprising at least one therapeutic agent is homogenized in a suitable solvent and sprayed to manufacture microspheres in the range of 20 to 90 μm Techniques are then followed that maintain sustained release integrity during phases of purification, encapsulation and storage. Scott et al., Improving Protein Therapeutics With Sustained Release Formulations, Nature Biotechnology, Volume 16:153-157 (1998). Modification of the microsphere composition by the use of biodegradable polymers can provide an ability to control the rate of nucleic acid release. Miller et al., Degradation Rates of Oral Resorbable Implants {Polylactates and Polyglycolates: Rate Modification and Changes in PLA/PGA Copolymer Ratios, J. Biomed. Mater. Res., Vol. 11:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a nucleic acid is added to the biodegradable polymer metal salt solution. The weight ratio of a nucleic acid to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and nucleic acid is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and nucleic acid mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a nucleic acid for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere™ (Macromed) is a controlled release microsphere system. These particular microsphere's are available in uniform sizes ranging between 5-500 μm and composed of biocompatible and biodegradable polymers. Specific polymer compositions of a microsphere can control the nucleic acid release rate such that custom-designed microspheres are possible, including effective management of the burst effect. ProMaxx™ (Epic Therapeutics, Inc.) is a protein-matrix delivery system. The system is aqueous in nature and is adaptable to standard pharmaceutical delivery models. In particular, ProMaxx™ are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated nucleic acid, therefore, will be selected for the pH environment in which dissolution is desired and stored in a pH preselected to maintain stability. Examples of pH sensitive material useful as encapsulants are Eudragit™ L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a nucleic acid is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

In one embodiment, the present invention contemplates microparticles formed by spray-drying a composition comprising fibrinogen or thrombin with a nucleic acid. Preferably, these microparticles are soluble and the selected protein (i.e., fibrinogen or thrombin) creates the walls of the microparticles. Consequently, the nucleic acids are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well known material.

### V. PROTEINACEOUS COMPOSITIONS

The polypeptides or polynucleotides of the disclosure, such as the CIRT fusion proteins, stabilizer polypeptide, linker, RNA hairpin binding domain, NES, RNA regulatory domain, tag, NLS, RNA targeting molecule, hairpin region of the RNA targeting molecule, helical region, or targeting region of the RNA targeting molecule may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOs:1-132.

The polypeptides or polynucleotides of the disclosure, such as the CIRT fusion proteins, stabilizer polypeptide, linker, RNA hairpin binding domain, NES, RNA regulatory domain, tag, NLS, RNA targeting molecule, hairpin region of the RNA targeting molecule, helical region, or targeting region of the RNA targeting molecule may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of SEQ ID NO:1-132.

In some embodiments, the fusion protein may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) of SEQ ID NOs: 87-106 or 128-132.

In some embodiments, the fusion protein may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOs: 87-106 or 128-132.

In some embodiments, the fusion protein may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOs:87-106 or 128-132 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NOS:87-106 or 128-132.

In other embodiments, the stabilizer polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90, or any derivable range therein of SEQ ID NO:5, 19, or 20.

In some embodiments, the stabilizer polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 (or any derivable range therein) contiguous amino acids of SEQ ID NO:5, 19, or 20.

In some embodiments, the stabilizer polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 (or any derivable range therein) contiguous amino acids of SEQ ID NO:5, 19, or 20 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NO:5, 19, or 20.

In some embodiments, the RNA hairpin binding domain may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, or 102, (or any derivable range therein) of SEQ ID NOs:7 or 18.

In some embodiments, the RNA hairpin binding domain may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, or 102 (or any derivable range therein) contiguous amino acids of SEQ ID NOs:7 or 18.

In some embodiments, the RNA hairpin binding domain may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, or 102 (or any derivable range therein) contiguous amino acids of SEQ ID NOs:7 or 18 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NOS:7 or 18.

In some embodiments, the RNA regulatory domain may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, or 364 (or any derivable range therein) of SEQ ID NOs:9, 11, 15-17, or 123-125.

In some embodiments, the RNA regulatory domain may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, or 364 (or any derivable range therein) contiguous amino acids of SEQ ID NOs:9, 11, 15-17, or 123-125.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501,

502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 of any of SEQ ID NOS:1-132 and comprising 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 contiguous nucleotides or polypeptides of any of SEQ ID NOS:1-132.

In some embodiments, the RNA regulatory domain may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, or 364 (or any derivable range therein) contiguous amino acids of SEQ ID NOs:9, 11, 15-17, or 123-125 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NOs:9, 11, 15-17, or 123-125.

In some embodiments, the hairpin structure, such as the stem, loop, or both stem and loop, of the RNA targeting molecule may comprise nucleic acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43 (or any derivable range therein) of SEQ ID NOs:1, 2, 26, 28, or 83-86.

In some embodiments, the hairpin structure, such as the stem, loop, or both stem and loop, of the RNA targeting molecule may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43 (or any derivable range therein) contiguous nucleic acids of SEQ ID NOs:1, 2, 26, 28, or 83-86.

In some embodiments, the hairpin structure, such as the stem, loop, or both stem and loop, of the RNA targeting molecule may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43 (or any derivable range therein) contiguous nucleic acids of SEQ ID NOs:1, 2, 26, 28, or 83-86 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar or identical with one of SEQ ID NOs:1, 2, 26, 28, or 83-86.

In some embodiments, the RNA targeting region or the RNA targeting molecule may comprise nucleic acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 (or any derivable range therein) of SEQ ID NOs:30-62.

In some embodiments, the RNA targeting region or the RNA targeting molecule may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 (or any derivable range therein) contiguous nucleic acids of SEQ ID NOs:30-62.

In some embodiments, the RNA targeting region or the RNA targeting molecule may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 (or any derivable range therein) contiguous nucleic acids of SEQ ID NOs:30-62 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar or identical with one of SEQ ID NOs:30-62.

In some aspects, the stabilizer polypeptide may have one or more substitutions that reduce or eliminate binding to endogenous proteins. In some aspects, the stabilizer polypeptide may have one or more substitutions that reduce or eliminate an activity directed to an endogenous protein.

The polypeptides and nucleic acids of the disclosure, such as the CIRT fusion proteins, stabilizer polypeptide, linker, RNA hairpin binding domain, NES, RNA regulatory domain, tag, NLS, RNA targeting molecule, hairpin region of the RNA targeting molecule, helical region, or targeting region of the RNA targeting molecule may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 595, 596, 597, 598, 599, 600, 601, 602, 603, 604, 605, 606, 607, 608, 609, 610, 611, 612, 613, 614, or 615 substitutions.

The substitution may be at amino acid position or nucleic acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, or 615 of one of SEQ ID NO:1-132.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other embodiments, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4, 554, 101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific embodiments, all or part of proteins described herein can also be synthesized in solution or on a solid support in accordance with conventional techniques. Various automatic synthesizers are commercially available and can be used in accordance with known protocols. See, for example, Stewart and Young, (1984); Tam etal., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### VI. SEQUENCES

TAR hairpin Scaffold:
(SEQ ID NO: 1)
ggccagaucugagccugggagcucucuggcc
Human SLBP hairpin scaffold:
(SEQ ID NO: 2)
ccaaaggcucuucucagagccaccca

| Name: | Description: | |
|---|---|---|
| MBP- | MBP | MKIEEGKLVIWINGDKGYNGLAEVGKKFEKDTGIKVTVEHP |
| CIRTS-1 | signaling | DKLEEKFPQVAATGDGPDBFWAHDRFGGYAQSGLLAEITP |
| | peptide | DKAFQDKLYPFTWDAVRYNGKLIAYPIAVEALSLIYNKDLL |
| | | PNPPKTWEEIPALDKELKAKGKSALMFNLQEPYFTWPLIAA |
| | | DGGYAFKYENGKYDIKDVGVDNAGAKAGLTFLVDLIKNKH |
| | | MNADTDYSIAEAAFNKGETAMTINGPWAWSNIDTSKVNYG |
| | | VTVLPTFKGQPSKPFVGVLSAGINAASPNKELAKEFLENYLL |
| | | TDEGLEAVNKDKPLGAVALKSYEEELAKDPRIAATMENAQ |
| | | KGEIMPNIPQMSAFWYAVRTAVINAASGRQTVDEALKDAQ |
| | | TNS (SEQ ID NO: 3) |
| | | ITSLYKKAGSETVRFQSHHHHHHSSGVDLGTENLYFQSNA |
| | | (SEQ ID NO: 4) |
| | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSGGSGGSGGSGGSGGS (SEQ ID NO: 6) |
| | dead Pin | MELEIRPLFLVPDTNGFIDHLASLARLLESRKYILVVPLIVINE |
| | domain | LDGLAKGQETDHRAGGYARVVQEKARKSIEFLEQRFESRDS |
| | | CLRALTSRGNELESIAFRSEDITGQLGNNDDLILSCCLHYCK |
| | | DKAKDFMPASKEEPIRLLREVVLLTDDRNLRVKALTRNVPV |
| | | RDIPAFLTWAQVG (SEQ ID NO: 9) |
| | | SSG |
| | 6H tag | HEIHHHE (SEQ ID NO: 10) |
| CIRTS-0 | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSGGSGGSGGSGGSGGS (SEQ ID NO: 6) |
| | dead Pin | MELEIRPLFLVPDTNGFIDHLASLARLLESRKYILVVPLIVINE |
| | domain | LDGLAKGQETDHRAGGYARVVQEKARKSIEFLEQRFESRDS |
| | | CLRALTSRGNELESIAFRSEDITGQLGNNADLILSCCLHYCK |
| | | DKAKDFMPASKEEPIRLLREVVLLTDDRNLRVKALTRNVPV |
| | | RDIPAFLTWAQVG (SEQ ID NO: 11) |
| | | GS |
| | 3xFLAG | DYKDHDGDYKDHDIDYKDDDDK (SEQ ID NO: 12) |
| CIRTS-1 | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | linker | GGSGGSGGSGGSGGSGGS (SEQ ID NO: 6) |
| | Pin domain | MELEIRPLFLVPDTNGFIDHLASLARLLESRKYILVVPLIVINE |
| | | LDGLAKGQETDHRAGGYARVVQEKARKSIEFLEQRFESRDS |
| | | CLRALTSRGNELESIAFRSEDITGQLGNNDDLILSCCLHYCK |
| | | DKAKDFMPASKEEPIRLLREVVLLTDDRNLRVKALTRNVPV |
| | | RDIPAFLTWAQVG (SEQ ID NO: 9) |
| | NLS | KRPAATKKAGQAKKKK (SEQ ID NO: 13) |
| | | GS |
| | Tag | DYKDDDDK (SEQ ID NO: 14) |
| CIRTS-2 | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSGGSGGSGGSGGSGGS (SEQ ID NO: 6) |
| | N terminal | MSATSVDTQRTKGQDNKVQNGSLHQKDTVHDNDFEPYLT |
| | YTHDF1 | GQSNQSNSYPSMSDPYLSSYYPPSIGFPYSLNEAPWSTAGDP |
| | | PIPYLTTYGQLSNGDHHFMHDAVFGQPGGLGNNIYQHRFNF |
| | | FPENPAFSAWGTSGSQGQQTQSSAYGSSYTYPPSSLGGTVV |
| | | DGQPGFHSDTLSKAPGMNSLEQGMVGLKIGDVSSSAVKTV |
| | | GSVVSSVALTGVLSGNGGTNVNMPVSKPTSWAAIASKPAK |
| | | PQPKMKTKSGPVMGGGLPPPPIKHNMDIGTWDNKGPVPKA |
| | | PVPQQAPSPQAAPQPQQVAQPLPAQPPALAQPQYQSPQQPP |
| | | QTRWVAPRNRNAAFGQSGGAGSDSNSPGNVQPNSAPSVES |
| | | (SEQ ID NO: 15) |
| | | GS |
| | Tag | DYKDDDDK (SEQ ID NO: 14) |
| CIRTS-3ₜ | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSGGSGGSGGSGGSGGS (SEQ ID NO: 6) |
| | YTHDF2 | DAMFGQPGALGSTPFLGQHGFNFFPSGIDFSAWGNNSSQGQ |
| | (101-200) | STQSSGYSSNYAYAPSSLGGAMIDGQSAFANETLNKAPGMN |
| | | TIDQGMAA (SEQ ID NO: 16) |
| | | GS |
| | Tag | DYKDDDDK (SEQ ID NO: 14) |
| CIRTS-3 | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSGGSGGSGGSGGSGGS (SEQ ID NO: 6) |
| | N-terminal | MSASSLLEQRPKGQGNKVQNGSVHQKDGLNDDDFEPYLSP |
| | YTHDF2 | QARPNNAYTAMSDSYLPSYYSPSIGFSYSLGEAAWSTGGDT |
| | (1-200) | AMPYLTSYGQLSNGEPHFLPDAMFGQPGALGSTPFLGQHGF |
| | | NFFPSGIDFSAWGNNSSQGQSTQSSGYSSNYAYAPSSLGGA |
| | | MIDGQSAFANETLNKAPGMNTIDQGMAA(SEQIDNO:17) |
| | | GS |
| | tag | DYKDDDDK (SEQ ID NO: 14) |
| CIRTS-4ₜ | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | SLBP 124- | ADFETDESVLMRRQKQINYGKNTIAYDRYIKEVPRHLRQPGI |
| | 198 | HPKTPNKFKKYSRRSWDQQIKLWKVALHFWD (SEQ ID |
| | | NO: 18) |
| | HIV NES | LQLPPLERLTL (SEQ ID NO: 8) |
| | linker | GGSGGSGGSGGSGGSGGS (SEQ ID NO: 6) |
| | YTHDF2 | DAMFGQPGALGSTPFLGQHGFNFFPSGIDFSAWGNNSSQGQ |
| | (101-200) | STQSSGYSSNYAYAPSSLGGAMIDGQSAFANETLNKAPGMN |
| | | TIDQGMAA (SEQ ID NO: 16) |
| | | GS |
| | tag | DYKDDDDK (SEQ ID NO: 14) |
| CIRTS-4 | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | SLBP 124- | ADFETDESVLMRRQKQINYGKNTIAYDRYIKEVPRHLRQPGI |
| | 198 | HPKTPNKFKKYSRRSWDQQIKLWKVALHFWD (SEQ ID |
| | | NO: 18) |
| | HIV NES | LQLPPLERLTL (SEQ ID NO: 8) |
| | linker | GGSGGSGGSGGSGGSGGS (SEQ ID NO: 6) |
| | YTHDF2 | MSASSLLEQRPKGQGNKVQNGSVHQKDGLNDDDFEPYLSP |
| | (1-200) | QARPNNAYTAMSDSYLPSYYSPSIGFSYSLGEAAWSTGGDT |
| | | AMPYLTSYGQLSNGEPHFLPDAMFGQPGALGSTPFLGQHGF |
| | | NFFPSGIDFSAWGNNSSQGQSTQSSGYSSNYAYAPSSLGGA |
| | | MIDGQSAFANETLNKAPGMNTIDQGMAA (SEQ ID NO: 17) |
| | | GS |
| | tag | DYKDDDDK (SEQ ID NO: 14) |
| CIRTS-5ₜ | HBEGF | MRVTLSSKPQALATPNKEEHGKRKKKGKGLGKKRDPCLRK |
| | | YKDFCIHGECKYVKELRAPSCICHPGYHGERCHGLS (SEQ ID |
| | | NO: 19) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSGGSGGSGGSGGSGGS (SEQ ID NO: 6) |
| | YTHDF2 | DAMFGQPGALGSTPFLGQHGFNFFPSGIDFSAWGNNSSQGQ |
| | (101-200) | STQSSGYSSNYAYAPSSLGGAMIDGQSAFANETLNKAPGMN |
| | | TIDQGMAA (SEQ ID NO: 16) |
| | | GS |
| | tag | DYKDDDDK (SEQ ID NO: 14) |
| CIRTS-5 | HBEGF | MRVTLSSKPQALATPNKEEHGKRKKKGKGLGKKRDPCLRK |
| | | YKDFCIHGECKYVKELRAPSCICHPGYHGERCHGLS (SEQ ID |
| | | NO: 19) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSGGSGGSGGSGGSGGS (SEQ ID NO: 6) |
| | YTHDF2 | MSASSLLEQRPKGQGNKVQNGSVHQKDGLNDDDFEPYLSP |
| | (1-200) | QARPNNAYTAMSDSYLPSYYSPSIGFSYSLGEAAWSTGGDT |
| | | AMPYLTSYGQLSNGEPHFLPDAMFGQPGALGSTPFLGQHGF |
| | | NFFPSGIDFSAWGNNSSQGQSTQSSGYSSNYAYAPSSLGGA |
| | | MIDGQSAFANETLNKAPGMNTIDQGMAA (SEQ ID NO: 17) |
| | | GS |
| | tag | DYKDDDDK (SEQ ID NO: 14) |
| CIRTS-6ₜ | β-defensin | MGIINTLQKYYCRVRGGRCAVLSCLPKEEQIGKCSTRGRKC |
| | | CRRKK (SEQ ID NO: 20) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSGGSGGSGGSGGSGGS (SEQ ID NO: 6) |
| | YTHDF2 | DAMFGQPGALGSTPFLGQHGFNFFPSGIDFSAWGNNSSQGQ |
| | (101-200) | STQSSGYSSNYAYAPSSLGGAMIDGQSAFANETLNKAPGMN |
| | | TIDQGMAA (SEQ ID NO: 16) |
| | | GS |
| | tag | DYKDDDDK (SEQ ID NO: 14) |
| CIRTS-6 | β-defensin | MGIINTLQKYYCRVRGGRCAVLSCLPKEEQIGKCSTRGRKC |
| | | CRRKK (SEQ ID NO: 20) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSGGSGGSGGSGGSGGS (SEQ ID NO: 6) |
| | YTHDF2 | MSASSLLEQRPKGQGNKVQNGSVHQKDGLNDDDFEPYLSP |
| | (1-200) | QARPNNAYTAMSDSYLPSYYSPSIGFSYSLGEAAWSTGGDT |
| | | AMPYLTSYGQLSNGEPHFLPDAMFGQPGALGSTPFLGQHGF |
| | | NFFPSGIDFSAWGNNSSQGQSTQSSGYSSNYAYAPSSLGGA |
| | | MIDGQSAFANETLNKAPGMNTIDQGMAA (SEQ ID NO: 17) |
| | | GS |
| | tag | DYKDDDDK (SEQ ID NO: 14) |
| CIRTS-7 | β-defensin | MGIINTLQKYYCRVRGGRCAVLSCLPKEEQIGKCSTRGRKC |
| | | CRRKK (SEQ ID NO: 20) |
| | linker | GGSGGSGGSGGSGGSGGS (SEQ ID NO: 6) |
| | SLBP 124- | ADFETDESVLMRRQKQINYGKNTIAYDRYIKEVPRHLRQPGI |
| | 198 | HPKTPNKFKKYSRRSWDQQIKLWKVALHFWD (SEQ ID |
| | | NO: 18) |
| | HIV NES | LQLPPLERLTL (SEQ ID NO: 8) |
| | linker | GGSGGSGGSGGSGGSGGS (SEQ ID NO: 6) |
| | YTHDF2 | MSASSLLEQRPKGQGNKVQNGSVHQKDGLNDDDFEPYLSP |
| | (1-200) | QARPNNAYTAMSDSYLPSYYSPSIGFSYSLGEAAWSTGGDT |
| | | AMPYLTSYGQLSNGEPHFLPDAMFGQPGALGSTPFLGQHGF |
| | | NFFPSGIDFSAWGNNSSQGQSTQSSGYSSNYAYAPSSLGGA |
| | | MIDGQSAFANETLNKAPGMNTIDQGMAA (SEQ ID NO: 17) |
| | | GS |
| | tag | DYKDDDDK (SEQ ID NO: 14) |
| CIRTS-8 | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSGGSGGSGGSGGSGGS (SEQ ID NO: 6) |
| | Pin domain | MELEIRPLFLVPDTNGFIDHLASLARLLESRKYILVVPLIVINE |
| | | LDGLAKGQETDHRAGGYARVVQEKARKSIEFLEQRFESRDS |
| | | CLRALTSRGNELESIAFRSEDITGQLGNNDDLILSCCLHYCK |
| | | DKAKDFMPASKEEPIRLLREVVLLTDDRNLRVKALTRNVPV |
| | | RDIPAFLTWAQVG (SEQ ID NO: 9) |
| | | GS |
| | tag | DYKDDDDK (SEQ ID NO: 14) |
| CIRTS-9 | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSGGSGGSGGSGGSGGS (SEQ ID NO: 6) |
| | Pin domain | MELEIRPLFLVPDTNGFIDHLASLARLLESRKYILVVPLIVINE |
| | | LDGLAKGQETDHRAGGYARVVQEKARKSIEFLEQRFESRDS |
| | | CLRALTSRGNELESIAFRSEDITGQLGNNDDLILSCCLHYCK |
| | | DKAKDFMPASKEEPIRLLREVVLLTDDRNLRVKALTRNVPV |
| | | RDIPAFLTWAQVG (SEQ ID NO: 9) |
| | | GS |
| | tag | DYKDDDDK (SEQ ID NO: 14) |
| CIRTS-10 | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSGGSGGS (SEQ ID NO: 21) |
| | Pin domain | MELEIRPLFLVPDTNGFIDHLASLARLLESRKYILVVPLIVINE |
| | | LDGLAKGQETDHRAGGYARVVQEKARKSIEFLEQRFESRDS |
| | | CLRALTSRGNELESIAFRSEDITGQLGNNDDLILSCCLHYCK |
| | | DKAKDFMPASKEEPIRLLREVVLLTDDRNLRVKALTRNVPV |
| | | RDIPAFLTWAQVG (SEQ ID NO: 9) |
| | | GS |
| | tag | DYKDDDDK (SEQ ID NO: 14) |
| CIRTS-11 | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | L8 linker | SGSETPGTSESATPES (SEQ ID NO: 22) |
| | Pin domain | MELEIRPLFLVPDTNGFIDHLASLARLLESRKYILVVPLIVINE |
| | | LDGLAKGQETDHRAGGYARVVQEKARKSIEFLEQRFESRDS |
| | | CLRALTSRGNELESIAFRSEDITGQLGNNDDLILSCCLHYCK |
| | | DKAKDFMPASKEEPIRLLREVVLLTDDRNLRVKALTRNVPV |
| | | RDIPAFLTWAQVG (SEQ ID NO: 9) |
| | | GS |
| | tag | DYKDDDDK (SEQ ID NO: 14) |
| CIRTS-12 | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSG (SEQ ID NO: 23) |
| | ER/K 10 nm | EEEEKKKQQEEEAERLRRIQEEMEKERKRREEDEQRRRKEE |
| | | EERRMKLEMEAKRKQEEEERKKREDDEKRKKK (SEQ ID |
| | | NO: 24) |
| | linker | GSGGS (SEQ ID NO: 25) |
| | Pin domain | MELEIRPLFLVPDTNGFIDHLASLARLLESRKYILVVPLIVINE |
| | | LDGLAKGQETDHRAGGYARVVQEKARKSIEFLEQRFESRDS |
| | | CLRALTSRGNELESIAFRSEDITGQLGNNDDLILSCCLHYCK |
| | | DKAKDFMPASKEEPIRLLREVVLLTDDRNLRVKALTRNVPV |
| | | RDIPAFLTWAQVG (SEQ ID NO: 9) |
| | | GS |
| | tag | DYKDDDDK (SEQ ID NO: 14) |
| CIRTS-13 | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | L8 linker | SGSETPGTSESATPES (SEQ ID NO: 22) |
| | Y2 | DAMFGQPGALGSTPFLGQHGFNFFPSGIDFSAWGNNSSQGQ |
| | (101-200) | STQSSGYSSNYAYAPSSLGGAMIDGQSAFANETLNKAPGMN |
| | | TIDQGMAA (SEQ ID NO: 16) |
| | | GS |
| | tag | DYKDDDDK (SEQ ID NO: 14) |
| CIRTS-14 | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | linker | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | | GS |
| | HIV NES | LQLPPLERLTL (SEQ ID NO: 8) |
| | linker | GGSG (SEQ ID NO: 23) |
| | ER/K 10 nm | EEEEKKKQQEEEAERLRRIQEEMEKERKRREEDEQRRRKEE |
| | | EERRMKLEMEAKRKQEEEERKKREDDEKRKKK (SEQ ID |
| | | NO: 24) |
| | linker | GSGGS (SEQ ID NO: 25) |
| | Y2 | DAMFGQPGALGSTPFLGQHGFNFFPSGIDFSAWGNNSSQGQ |
| | (101-200) | STQSSGYSSNYAYAPSSLGGAMIDGQSAFANETLNKAPGMN |
| | | TIDQGMAA (SEQ ID NO: 16) |
| | | GS |
| | tag | DYKDDDDK (SEQ ID NO: 14) |
| CIRTS-7X | β-defensin 3 | MGIINTLQKYYCRVRGGRCAVLSCLPKEEQIGKCSTRGRKC |
| | | CRRKK (SEQ ID NO: 20) |
| | GGS₆ | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | GGS₆ | GGSGGSGGSGGSGGSGGS (SEQ ID NO: 6) |
| | hADAR | LHLDQTPSRQPIPSEGLQLHLPQVLADAVSRLVLGKFGDLTD |
| | (299-701) | NFSSPHARRKVLAGVVMTTGTDVKDAKVISVSTGTKCINGE |
| | | YMSDRGLALNDCHAEIISRRSLLRFLYTQLELYLNNKDDQK |
| | | RSIFQKSERGGFRLKENVQFHLYISTSPCGDARIFSPHEPILEE |
| | | PADRHPNRKARGQLRTKIESGEGTIPVRSNASIQTWDGVLQ |
| | | GERLLTMSCSDKIARWNVVGIQGSLLSIFVEPIYFSSIILGSLY |
| | | HGDHLSRAMYQRISNIEDLPPLYTLNKPLLSGISNAEARQPG |
| | | KAPNFSVNWTVGDSAIEVINATTGKDELGRASRLCKHALYC |
| | | RWMRVHGKVPSHLLRSKITKPNVYHESKLAAKEYQAAKAR |
| | | LFTAFIKAGLGAWVEKPTEQDQFSLTP (SEQ ID NO: 123) |
| | NLS | KRPAATKKAGQAKKKK (SEQ ID NO: 13) |
| | | GS |
| | Flag tag | DYKDDDDK (SEQ ID NO: 14) |
| CIRTS-8X | β-defensin 3 | MGIINTLQKYYCRVRGGRCAVLSCLPKEEQIGKCSTRGRKC |
| | | CRRKK (SEQ ID NO: 20) |
| | GGS₆ | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | GGS₆ | GGSGGSGGSGGSGGSGGS (SEQ ID NO: 6) |
| | hADAR | LHLDQTPSRQPIPSEGLQLHLPQVLADAVSRLVLGKFGDLTD |
| | (299-701) | NFSSPHARRKVLAGVVMTTGTDVKDAKVISVSTGTKCINGE |
| | E488Q | YMSDRGLALNDCHAEIISRRSLLRFLYTQLELYLNNKDDQK |
| | | RSIFQKSERGGFRLKENVQFHLYISTSPCGDARIFSPHEPILEE |
| | | PADRHPNRKARGQLRTKIESGQGTIPVRSNASIQTWDGVLQ |
| | | GERLLTMSCSDKIARWNVVGIQGSLLSIFVEPIYFSSIILGSLY |
| | | HGDHLSRAMYQRISNIEDLPPLYTLNKPLLSGISNAEARQPG |
| | | KAPNFSVNWTVGDSAIEVINATTGKDELGRASRLCKHALYC |
| | | RWMRVHGKVPSHLLRSKITKPNVYHESKLAAKEYQAAKAR |
| | | LFTAFIKAGLGAWVEKPTEQDQFSLTP (SEQ ID NO: 124) |
| | NLS | KRPAATKKAGQAKKKK (SEQ ID NO: 13) |
| | | GS |
| | Flag tag | DYKDDDDK (SEQ ID NO: 14) |
| CIRTS-9X | β-defensin 3 | MGIINTLQKYYCRVRGGRCAVLSCLPKEEQIGKCSTRGRKC |
| | | CRRKK (SEQ ID NO: 20) |
| | GGS₆ | GGSGGSGGSGGSGGSGGS (SEQ ID NO: 6) |
| | TBP6.7 | MAVPETRPNHTIYINNLNSKIKKDELKKSLYAIFSQFGQILDI |
| | | LVPRQRTPRGQAFVIFKEVSSATNALRSMQGFPFYDKPMRI |
| | | QYAKTDKRIPAKMKGTFV (SEQ ID NO: 7) |
| | NES | GSLQLPPLERLTL (SEQ ID NO: 124) |
| | GGS₆ | GGSGGSGGSGGSGGSGGS (SEQ ID NO: 6) |
| | NYTHDF1 | MSATSVDTQRTKGQDNKVQNGSLHQKDTVHDNDFEPYLT |
| | | GQSNQSNSYPSMSDPYLSSYYPPSIGFPYSLNEAPWSTAGDP |
| | | PIPYLTTYGQLSNGDHHFMHDAVFGQPGGLGNNIYQHRFNF |
| | | FPENPAFSAWGTSGSQGQQTQSSAYGSSYTYPPSSLGGTVV |
| | | DGQPGFHSDTLSKAPGMNSLEQGMVGLKIGDVSSSAVKTV |
| | | GSVVSSVALTGVLSGNGGTNVNMPVSKPTSWAAIASKPAK |
| | | PQPKMKTKSGPVMGGGLPPPPIKHNMDIGTWDNKGPVPKA |
| | | PVPQQAPSPQAAPQPQQVAQPLPAQPPALAQPQYQSPQQPP |
| | | QTRWVAPRNRNAAFGQSGGAGSDSNSPGNVQPNSAPSVES |
| | | (SEQ ID NO: 15) |
| | | GS |
| | Flag tag | DYKDDDDK (SEQ ID NO: 14) |
| CIRTS-10X | β-defensin 3 | MGIINTLQKYYCRVRGGRCAVLSCLPKEEQIGKCSTRGRKC |
| | | CRRKK (SEQ ID NO: 20) |
| | GGS₆ | GGSGGSGGSGGSGGSGGS (SEQ ID NO: 6) |
| | SLBP | ADFETDESVLMRRQKQINYGKNTIAYDRYIKEVPRHLRQPGI |
| | | HPKTPNKFKKYSRRSWDQQIKLWKVALHFWD (SEQ ID |
| | | NO: 18) |
| | NES | LQLPPLERLTL (SEQ ID NO: 8) |
| | GGS₆ | GGSGGSGGSGGSGGSGGS (SEQ ID NO: 6) |
| | YTHDF2 | MSASSLLEQRPKGQGNKVQNGSVHQKDGLNDDDFEPYLSP |
| | (101-200) | QARPNNAYTAMSDSYLPSYYSPSIGFSYSLGEAAWSTGGDT |
| | | AMPYLTSYGQLSNGEPHFLPDAMFGQPGALGSTPFLGQHGF |
| | | NFFPSGIDFSAWGNNSSQGQSTQSSGYSSNYAYAPSSLGGA |
| | | MIDGQSAFANETLNKAPGMNTIDQGMAA (SEQ ID NO: 17) |
| | | GS |
| | Flag tag | DYKDDDDK (SEQ ID NO: 14) |
| CIRTS-18X | ORF5 | MDDPSFLTGRSTYAKRRRARRMNVCKCGAILHNNKDCRSS |
| | | TISGHKLDRLRFVKEGRVALEGETPVYRTWVKWVETEYHIN |
| | | ILETSDDEE (SEQ ID NO: 5) |
| | GGS₆ | GGSGGSGGSGGSGGSGGS (SEQ ID NO: 6) |
| | PP7 | MAKTIVLAVGEATRTLTEIQSTADRQIFEEKVGPLVGRLRLT |
| | | ASLRQNGAKTAYRVNLKLDQADVVDASTSVAGELPKVRYT |
| | | QVWSHDVTIVANSTEASRKSLYDLTKSLVATSQVEDLVVNL |
| | | VPLGRSLEGGSGGMAKTIVLAVGEATRTLTEIQSTADRQIFE |
| | | EKVGPLVGRLRLTASLRQNGAKTAYRVNLKLDQADVVDAS |
| | | TSVAGELPKVRYTQVWSHDVTIVANSTEASRKSLYDLTKSL |
| | | VATSQVEDLVVNLVPLGRSLE (SEQ ID NO: 125) |
| | NES | LQLPPLERLTL (SEQ ID NO: 8) |
| | GGS₆ | GGSGGSGGSGGSGGSGGS (SEQ ID NO: 6) |
| | Y2 | DAMFGQPGALGSTPFLGQHGFNFFPSGIDFSAWGNNSSQGQ |
| | (100-200) | STQSSGYSSNYAYAPSSLGGAMIDGQSAFANETLNKAPGMN |
| | | TIDQGMAA (SEQ ID NO: 16) |
| | | GS |
| | Flag tag | DYKDDDDK (SEQ ID NO: 14) |

gRNA sequences used in this study (all gRNAs are expressed from a hU6 promoter)

| Name: | Description: | DNA | sequence |
|---|---|---|---|
| TBP- | 26-51 | TBP6.7 gRNA | GGCCAGATCTGAGCCTGGGAGCTCTCTGGCC |
| OT | | (TAR) | (SEQ ID NO: 26) |
| | | | TTATT |
| | | off-target λ | Tgacagcccacatggcattccacttatcactggcatcctt |
| | | phage | (SEQ ID NO: 27) |
| SLBP- | 24-18 | SLBP gRNA | CCAAAGGCTCTTCTCAGAGCCACCCA (SEQ ID |
| OT | | | NO: 28) |
| | | | TTATT |
| | | λ target | GTGATAAGTGGAATGCCATG (SEQ ID NO: 29) |
| PP7-NT | | PP7 non- | TAAGGAGTTTATATGGAAACCCTTA (SEQ ID |
| | | targeting gRNA | NO: 121) |
| | | λ phage | |
| | | | TTATT |
| | | λ phage | GTGATAAGTGGAATGCCATG (SEQ ID NO: 122) |

gRNA guiding sequencing; all expressed by the same hU6 promoter as TBP-OT

| Target name: | | Guiding Sequence: | Fig: |
|---|---|---|---|
| Fluciferase-40 | 18-20 | CAGGTCGACTCTAGACTCGAGGCTAGCGAGCTCGTT | 3 |
| | | TAAA (SEQ ID NO: 30) | |
| PPIB | 23-26 | CTTGGTGCTCTCCACCTTCCGCACCACCTCCATGCC | 4 |
| | | CTCT (SEQ ID NO: 31) | |
| SMARCA4 | 23-25 | CCGATGCGGTGGGCTCGGTCCTGCGCTTGCAGGTCC | 4 |
| | | TGGT (SEQ ID NO: 32) | |
| NFKB1 | 22-67 | GCCTCCACCAGCTCTCTGACTGTACCCCCAGAGACC | 4 |
| | | TCAT (SEQ ID NO: 33) | |
| NRAS | 25-30 | AAGCATCTTCAACACCCTGTCTGGTCTTGGCTGAGG | 4 |
| | | TTTC (SEQ ID NO: 34) | |
| B4GALTN1 | 24-50 | CTTCGCACCGCAGCGCAGCGCGGCTCAGCTCCCGG | 4 |
| | | CTCGT (SEQ ID NO: 35) | |
| Fluciferase-20 | | CTAGACTCGAGGCTAGCGAG (SEQ ID NO: 36) | 8 |
| Fluciferase-30 | | CGACTCTAGACTCGAGGCTAGCGAGCTCGT (SEQ ID | 8 |
| | | NO: 37) | |
| KRAS-2 | 22-45 | CTTGTGGTAGTTGGAGCTGA (SEQ ID NO: 116) | 25B |
| KRAS-3 | 22-53 | AGTTGGAGCTGATGGCGTAG (SEQ ID NO: 117) | 25B |
| KRAS-4 | 22-54 | GTTGGAGCTGATGGCGTAGG (SEQ ID NO: 118) | 25B |
| KRAS-5 | 22-65 | TGGCGTAGGCAAGAGTGCCT (SEQ ID NO: 119) | 25B |
| KRAS-6 | 22-66 | GGCGTAGGCAAGAGTGCCTT (SEQ ID NO: 120) | 25B |
| SMARCA4-1 | 27-67 | ggccctggcccttcccctggagccatgctgggccctagcc (SEQ ID NO: 38) | 11C |
| SMARCA4-2 | 27-71 | gcccaaccccatttaaccagaaccagctgcaccagctcag (SEQ ID NO: 39) | 11C |
| SMARCA4-3 | 27-74 | cctcccaagccctggcctgaaggacccatggcgaatgctg (SEQ ID NO: 40) | 11C |
| SMARCA4-4 | 27-75 | cgtcccacccgccgcctcgcccgtgatgccaccgcagacc (SEQ ID | 11C |
| | | NO: 41) | |
| SMARCA4-5 | 27-78 | ggaggtggtggtgtgcatgcggagggacacagcgctggag (SEQ ID | 11C |
| | | NO: 42) | |
| SMARCA4-6 | 27-80 | Tcacaggcaaaatccagaagctgaccaaggcagtggccac (SEQ ID | 11C |
| | | NO: 43) | |
| SMARCA4-7 | 27-81 | catggctgaagatgaggaggggtaccgcaagctcatcgac (SEQ ID | 11C |
| | | NO: 44) | |
| SMARCA4-8 | 28-02 | ctctggacgagaccagccagatgagcgacctcccggtgaa (SEQ ID | 11C |
| | | NO: 45) | |
| SMARCA4-9 | 28-04 | cccaccctgcccgtggaggagaagaagaagattccagatc (SEQ ID | 11C |
| | | NO: 46) | |
| SMARCA4-10 | 28-05 | aatatggcgtgtcccaggcccttgcacgtggcctgcagtc (SEQ ID NO: 47) | 11C |
| SMARCA4-11 | 28-07 | ctcatcacgtacctcatggagcacaaacgcatcaatgggc (SEQ ID NO: 48) | 11C |
| SMARCA4- | 28-08 | tggtgaaggtgtcttacaagggatccccagcagcaagacg (SEQ ID NO: 49) | 11C |
| 12 | | | |
| SMARCA4- | 28-10 | accccgccgcctgctgctgacgggcacaccgctgcagaac (SEQ ID | 11C |
| 13 | | NO: 50) | |
| SMARCA4- | 28-11 | cagtggtttaacgcaccctttgccatgaccggggaaaagg (SEQ ID NO: 51) | 11C |
| 14 | | | |
| SMARCA4- | 28-12 | acgactcaagaaggaagtcgaggcccagttgcccgaaaag (SEQ ID | 11C |
| 15 | | NO: 52) | |
| SMARCA4- | 28-13 | gtgctgctgactgatggctccgagaaggacaagaagggca (SEQ ID | 11C |
| 16 | | NO: 53) | |
| SMARCA4- | 28-16 | ggaaccacgaaggcggaggaccggggcatgctgctgaaaa (SEQ ID | 11C |
| 17 | | NO: 54) | |
| SMARCA4- | 28-17 | tccagtcggcagacactgtgatcatttttgacagcgactg (SEQ ID NO: 55) | 11C |
| 18 | | | |
| SMARCA4- | 28-20 | cgccagcgggcgtcaaccccgacttggaggagccacctct (SEQ ID | 11C |
| 19 | | NO: 56) | |
| SMARCA4- | 28-22 | gcggaggtggagcggctgacctgtgaggaggaggaggaga (SEQ ID | 11C |
| 20 | | NO: 57) | |
| SMARCA4- | 28-26 | tgatcaagtacaaggacagcagcagtggacgtcagctcag (SEQ ID NO: 58) | 11C |
| 21 | | | |
| SMARCA4- | 28-27 | cttcaagaagataaaggagcgcattcgcaaccacaagtac (SEQ ID NO: 59) | 11C |
| 22 | | | |
| SMARCA4- | 28-30 | agggtcccgagccaagccggtcgtgagtgacgatgacagt (SEQ ID | 11C |
| 23 | | NO: 60) | |
| SMARCA4- | 28-32 | tatttatacagcagagaagctgtaggactgtttgtgactg (SEQ ID NO: 61) | 11C |
| 24 | | | |
| SMARCA4- | 28-33 | ggggaacacacgatacctgtttttcttttccgttgctggc (SEQ ID NO: 62) | 11C |
| 25 | | | |

RNA oligonucleotides used for in vitro characterization:

| Gene | Oligo | | |
|---|---|---|---|
| Name: | Name: | Forward: | Figure: |
| R1 | ssRNA | ATAGGCCAGTGAATTCGAGCTCGAATATGGATTACTT | 2A |
| | target | GGTAGAACAGCAATCTACGCCGGAAGCATAAAG (SEQ | |
| | for | ID NO: 63) | |
| | EMSA | | |
| R2 | ssRNA | GGCCAGTGAATTCGAGCTCGGTACCCGGGGATCCTCT | 2C |
| | target | AGAAATATGGATTACTTGGTAGAACAGCAATCTACTC | |
| | for | GACCTGCAGGCATGCAAGCTTGGCGTAATCATGGTCA | |
| | cleavage | TAGCTGTTTCCTGTGTTTATCCGCTCACAATTCCACAC | |
| | assay | AACATACGAGCCGGAAGCATAAAG (SEQ ID NO: 64) | |
| R3 | gRNA | GGCCAGATCTGAGCCTGGGAGCTCTCTGGCCCagccacca | 2A |
| | for | cccacagagccgccaccaga (SEQ ID NO: 65) | |
| | EMSA | | |
| R4 | gRNA | GGCCAGATCTGAGCCTGGGAGCTCTCTGGCCCTAGAT | 2C |
| | for | TGCTGTTCTACCAAGTAATCCAT (SEQ ID NO: 66) | |
| | cleavage | | |
| | assay | | |

qPCR Primers:

| | | SEQ | | SEQ |
|---|---|---|---|---|
| Gene | | ID | | ID |
| Name: | Forward: | NO: | Reverse: | NO: |
| GAPDH | GTCTCCTCTGACTTCA | 67 | ACCACCCTGTTGCTGTAGCC | 68 |
| | ACAGCG | | AA | |
| PPIB | AACGCAGGCAAAGAC | 69 | TCTGTCTTGGTGCTCTCCAC | 70 |
| | ACCAACG | | CT | |
| Fluciferase | AGGTTACAACCGCCAA | 71 | ATGAGAATCTCGCGGATCTT | 72 |
| | GAAGC | | G | |
| PPIB | AACGCAGGCAAAGAC | 73 | TCTGTCTTGGTGCTCTCCAC | 74 |
| | ACCAACG | | CT | |
| NFKB1 | GCAGCACTACTTCTTG | 75 | TCTGCTCCTGAGCATTGACG | 76 |
| | ACCACC | | TC | |
| NRAS | GAAACCTCAGCCAAG | 77 | GGCAATCCCATACAACCCTG | 78 |
| | ACCAGAC | | AG | |
| B4GALTN1 | TGAGGCTGCTTTCACT | 79 | GAGGAAGGTCTTGGTGGCA | 80 |
| | ATCCGC | | ATC | |
| SMARCA4 | CAAAGACAAGCACAT | 81 | GCCACATAGTGCGTGTTGAG | 82 |
| | CCTCGCC | | CA | |
| MALAT1 | GAAGAAGGAAGGAGC | 126 | CCTGCTACCTTCATCACCAA | 127 |
| | GCTAA | | | |

RNA hairpin structure:
(SEQ ID NO: 83)
GGCGUCCCUCCCGAAGCUGCGCGCUCGGUCGAACAGGACGACC
Nucleolin recognition sequence:
(SEQ ID NO: 84)
UCCCGA.
RNA hairpin structure:
(SEQ ID NO: 85)
GGCCGAAAUCCCGAAUGAGGCC
and
(SEQ ID NO: 86)
GGAUGCCUCCCGAGUGCAUCC.

| Name: | Description: | Polypeptide sequence |
|---|---|---|
| CIRTS-0 | ORF5- | MDDPSFLTGRSTYAKRRRARRMNVCKCGAILHNNKDCRSS |
| | TBP6.7- | TISGHKLDRLRFVKEGRVALEGETPVYRTWVKWVETEYHI |
| | GGS₆-dead | NILETSDDEEGGSGGSGGSGGSGGSGGSMAVPETRPNHTIY |
| | Pin domain | INNLNSKIKKDELKKSLYAIFSQFGQILDILVPRQRTPRGQAF |
| | | VIFKEVSSATNALRSMQGFPFYDKPMRIQYAKTDKRIPAKM |
| | | KGTFVGSLQLPPLERLTLGGSGGSGGSGGSGGSGGSMELEI |
| | | RPLFLVPDTNGFIDHLASLARLLESRKYILVVPLIVINELDGL |
| | | AKGQETDHRAGGYARVVQEKARKSIEFLEQRFESRDSCLR |
| | | ALTSRGNELESIAFRSEDITGQLGNNADLILSCCLHYCKDKA |
| | | KDFMPASKEEPIRLLREVVLLTDDRNLRVKALTRNVPVRDI |
| | | PAFLTWAQVGGSDYKDHDGDYKDHDIDYKDDDDK (SEQ |
| | | ID NO: 87) |
| CIRTS-1 | ORF5- | MDDPSFLTGRSTYAKRRRARRMNVCKCGAILHNNKDCRSS |
| | TBP6.7- | TISGHKLDRLRFVKEGRVALEGETPVYRTWVKWVETEYHI |
| | GGS₆-Pin | NILETSDDEEGGSGGSGGSGGSGGSGGSMAVPETRPNHTIY |
| | domain-NLS | INNLNSKIKKDELKKSLYAIFSQFGQILDILVPRQRTPRGQAF |
| | | VIFKEVSSATNALRSMQGFPFYDKPMRIQYAKTDKRIPAKM |
| | | KGTFVGGSGGSGGSGGSGGSGGSMELEIRPLFLVPDTNGFI |
| | | DHLASLARLLESRKYILVVPLIVINELDGLAKGQETDHRAG |
| | | GYARVVQEKARKSIEFLEQRFESRDSCLRALTSRGNELESIA |
| | | FRSEDITGQLGNNDDLILScCLHYCKDKAKDFMPASKEEPI |
| | | RLLREVVLLTDDRNLRVKALTRNVPVRDIPAFLTWAQVGK |
| | | RPAATKKAGQAKKKKGSDYKDDDDK (SEQ ID NO: 88) |
| CIRTS-2 | ORF5- | MDDPSFLTGRSTYAKRRRARRMNVCKCGAILHNNKDCRSS |
| | TBP6.7- | TISGHKLDRLRFVKEGRVALEGETPVYRTWVKWVETEYHI |
| | GGS₆- | NILETSDDEEGGSGGSGGSGGSGGSGGSMAVPETRPNHTIY |
| | NYTHDF1 | INNLNSKIKKDELKKSLYAIFSQFGQILDILVPRQRTPRGQAF |
| | | VIFKEVSSATNALRSMQGFPFYDKPMRIQYAKTDKRIPAKM |
| | | KGTFVGSLQLPPLERLTLGGSGGSGGSGGSGGSGGSMSATS |
| | | VDTQRTKGQDNKVQNGSLHQKDTVHDNDFEPYLTGQSNQ |
| | | SNSYPSMSDPYLSSYYPPSIGFPYSLNEAPWSTAGDPPIPYLT |
| | | TYGQLSNGDHHFMHDAVFGQPGGLGNNIYQHRFNFFPENP |
| | | AFSAWGTSGSQGQQTQSSAYGSSYTYPPSSLGGTVVDGQP |
| | | GFHSDTLSKAPGMNSLEQGMVGLKIGDVSSSAVKTVGSVV |
| | | SSVALTGVLSGNGGTNVNMPVSKPTSWAAIASKPAKPQPK |
| | | MKTKSGPVIVIGGGLPPPPIKHNMDIGTWDNKGPVPKAPVPQ |
| | | QAPSPQAAPQPQQVAQPLPAQPPALAQPQYQSPQQPPQTR |
| | | WVAPRNRNAAFGQSGGAGSDSNSPGNVQPNSAPSVESGSD |
| | | YKDDDDK (SEQ ID NO: 89) |
| CIRTS-3t | ORF5- | MDDPSFLTGRSTYAKRRRARRMNVCKCGAILHNNKDCRSS |
| | TBP6.7- | TISGHKLDRLRFVKEGRVALEGETPVYRTWVKWVETEYHI |
| | GGS₆- | NILETSDDEEGGSGGSGGSGGSGGSGGSMAVPETRPNHTIY |
| | YTHDF2 | INNLNSKIKKDELKKSLYAIFSQFGQILDILVPRQRTPRGQAF |
| | (100-200) | VIFKEVSSATNALRSMQGFPFYDKPMRIQYAKTDKRIPAKM |
| | | KGTFVGSLQLPPLERLTLGGSGGSGGSGGSGGSGGSDAMF |
| | | GQPGALGSTPFLGQHGFNFFPSGIDFSAWGNNSSQGQSTQS |
| | | SGYSSNYAYAPSSLGGAMIDGQSAFANETLNKAPGMNTID |
| | | QGMAAGSDYKDDDDK (SEQ ID NO: 90) |
| CIRTS-3 | ORF5- | MDDPSFLTGRSTYAKRRRARRMNVCKCGAILHNNKDCRSS |
| | TBP6.7- | TISGHKLDRLRFVKEGRVALEGETPVYRTWVKWVETEYHI |
| | GGS₆- | NILETSDDEEGGSGGSGGSGGSGGSGGSMAVPETRPNHTIY |
| | YTHDF2 | INNLNSKIKKDELKKSLYAIFSQFGQILDILVPRQRTPRGQAF |
| | (1-200) | VIFKEVSSATNALRSMQGFPFYDKPMRIQYAKTDKRIPAKM |
| | | KGTFVGSLQLPPLERLTLGGSGGSGGSGGSGGSGGSMSASS |
| | | LLEQRPKGQGNKVQNGSVHQKDGLNDDDFEPYLSPQARP |
| | | NNAYTAMSDSYLPSYYSPSIGFSYSLGEAAWSTGGDTAMP |
| | | YLTSYGQLSNGEPHFLPDAMFGQPGALGSTPFLGQHGFNFF |
| | | PSGIDFSAWGNNSSQGQSTQSSGYSSNYAYAPSSLGGAMID |
| | | GQSAFANETLNKAPGMNTIDQGMAAGSDYKDDDDK (SEQ |
| | | ID NO: 91) |
| CIRTS-4t | ORF5-SLBP- | MDDPSFLTGRSTYAKRRRARRMNVCKCGAILHNNKDCRSS |
| | GGS₆- | TISGHKLDRLRFVKEGRVALEGETPVYRTWVKWVETEYHI |
| | YTHDF2 | NILETSDDEEGGSGGSGGSGGSGGSGGSADFETDESVLMRR |
| | (100-200) | QKQINYGKNTIAYDRYIKEVPRHLRQPGIHPKTPNKFKKYS |
| | | RRSWDQQIKLWKVALHFWDLQLPPLERLTLGGSGGSGGSG |
| | | GSGGSGGSDANIFGQPGALGSTPFLGQHGFNFFPSGIDFSAW |
| | | GNNSSQGQSTQSSGYSSNYAYAPSSLGGAMIDGQSAFANE |
| | | TLNKAPGMNTIDQGMAAGSDYKDDDDK (SEQ ID NO: 92) |
| CIRTS-4 | ORF5-SLBP- | MDDPSFLTGRSTYAKRRRARRMNVCKCGAILHNNKDCRSS |
| | GGS₆- | TISGHKLDRLRFVKEGRVALEGETPVYRTWVKWVETEYHI |
| | YTHDF2 | NILETSDDEEGGSGGSGGSGGSGGSGGSADFETDESVLMRR |
| | (1-200) | QKQINYGKNTIAYDRYIKEVPRHLRQPGIHPKTPNKFKKYS |
| | | RRSWDQQIKLWKVALHFWDLQLPPLERLTLGGSGGSGGSG |
| | | GSGGSGGSMSASSLLEQRPKGQGNKVQNGSVHQKDGLND |
| | | DDFEPYLSPQARPNNAYTAMSDSYLPSYYSPSIGFSYSLGE |
| | | AAWSTGGDTAMPYLTSYGQLSNGEPHFLPDAMFGQPGAL |
| | | GSTPFLGQHGFNFFPSGIDFSAWGNNSSQGQSTQSSGYSSN |
| | | YAYAPSSLGGAMIDGQSAFANETLNKAPGMNTIDQGMAA |
| | | GSDYKDDDDK (SEQ ID NO: 93) |
| CIRTS-5t | HBEGF- | MRVTLSSKPQALATPNKEEHGKRKKKGKGLGKKRDPCLR |
| | TBP6.7- | KYKDFCIHGECKYVKELRAPSCICHPGYHGERCHGLSGGS |
| | GGS₆- | GGSGGSGGSGGSGGSMAVPETRPNHTIYINNLNSKIKKDEL |
| | YTHDF2 | KKSLYAIFSQFGQILDILVPRQRTPRGQAFVIFKEVSSATNA |
| | (100-200) | LRSMQGFPFYDKPMRIQYAKTDKRIPAKMKGTFVGSLQLP |
| | | PLERLTLGGSGGSGGSGGSGGSGGSDAMFGQPGALGSTPFL |
| | | GQHGFNFFPSGIDFSAWGNNSSQGQSTQSSGYSSNYAYAPS |
| | | SLGGAMIDGQSAFANETLNKAPGMNTIDQGMAAGSDYKD |
| | | DDDK (SEQ ID NO: 94) |
| CIRTS-5 | HBEGF- | MRVTLSSKPQALATPNKEEHGKRKKKGKGLGKKRDPCLR |
| | TBP6.7- | KYKDFCIHGECKYVKELRAPSCICHPGYHGERCHGLSGGS |
| | GGS₆- | GGSGGSGGSGGSGGSMAVPETRPNHTIYINNLNSKIKKDEL |
| | YTHDF2 | KKSLYAIFSQFGQILDILVPRQRTPRGQAFVIFKEVSSATNA |
| | (1-200) | LRSMQGFPFYDKPMRIQYAKTDKRIPAKMKGTFVGSLQLP |
| | | PLERLTLGGSGGSGGSGGSGGSGGSMSASSLLEQRPKGQG |
| | | NKVQNGSVHQKDGLNDDDFEPYLSPQARPNNAYTAMSDS |
| | | YLPSYYSPSIGFSYSLGEAAWSTGGDTAMPYLTSYGQLSNG |
| | | EPHFLPDAMFGQPGALGSTPFLGQHGFNFFPSGIDFSAWGN |
| | | NSSQGQSTQSSGYSSNYAYAPSSLGGAMIDGQSAFANETLN |
| | | KAPGMNTIDQGMAAGSDYKDDDDK (SEQ ID NO: 95) |
| CIRTS-6t | β-defensin- | MGIINTLQKYYCRVRGGRCAVLSCLPKEEQIGKCSTRGRKC |
| | TBP6.7- | CRRKKGGSGGSGGSGGSGGSGGSMAVPETRPNHTIYINNL |
| | GGS₆- | NSKIKKDELKKSLYAIFSQFGQILDILVPRQRTPRGQAFVIFK |
| | YTHDF2 | EVSSATNALRSMQGFPFYDKPMRIQYAKTDKRIPAKMKGT |
| | (100-200) | FVGSLQLPPLERLTLGGSGGSGGSGGSGGSGGSDAMFGQP |
| | | GALGSTPFLGQHGFNFFPSGIDFSAWGNNSSQGQSTQSSGY |
| | | SSNYAYAPSSLGGAMIDGQSAFANETLNKAPGMNTIDQGM |
| | | AAGSDYKDDDDK (SEQ ID NO: 96) |
| CIRTS-6 | β-defensin- | MGIINTLQKYYCRVRGGRCAVLSCLPKEEQIGKCSTRGRKC |
| | TBP6.7- | CRRKKGGSGGSGGSGGSGGSGGSMAVPETRPNHTIYINNL |
| | GGS₆- | NSKIKKDELKKSLYAIFSQFGQILDILVPRQRTPRGQAFVIFK |
| | YTHDF2 | EVSSATNALRSMQGFPFYDKPMRIQYAKTDKRIPAKMKGT |
| | (1-200) | FVGSLQLPPLERLTLGGSGGSGGSGGSGGSGGSMSASSLLE |
| | | QRPKGQGNKVQNGSVHQKDGLNDDDFEPYLSPQARPNNA |
| | | YTAMSDSYLPSYYSPSIGFSYSLGEAAWSTGGDTAMPYLTS |
| | | YGQLSNGEPHFLPDAMFGQPGALGSTPFLGQHGFNFFPSGI |
| | | DFSAWGNNSSQGQSTQSSGYSSNYAYAPSSLGGAMIDGQS |
| | | AFANETLNKAPGMNTIDQGMAAGSDYKDDDDK (SEQ ID |
| | | NO: 97) |
| CIRTS-7 | β-defensin- | MGIINTLQKYYCRVRGGRCAVLSCLPKEEQIGKCSTRGRKC |
| | SLBP-GGS₆- | CRRKKGGSGGSGGSGGSGGSGGSADFETDESVLMRRQKQI |
| | YTHDF2 | NYGKNTIAYDRYIKEVPRHLRQPGIHPKTPNKFKKYSRRSW |
| | (1-200) | DQQIKLWKVALHFWDLQLPPLERLTLGGSGGSGGSGGSGG |
| | | SGGSMSASSLLEQRPKGQGNKVQNGSVHQKDGLNDDDFE |
| | | PYLSPQARPNNAYTAMSDSYLPSYYSPSIGFSYSLGEAAWS |
| | | TGGDTAMPYLTSYGQLSNGEPHFLPDAMFGQPGALGSTPF |
| | | LGQHGFNFFPSGIDFSAWGNNSSQGQSTQSSGYSSNYAYAP |
| | | SSLGGAMIDGQSAFANETLNKAPGMNTIDQGMAAGSDYK |
| | | DDDDK (SEQ ID NO: 98) |
| CIRTS-8 | TBP6.7- | MAVPETRPNHTIYINNLNSKIKKDELKKSLYAIFSQFGQILDI |
| | GGS₆-Pin | LVPRQRTPRGQAFVIFKEVSSATNALRSMQGFPFYDKPMRI |
| | domain-NES | QYAKTDKRIPAKMKGTFVGSLQLPPLERLTLGGSGGSGGS |
| | | GGSGGSGGSMELEIRPLFLVPDTNGFIDHLASLARLLESRKY |
| | | ILVVPLIVINELDGLAKGQETDHRAGGYARVVQEKARKSIE |
| | | FLEQRFESRDSCLRALTSRGNELESIAFRSEDITGQLGNNDD |
| | | LILSCCLHYCKDKAKDFMPASKEEPIRLLREVVLLTDDRNL |
| | | RVKALTRNVPVRDIPAFLTWAQVGGSDYKDDDDK (SEQ |
| | | ID NO: 99) |
| CIRTS-9 | ORF5- | MDDPSFLTGRSTYAKRRRARRMNVCKCGAILHNNKDCRSS |
| | TBP6.7- | TISGHKLDRLRFVKEGRVALEGETPVYRTWVKWVETEYHI |
| | GGS₆-Pin | NILETSDDEEGGSGGSGGSGGSGGSGGSMAVPETRPNHTIY |
| | domain-NES | INNLNSKIKKDELKKSLYAIFSQFGQILDILVPRQRTPRGQAF |
| | | VIFKEVSSATNALRSMQGFPFYDKPMRIQYAKTDKRIPAKM |
| | | KGTFVGSLQLPPLERLTLGGSGGSGGSGGSGGSGGSMELEI |
| | | RPLFLVPDTNGFIDHLASLARLLESRKYILVVPLIVINELDGL |
| | | AKGQETDHRAGGYARVVQEKARKSIEFLEQRFESRDSCLR |
| | | ALTSRGNELESIAFRSEDITGQLGNNDDLILSCCLHYCKDKA |
| | | KDFMPASKEEPIRLLREVVLLTDDRNLRVKALTRNVPVRDI |
| | | PAFLTWAQVGGSDYKDDDDK (SEQ ID NO: 100) |
| CIRTS- | ORF5- | MDDPSFLTGRSTYAKRRRARRMNVCKCGAILHNNKDCRSS |
| 10 | TBP6.7- | TISGHKLDRLRFVKEGRVALEGETPVYRTWVKWVETEYHI |
| | GGS₃-Pin | NILETSDDEEGGSGGSGGSGGSGGSGGSMAVPETRPNHTIY |
| | domain | INNLNSKIKKDELKKSLYAIFSQFGQILDILVPRQRTPRGQAF |
| | | VIFKEVSSATNALRSMQGFPFYDKPMRIQYAKTDKRIPAKM |
| | | KGTFVGSLQLPPLERLTLGGSGGSGGSMELEIRPLFLVPDTN |
| | | GFIDHLASLARLLESRKYILVVPLIVINELDGLAKGQETDHR |
| | | AGGYARVVQEKARKSIEFLEQRFESRDSCLRALTSRGNELE |
| | | SIAFRSEDITGQLGNNDDLILSCCLHYCKDKAKDFMPASKE |
| | | EPIRLLREVVLLTDDRNLRVKALTRNVPVRDIPAFLTWAQV |
| | | GGSDYKDDDDK (SEQ ID NO: 101) |
| CIRTS- | ORF5- | MDDPSFLTGRSTYAKRRRARRMNVCKCGAILHNNKDCRSS |
| 11 | TBP6.7-L8- | TISGHKLDRLRFVKEGRVALEGETPVYRTWVKWVETEYHI |
| | Pin domain | NILETSDDEEGGSGGSGGSGGSGGSGGSMAVPETRPNHTIY |
| | | INNLNSKIKKDELKKSLYAIFSQFGQILDILVPRQRTPRGQAF |
| | | VIFKEVSSATNALRSMQGFPFYDKPMRIQYAKTDKRIPAKM |
| | | KGTFVGSLQLPPLERLTLSGSETPGTSESATPESMELEIRPLF |
| | | LVPDTNGFIDHLASLARLLESRKYILVVPLIVINELDGLAKG |
| | | QETDHRAGGYARVVQEKARKSIEFLEQRFESRDSCLRALTS |
| | | RGNELESIAFRSEDITGQLGNNDDLILSCCLHYCKDKAKDF |
| | | MPASKEEPIRLLREVVLLTDDRNLRVKALTRNVPVRDIPAF |
| | | LTWAQVGGSDYKDDDDK (SEQ ID NO: 102) |
| CIRTS- | ORF5- | MDDPSFLTGRSTYAKRRRARRMNVCKCGAILHNNKDCRSS |
| 12 | TBP6.7- | TISGHKLDRLRFVKEGRVALEGETPVYRTWVKWVETEYHI |
| | helical-Pin | NILETSDDEEGGSGGSGGSGGSGGSGGSMAVPETRPNHTIY |
| | domain-NES | INNLNSKIKKDELKKSLYAIFSQFGQILDILVPRQRTPRGQAF |
| | | VIFKEVSSATNALRSMQGFPFYDKPMRIQYAKTDKRIPAKM |
| | | KGTFVGSLQLPPLERLTLGGSGEEEEKKKQQEEEAERLRRI |
| | | QEEMEKERKRREEDEQRRRKEEEERRMKLEMEAKRKQEE |
| | | EERKKREDDEKRKKKGSGGSMELEIRPLFLVPDTNGFIDHL |
| | | ASLARLLESRKYILVVPLIVINELDGLAKGQETDHRAGGYA |
| | | RVVQEKARKSIEFLEQRFESRDSCLRALTSRGNELESIAFRS |
| | | EDITGQLGNNDDLILSCCLHYCKDKAKDFMPASKEEPIRLL |
| | | REVVLLTDDRNLRVKALTRNVPVRDIPAFLTWAQVGGSDY |
| | | KDDDDK (SEQ ID NO: 103) |
| CIRTS- | ORF5- | MDDPSFLTGRSTYAKRRRARRMNVCKCGAILHNNKDCRSS |
| 13 | TBP6.7-L8- | TISGHKLDRLRFVKEGRVALEGETPVYRTWVKWVETEYHI |
| | Y2(100-200) | NILETSDDEEGGSGGSGGSGGSGGSGGSMAVPETRPNHTIY |
| | | INNLNSKIKKDELKKSLYAIFSQFGQILDILVPRQRTPRGQAF |
| | | VIFKEVSSATNALRSMQGFPFYDKPMRIQYAKTDKRIPAKM |
| | | KGTFVGSLQLPPLERLTLSGSETPGTSESATPESDAMFGQPG |
| | | ALGSTPFLGQHGFNFFPSGIDFSAWGNNSSQGQSTQSSGYS |
| | | SNYAYAPSSLGGAMIDGQSAFANETLNKAPGMNTIDQGM |
| | | AAGSDYKDDDDK (SEQ ID NO: 104) |
| CIRTS- | ORF5- | MDDPSFLTGRSTYAKRRRARRMNVCKCGAILHNNKDCRSS |
| 14 | TBP6.7- | TISGHKLDRLRFVKEGRVALEGETPVYRTWVKWVETEYHI |
| | helical- | NILETSDDEEGGSGGSGGSGGSGGSGGSMAVPETRPNHTIY |
| | Y2(100-200) | INNLNSKIKKDELKKSLYAIFSQFGQILDILVPRQRTPRGQAF |
| | | VIFKEVSSATNALRSMQGFPFYDKPMRIQYAKTDKRIPAKM |
| | | KGTFVGSLQLPPLERLTLGGSGEEEEKKKQQEEEAERLRRI |
| | | QEEMEKERKRREEDEQRRRKEEEERRMKLEMEAKRKQEE |
| | | EERKKREDDEKRKKKGSGGSDAMFGQPGALGSTPFLGQH |
| | | GFNFFPSGIDFSAWGNNSSQGQSTQSSGYSSNYAYAPSSLG |
| | | GAMIDGQSAFANETLNKAPGMNTIDQGMAAGSDYKDDDD |
| | | K (SEQ ID NO: 105) |
| MBP- | MBP-ORF5- | MDDPSFLTGRSTYAKRRRARRMNVCKCGAILHNNKDCRSS |
| CIRTS-1 | TBP6.7- | TISGHKLDRLRFVKEGRVALEGETPVYRTWVKWVETEYHI |
| | GGS₆-dead | NILETSDDEEGGSGGSGGSGGSGGSGGSMAVPETRPNHTIY |
| | Pindomain | INNLNSKIKKDELKKSLYAIFSQFGQILDILVPRQRTPRGQAF |
| | | VIFKEVSSATNALRSMQGFPFYDKPMRIQYAKTDKRIPAKM |
| | | KGTFVGSLQLPPLERLTLGGSGGSGGSGGSGGSGGSMELEI |
| | | RPLFLVPDTNGFIDHLASLARLLESRKYILVVPLIVINELDGL |
| | | AKGQETDHRAGGYARVVQEKARKSIEFLEQRFESRDSCLR |
| | | ALTSRGNELESIAFRSEDITGQLGNNDDLILSCCLHYCKDKA |
| | | KDFMPASKEEPIRLLREVVLLTDDRNLRVKALTRNVPVRDI |
| | | PAFLTWAQVGSSGHHHHHH (SEQ ID NO: 106) |
| CIRTS- | β-defensin 3- | MGIINTLQKYYCRVRGGRCAVLSCLPKEEQIGKCSTRGRKC |
| 7X | TBP6.7- | CRRKKGGSGGSGGSGGSGGSGGSMAVPETRPNHTIYINNL |
| | GGS₆- | NSKIKKDELKKSLYAIFSQFGQILDILVPRQRTPRGQAFVIFK |
| | hADAR | EVSSATNALRSMQGFPFYDKPMRIQYAKTDKRIPAKMKGT |
| | (299-701) | FVGGSGGSGGSGGSGGSGGSLHLDQTPSRQPIPSEGLQLHL |
| | | PQVLADAVSRLVLGKFGDLTDNFSSPHARRKVLAGVVMTT |
| | | GTDVKDAKVISVSTGTKCINGEYMSDRGLALNDCHAEIISR |
| | | RSLLRFLYTQLELYLNNKDDQKRSIFQKSERGGFRLKENVQ |
| | | FHLYISTSPCGDARIFSPHEPILEEPADRHPNRKARGQLRTKI |
| | | ESGEGTIPVRSNASIQTWDGVLQGERLLTMSCSDKIARWNV |
| | | VGIQGSLLSIFVEPIYFSSIILGSLYHGDHLSRAMYQRISNIED |
| | | LPPLYTLNKPLLSGISNAEARQPGKAPNFSVNWTVGDSAIE |
| | | VINATTGKDELGRASRLCKHALYCRWMRVHGKVPSHLLR |
| | | SKITKPNVYHESKLAAKEYQAAKARLFTAFIKAGLGAWVE |
| | | KPTEQDQFSLTPKRPAATKKAGQAKKKKGSDYKDDDDK |
| | | (SEQ ID NO: 128) |
| CIRTS- | β-defensin 3- | MGIINTLQKYYCRVRGGRCAVLSCLPKEEQIGKCSTRGRKC |
| 8X | TBP6.7- | CRRKKGGSGGSGGSGGSGGSGGSMAVPETRPNHTIYINNL |
| | GGS₆-hADAR | NSKIKKDELKKSLYAIFSQFGQILDILVPRQRTPRGQAFVIFK |
| | (299-701) | EVSSATNALRSMQGFPFYDKPMRIQYAKTDKRIPAKMKGT |
| | E488Q | FVGGSGGSGGSGGSGGSGGSLHLDQTPSRQPIPSEGLQLHL |
| | | PQVLADAVSRLVLGKFGDLTDNFSSPHARRKVLAGVVMTT |
| | | GTDVKDAKVISVSTGTKCINGEYMSDRGLALNDCHAEIISR |
| | | RSLLRFLYTQLELYLNNKDDQKRSIFQKSERGGFRLKENVQ |
| | | FHLYISTSPCGDARIFSPHEPILEEPADRHPNRKARGQLRTKI |
| | | ESGQGTIPVRSNASIQTWDGVLQGERLLTMSCSDKIARWN |
| | | VVGIQGSLLSIFVEPIYFSSIILGSLYHGDHLSRAMYQRISNIE |
| | | DLPPLYTLNKPLLSGISNAEARQPGKAPNFSVNWTVGDSAT |
| | | EVINATTGKDELGRASRLCKHALYCRWMRVHGKVPSHLL |
| | | RSKITKPNVYHESKLAAKEYQAAKARLFTAFIKAGLGAWV |
| | | EKPTEQDQFSLTPKRPAATKKAGQAKKKKGSDYKDDDDK |
| | | (SEQ ID NO: 129) |
| CIRTS- | βdefensin 3- | MGIINTLQKYYCRVRGGRCAVLSCLPKEEQIGKCSTRGRKC |
| 9X | SLBP-GGS₆- | CRRKKGGSGGSGGSGGSGGSGGSMAVPETRPNHTIYINNL |
| | NYTHDF1 | NSKIKKDELKKSLYAIFSQFGQILDILVPRQRTPRGQAFVIFK |
| | | EVSSATNALRSMQGFPFYDKPMRIQYAKTDKRIPAKMKGT |
| | | FVGSLQLPPLERLTLGGSGGSGGSGGSGGSGGSMSATSVDT |
| | | QRTKGQDNKVQNGSLHQKDTVHDNDFEPYLTGQSNQSNS |
| | | YPSMSDPYLSSYYPPSIGFPYSLNEAPWSTAGDPPIPYLTTY |
| | | GQLSNGDHHFMHDAVFGQPGGLGNNIYQHRFNFFPENPAF |
| | | SAWGTSGSQGQQTQSSAYGSSYTYPPSSLGGTVVDGQPGF |
| | | HSDTLSKAPGMNSLEQGMVGLKIGDVSSSAVKTVGSVVSS |
| | | VALTGVLSGNGGTNVNMPVSKPTSWAAIASKPAKPQPKM |
| | | KTKSGPVMGGGLPPPPIKHNMDIGTWDNKGPVPKAPVPQQ |
| | | APSPQAAPQPQQVAQPLPAQPPALAQPQYQSPQQPPQTRW |
| | | VAPRNRNAAFGQSGGAGSDSNSPGNVQPNSAPSVESGSDY |
| | | KDDDDK (SEQ ID NO: 130) |
| CIRTS- | β-defensin 3- | MGIINTLQKYYCRVRGGRCAVLSCLPKEEQIGKCSTRGRKC |
| 10X | SLBP-GGS₆- | CRRKKGGSGGSGGSGGSGGSGGSADFETDESVLMRRQKQI |
| | YTHDF2 | NYGKNTIAYDRYIKEVPRHLRQPGIHPKTPNKFKKYSRRSW |
| | (1-200) | DQQIKLWKVALHFWDLQLPPLERLTLGGSGGSGGSGGSGG |
| | | SGGSMSASSLLEQRPKGQGNKVQNGSVHQKDGLNDDDFE |
| | | PYLSPQARPNNAYTAMSDSYLPSYYSPSIGFSYSLGEAAWS |
| | | TGGDTAMPYLTSYGQLSNGEPHFLPDAMFGQPGALGSTPF |
| | | LGQHGFNFFPSGIDFSAWGNNSSQGQSTQSSGYSSNYAYAP |
| | | SSLGGAMIDGQSAFANETLNKAPGMNTIDQGMAAGSDYK |
| | | DDDDK (SEQ ID NO: 131) |
| CIRTS- | ORF5-PP7- | MDDPSFLTGRSTYAKRRRARRMNVCKCGAILHNNKDCRSS |
| 18X | Y2(100-200) | TISGHKLDRLRFVKEGRVALEGETPVYRTWVKWVETEYHI |
| | | NILETSDDEEGGSGGSGGSGGSGGSGGSMAKTIVLAVGEAT |
| | | RTLTEIQSTADRQIFEEKVGPLVGRLRLTASLRQNGAKTAY |
| | | RVNLKLDQADVVDASTSVAGELPKVRYTQVWSHDVTIVA |
| | | NSTEASRKSLYDLTKSLVATSQVEDLVVNLVPLGRSLEGGS |
| | | GGMAKTIVLAVGEATRTLTEIQSTADRQIFEEKVGPLVGRL |
| | | RLTASLRQNGAKTAYRVNLKLDQADVVDASTSVAGELPK |
| | | VRYTQVWSHDVTIVANSTEASRKSLYDLTKSLVATSQVED |
| | | LVVNLVPLGRSLELQLPPLERLTLGGSGGSGGSGGSGGSGG |
| | | SDAMFGQPGALGSTPFLGQHGFNFFPSGIDFSAWGNNSSQG |
| | | QSTQSSGYSSNYAYAPSSLGGAMIDGQSAFANETLNKAPG |
| | | MNTIDQGMAAGSDYKDDDDK (SEQ ID NO: 132) |

### VII. EXAMPLES

The following examples are included to demonstrate preferred embodiments of the disclosure. It should be appreciated by those of skill in the art that the techniques disclosed in the examples which follow represent techniques discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Programmable RNA-Guided RNA Effector Proteins Built from Human Parts

A. Introduction

To overcome the large size and microbial-derived nature of current RNA-targeting systems, the inventors present a CRISPR/Cas-inspired RNA targeting system (CIRTS), a general method for engineering programmable RNA effector proteins. The inventors show that CIRTS permits mining the human proteome for functional parts to build programmable RNA regulatory proteins. CIRTS is a ribonucleoprotein complex that uses Watson-Crick-Franklin base pair interactions to deliver protein cargo site-selectively in the transcriptome. The inventors show they can easily engineer CIRTS that deliver a range or regulatory proteins to transcripts, including nucleases for degradation, deadenylation regulatory machinery for degradation, or translational activation machinery for enhanced protein production. However, CIRTS are up to 5-fold smaller than the smallest current CRISPR/Cas systems and can be engineered entirely from human parts.

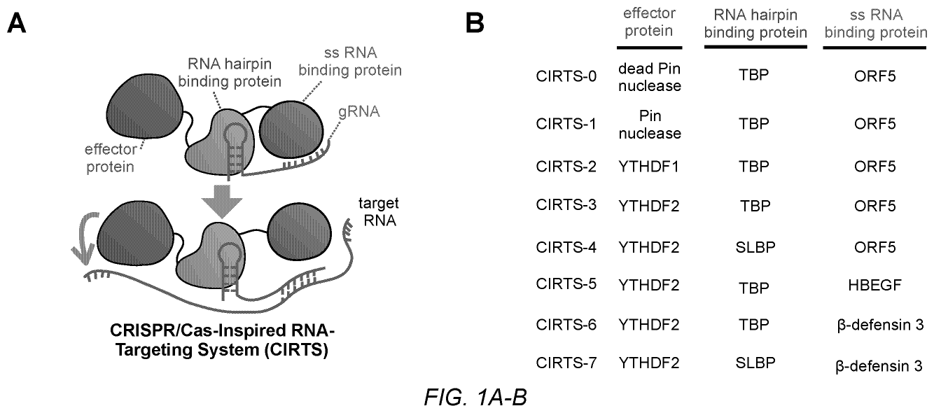

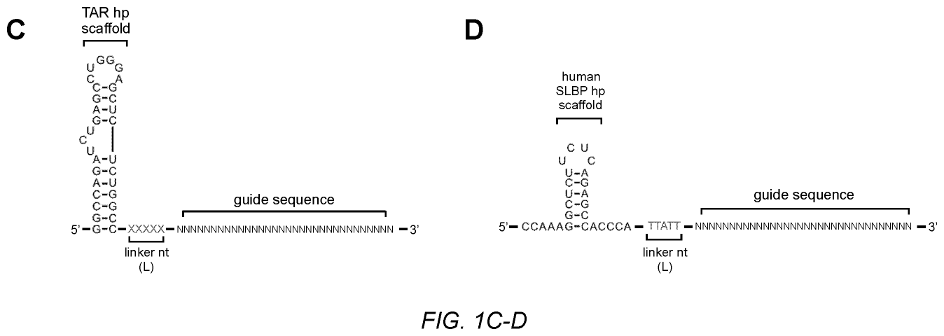

The inventors reasoned that a minimal programmable RNA-targeting system will need the following components: (1) an RNA hairpin-binding protein that serves as the core of the system and is a selective, high affinity binder to a specific RNA structure displayed on an engineered gRNA, (2) a gRNA that features both the structure that interacts with the engineered hairpin-binding protein and a sequence with complementarity to the target RNA of interest, and (3) an effector protein, such as a nuclease or epitranscriptomic regulator, that acts on the targeted RNA in a proximity-dependent manner. In some embodiments, a charged protein that binds to the displayed gRNA sequence non-specifically is used to stabilize and protect the guiding RNA prior to target engagement. (FIG. 1A). CIRTS combines multiple protein domains performing these functions in an engineered system.

Here, the inventors present the design and validation of CIRTS. First, the inventors engineered a programmable CIRTS ribonuclease, which they used for both in vitro and mammalian cell reporter assay optimization and validation. The inventors then demonstrate the versatility of CIRTS by showing that all four of the constituent parts, including the gRNA, the hairpin binding protein, the ssRNA binding protein, and the effector domain of CIRTS-1 can be substituted for other parts and developed five additional CIRTS (FIG. 1B).The inventors are able to target endogenous epitranscriptomic regulatory pathways, including degradation machinery and translational activation, using CIRTS engineered as programmable m⁶A reader proteins. Additionally, the inventors show that they can target endogenous transcripts for proximity-dependent nuclease-mediated decay, non-nuclease-mediated decay, or translation activation, using CIRTS. Finally, the inventors show they can target multiple genes simultaneously using orthogonal CIRTS. Taken together, this work validates the CIRTS strategy as a viable new approach to engineer RNA effector proteins that are small and assembled from human parts.

B. Results 1. Development and In Vitro Validation of CIRTS-1

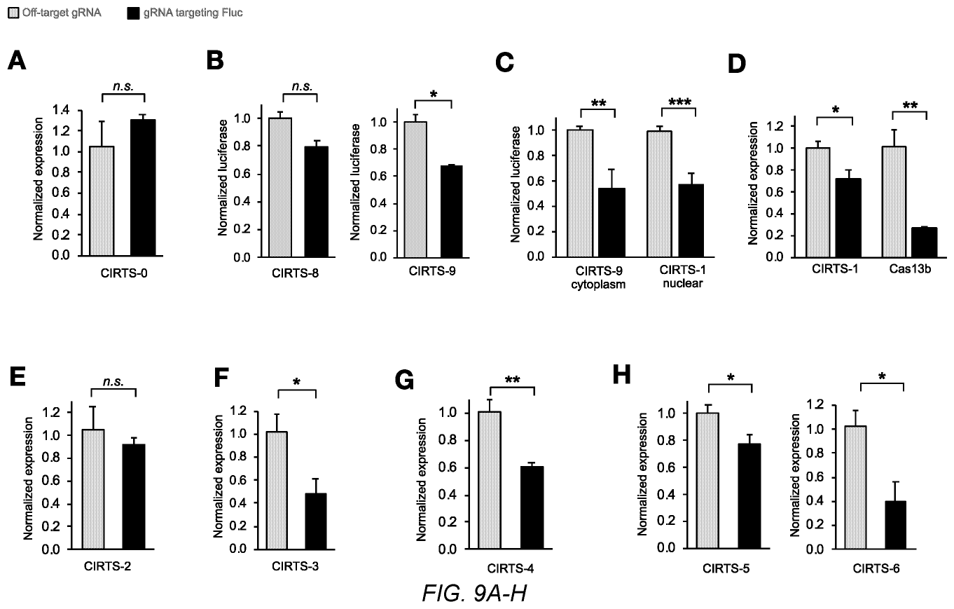

For the inventor's first-generation system, CIRTS-1, the inventors used an evolved version of the human hairpin-binding protein U1A protein (TBP6.7), which was previously engineered to bind the HIV TAR hairpin and has no endogenous human RNA hairpin targets (Blakeley and McNaughton, 2014; Crawford et al., 2016) (FIG. 1B). the inventors designed a gRNA that includes the TAR hairpin structure, a nucleotide linker sequence (L), and then a guiding sequence (FIG. 1C). To develop and validate the system, the inventors first engineered a programmable nuclease by fusing TBP6.7 to the Pin nuclease domain of human SMG6, which has been previously used as a non-specific proximity-dependent RNA endonuclease (Batra et al., 2017; Choudhury et al., 2012). Although this simplest design already displayed gRNA-mediated transcript degradation in cell-based assays (FIG. 9B, left), the performance was quite poor, which the inventors attributed to the potential degradation of the displayed targeting sequence. The protein surface and hairpin channel of Cas13 systems tend to be highly charged, likely to non-specifically bind and stabilize the guiding RNA sequence (Liu et al., 2017). To engineer this RNA protection function into the inventor's system, the inventors aimed to add in an RNA binding protein with non-specific, low affinity for single-stranded RNAs. However, the human protein toolbox did not readily contain an annotated small, non-specific single-stranded RNA binding protein. Therefore, the inventors developed CIRTS-1 using a small viral ssRNA binding protein, ORF5 (Zhou et al., 2006). Altogether, CIRTS-1 is composed of a protein fusion complex of ORFS-TBP6.7-Pin nuclease domain along with a corresponding gRNA (FIG. 1A).

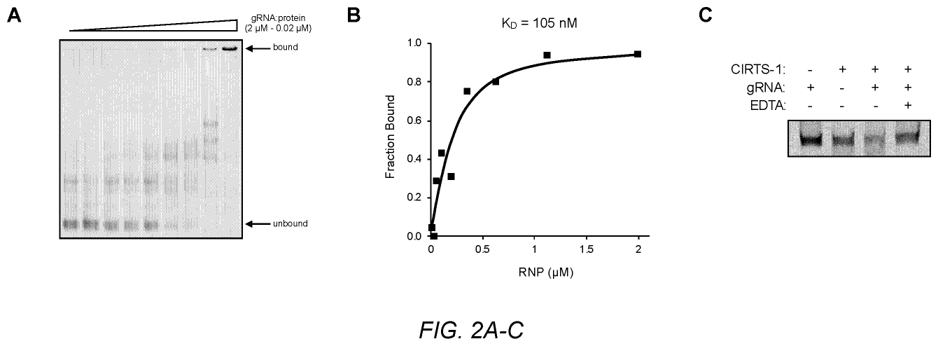

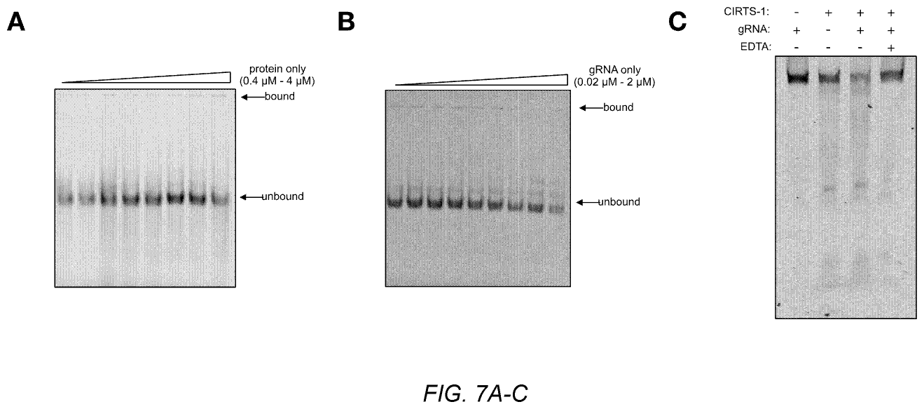

The inventors first characterized the programmable RNA binding and RNA nuclease activity of CIRTS-1 in vitro on model RNA target substrates. Using purified MBP-CIRTS-1 protein and gRNA in electrophoretic mobility shift assays (EMSA) run in the presence of EDTA to inactive the nuclease, the inventors found MBP-CIRTS-1 binds a target RNA in a gRNA-dependent manner with an apparent K_{D} of 105 nM (FIG. 2A and 2B). If either the gRNA (FIG. 7A) or CIRTS-1 protein (FIG. 7B) are omitted from the assay, no measurable binding is detected, confirming the gRNA-dependent binding mode as designed in the system. Moreover, in a cleavage assay, the inventors found that CIRTS-1 degrades an RNA substrate in a gRNA- and Mn²⁺-dependent manner (FIG. 2C). Collectively, these in vitro results validate the design principles behind CIRTS-1 and motivated the inventors to attempt to optimize the system for use in live cells.

2. Optimization of CIRTS-1 in Live Cells

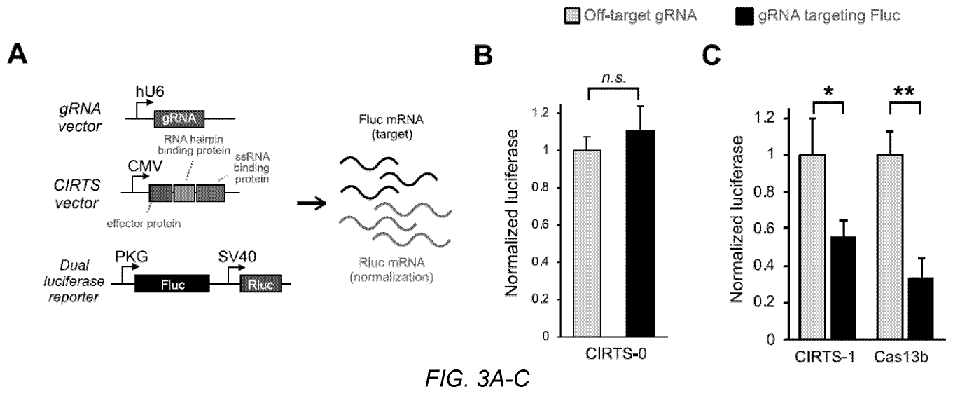

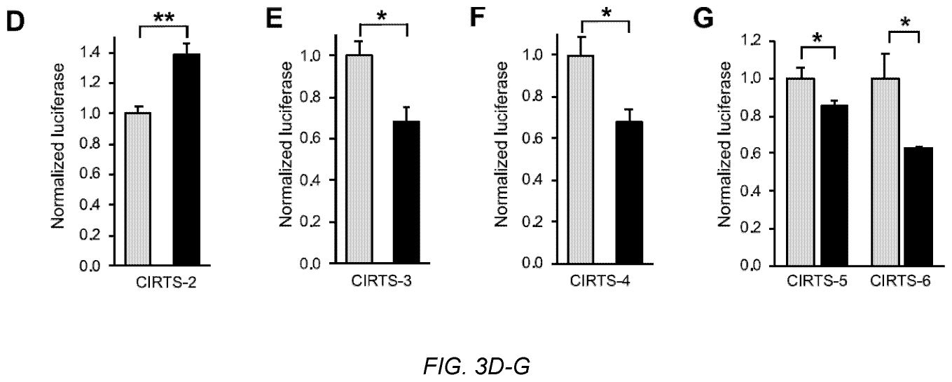

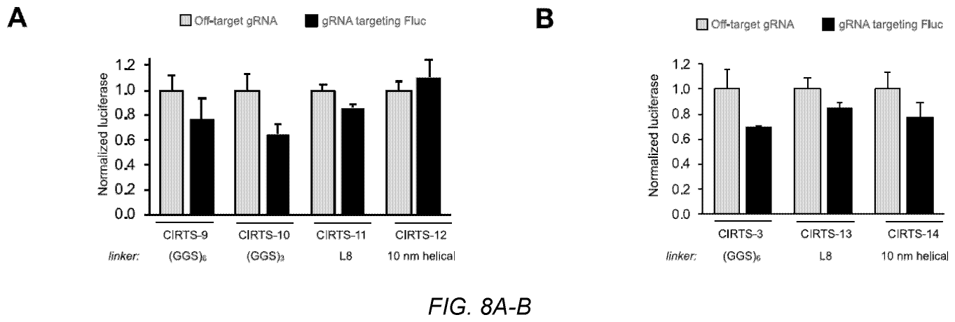

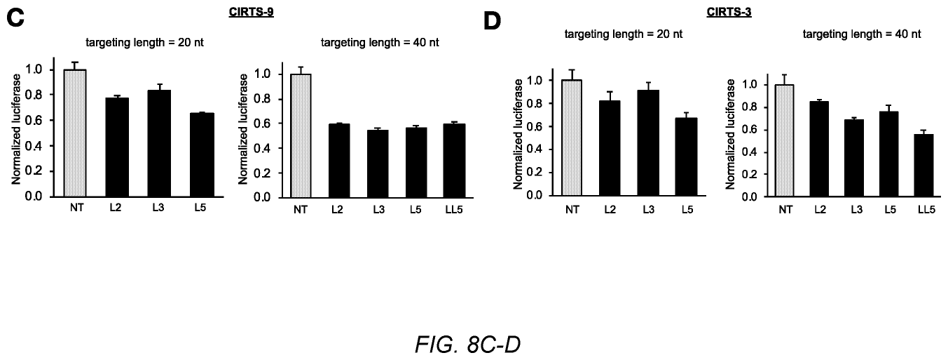

To test the target nuclease activity of CIRTS-1 in live mammalian cells, the inventors established a dual luciferase reporter assay that reports on gRNA-dependent transcriptional changes on a target RNA (FIG. 3A). Using this system, the inventors optimized the deployment of CIRTS-1 by assaying different protein linker types (FIG. 8A), gRNA structures (FIG. 8C), and CIRTS-1 cellular localization (FIG. 9C). After optimization, the inventors compared the ability of optimized CIRTS-1 system to degrade the target reporter RNA to the current state-of-the-art Cas13 system. The inventors designed gRNAs that target firefly luciferase mRNAs in the dual-luciferase reporter assay for both CIRTS-1 and Cas13b, as well as control, off-target gRNAs for each programmable nuclease. To test whether just binding to the transcript alters transcript levels, the inventors engineered CIRTS-0, which contains a previously reported ‘dead’ mutation in the nuclease domain of CIRTS-1 (Eberle et al., 2009), serving as a negative control. The inventors found that CIRTS-0 has no significant effect on the target transcript (FIG. 3B and FIG. 9A), indicating the CIRTS ribonucleoprotein target RNA complex is minimally perturbative to the RNA of interest. Next, the inventors tested whether CIRTS-1, with an active nuclease, could mediate degradation of the target. Indeed, the inventors found gRNA-dependent degradation of the target gene, measured at both the protein level as monitored by luciferase activity (FIG. 3C) and the mRNA levels as monitored by RT-qPCR (FIG. 9D). Encouragingly, the inventors found that CIRTS-1 is only slightly less efficient at targeting the luciferase reporter gene as compared to targeted nuclease degradation by Cas13b, indicating the CIRTS strategy is a viable method in live cells. Encouraged by the performance of CIRTS-1, the inventors next sought to assess the versatility of the design by testing whether the varied system components could be swapped for other parts and maintain CIRTS function.

3. Modularity of CIRTS

To explore the versatility of the CIRTS design, the inventors next tested different protein domains for each component of the CIRTS protein delivery system. First, the inventors assayed whether CIRTS could deliver RNA epitranscriptomic regulatory “reader” proteins, which they previously delivered using the dCas13b system (Rauch et al., 2018). The inventors swapped the Pin nuclease effector protein of CIRTS-1 for the N-terminal domain of YTHDF1, a cytoplasmic N⁶-methyladenosine (m⁶A) reader protein that recruits the translation machinery, to generate CIRTS-2. When CIRTS-2 is delivered to the same target sequence as the CIRTS-1 experiments, the RNA levels are relatively unchanged (FIG. 9E), but a significant increase in protein levels from the RNA are generated (FIG. 3D). The inventors then exchanged the YTHDF1 fragment for a fragment of YTHDF2, an m⁶A reader protein that recruits the RNA deadenylation machinery and induces RNA degradation, to generate CIRTS-3. Delivery of CIRTS-3 to the reporter mRNA induces degradation of the target transcript as measured by both RNA (FIG. 9F) and protein levels (FIG. 3E). CIRTS-1 through -3 demonstrate the versatility of the design strategy to deliver a range of effector protein cargoes to target RNA in live cells.

After demonstrating the modularity of the effector domain, the inventors set out to assess if other human parts could also be used for the RNA hairpin binding domain and non-specific ssRNA binding protein. The inventors replaced TBP6.7 in CIRTS-3 with the RNA hairpin binding domain of the human histone stem loop binding protein (SLBP) to generate CIRTS-4. Additionally, the inventors designed a gRNA based on the histone mRNA stem loop structure (FIG. 1D). Assaying CIRTS-4 in the reporter assay (FIG. 3F) and by RT-qPCR to assess RNA levels (FIG. 9G) revealed similar performance as CIRTS-3. Finally, the inventors sought to engineer entirely humanized versions of the CIRTS system. As stated earlier, the inventors designed the first proof-of-concept systems based on the viral non-specific, single-stranded RNA binding protein, ORF5. Although there are no annotated human single-stranded, non-specific RNA binding proteins, the inventors reasoned highly charged, cationic human proteins could fulfill the role of ORF5 in the CIRTS system (Cronican et al., 2011). The inventors therefore engineered HBEGF and β-defensin 3, two cationic human proteins, in the place of ORF5 in CIRTS-3 to generate CIRTS-5 and CIRTS-6, respectively. Again, deploying these programmable effectors in the luciferase reporter assay revealed gRNA-dependent degradation of the target gene (FIG. 3G). The inventors also assayed changes of RNA levels by qPCR and found a decrease for both CRITS-5 and CIRTS-6 (FIG. 9H). Collectively, the performance of CIRTS-1 through CIRTS-6 in the reporter assay demonstrates the modularity of the CIRTS design in all dimensions, including the hairpin-binding domain and corresponding gRNA, the single-stranded RNA binding protein, and the effector protein. Next, the inventors sought to test whether the CIRTS can also be used to target endogenous transcripts.

4. Targeting Endogenous mRNAs with CIRTS

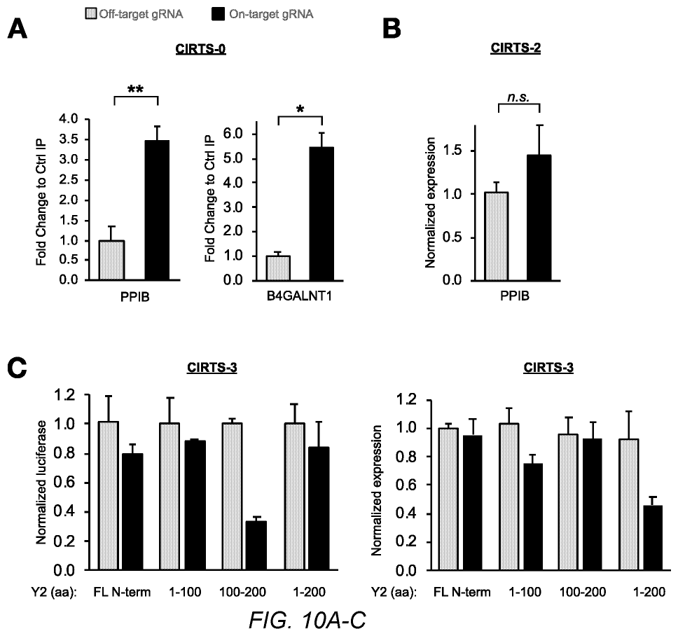

As a first step toward selectively targeting endogenous transcripts, the inventors verified that CIRTS-0 could bind a target endogenous transcript by analysis by RNA immunoprecipitation followed by RT-qPCR. The inventors designed gRNAs to target two endogenous transcripts that were previously targeted by Cas13 systems, PPIB, and SMARCA4 (Cox et al., 2017; Konermann et al., 2018). The inventors separately delivered each gRNA along with CIRTS-0 fused to a 3x FLAG-tag. The inventors then isolated total RNA, immunoprecipitated with an anti-Flag antibody, and quantified the relative amounts of each target RNA bound to the protein. Indeed, both endogenous transcripts were enriched between 2.5- and 5-fold in a gRNA-dependent manner (FIG. 10A), validating CIRTS can function as a programmable RNA-guided RNA binding protein on endogenous transcripts.

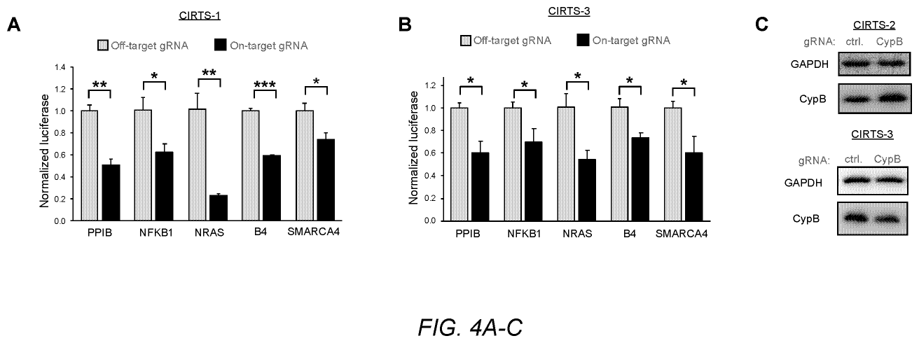

The inventors next sought to assess whether the CIRTS system could deliver an effector protein to a target endogenous transcript, using the CIRTS-1 programmable nuclease and CIRTS-3 programmable YTHDF2-mediated decay systems as exemplars. The inventors selected five RNA transcripts that have been previously validated as Cas13 targets, reasoning that these are accessible for RNA targeting by programmable RNA-binding systems. The inventors then designed gRNAs for each target, using the same binding sites on the targets that were previously used in Cas13 experiments, postulating these sites would also be accessible to CIRTS targeting (Konermann et al., 2018). The inventors assayed the effects of the CIRT system on RNA levels of each target by RT-qPCR. When cells were transfected with either CIRTS-1 or CIRTS-3, along with a specific gRNA expressing vector, the inventors saw a significant decrease in RNA level by RT-qPCR for each of the five endogenous transcripts: PPIB, NFKB1, NRAS, B4GALTN1, and SMARCA4 (FIG. 4A and 4B). The relative knockdown efficiency varied for each gene, which is also observed in other RNA-targeting systems and is potentially mediated by accessibility or other regulatory pathways specific to each gene. None-the-less, these results confirm that CIRTS can target endogenous transcripts and mediate decay through either active nuclease activity on the target or by triggering endogenous epitranscriptomic regulatory pathways.

Next, the inventors sought to assess whether CIRTS-2 could trigger protein production of endogenous transcripts through a YTHDF1-mediated epitranscriptomic pathways. The inventors selected an abundant transcript (CypB, the protein product of PPIB) with a reported, reliable antibody for analysis of protein production by Western blotting. Indeed, cells transfected with CIRTS-2 and an on-target gRNA showed an increase in protein level (FIG. 4C) without a change in RNA levels (FIG. 10B), confirming the YTHDF1 effect on the transcript. As a control, the same experiment performed with CIRTS-3, which delivers YTHDF2, results in decreased protein levels, which correlates with the decrease in mRNA levels (FIG. 3B). Taken together, these experiments show that the CIRTS platform is functional on endogenous transcripts in a gRNA-dependent manner, and can actively degrade a target transcript, trigger degradation machinery to act on the target transcript, or to active translation and increased protein production from the target transcript. The versatility of the CIRTS inspired the inventors to deploy them simultaneously to target more than one transcript.

5. Multiplexed Targeting of Multiple Endogenous RNAs with CIRTs

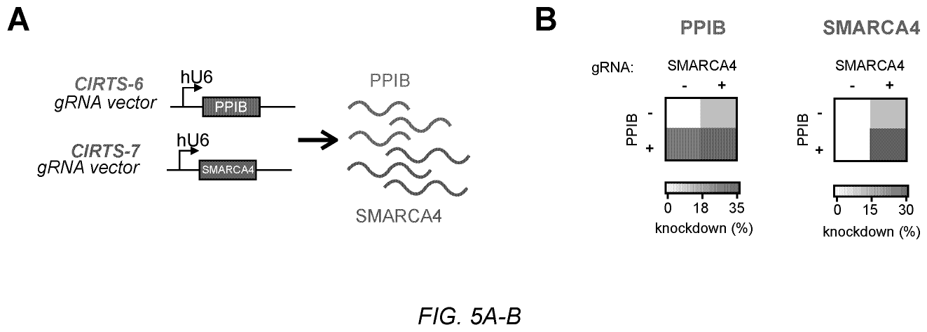

Finally, the inventors aimed to assess whether CIRTS engineered with different hairpin binding domains could functional orthogonally in live cells to selectively target different transcripts. In principle, the CIRTS built from the TBP hairpin binding domain and the CIRTS built from the SLBP hairpin binding domain, which each use separately engineered gRNAs (FIG. 1C and 1D), should be orthogonal to one another. For these orthogonality tests of multi-targeting CIRTS, the inventors deployed two fully-humanized CIRTS that each deliver YTHDF2, CIRTS-6 and CIRTS-7, which each bind different hairpin structures and should have minimal crosstalk between one another. The inventors aimed to use CIRTS-6 to target PPM and CIRTS-7 to target SMARCA4 (FIG. 5A). The inventors co-transfected cells with expression vectors for both CIRTS proteins, along with expression vectors for gRNAs for each CIRTS displaying either a control, non-targeted gRNA sequence, or a gRNA sequence targeting the respective endogenous gene. The inventors then assessed changes in relative RNA levels of the two target genes by RT-qPCR. Indeed, each CIRTS system degraded the target transcript in an on-target gRNA-dependent manner, without any crosstalk between the two systems (FIG. 5B). Critically, the inventors were able to selectively degrade one transcript or the other in the presence of both CIRTS proteins and were also able to simultaneously degrade both transcripts.

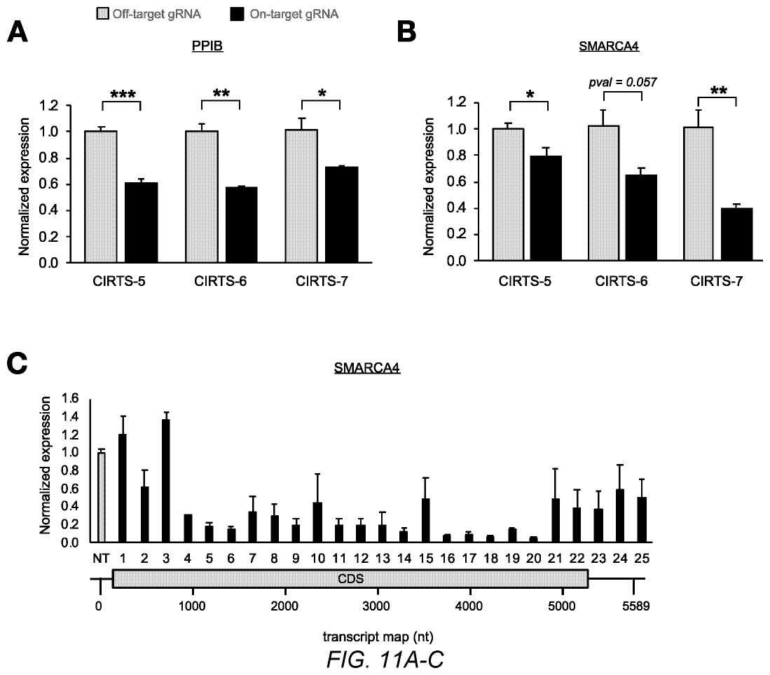

RNA degradation of CIRTS-6 showed levels comparable to when only CIRTS-6 is delivered to cells (FIG. 11A), targeting with CIRTS-7, however, resulted in slightly reduced RNA degradation as compared with individual transfection (FIG. 11B). For the multiplexed targeting experiment, the inventors transfected cells sequentially on two consecutive days with a CIRTS and a gRNA vector each day. The inventors attribute this decreased activity to decreased transfection efficiency on the second day of transfection with CIRTS-7. Additionally, while the TBP6.7-based CIRTS have RNA hairpins orthogonal to the endogenous mammalian cell machinery, the SLBP-based constructs could theoretically still bind to cellular histone mRNA, which could lead to a decrease in overall activity. Intriguingly, when both CIRTS were delivered with on-target gRNA, they performed at their best in this assay. At this point, the inventors conclude that the two orthogonal CIRTS systems can each be orthogonal to one another and can each simultaneously target endogenous transcripts in a gRNA-dependent manner. the inventors are currently working on engineering orthogonal CIRTS that have no more endogenous binding partners and can act without potentially perturbing cellular events.

C. Discussion

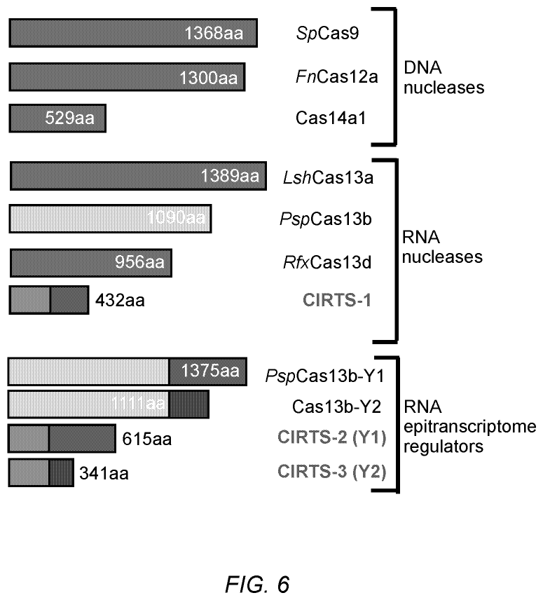

In summary, here the inventors presented CIRTS, a new strategy for engineering programmable RNA effector proteins. CIRTS are small, can be fully humanized, can target endogenous RNAs in live cells, and can work orthogonally and synergistically together for multidimensional control. As research tools, CIRTS should provide advantages to previous methods because of their smaller size. For example, CIRTS-2 and CIRTS-3 are 65 and 36 kDa respectively, while the comparable Cas13b-based programmable YTHDF1 and YTHDF2 systems are 155 and 126 kDa, respectively (FIG. 6). CIRTS-1 is even smaller than the smallest DNA-targeting Cas protein found to date, Cas14a (Harrington et al., 2018). The smaller size of the delivery system should be less perturbative to the endogenous transcript under study presenting opportunities for uncovering the roles of RNA regulatory proteins in live cells.

From a translational perspective, the CIRTS should offer several key advantages and opportunities. The humanized nature of the CIRTS will provide a pathway toward avoiding immune responses, opening up the potential for continuously-delivered therapies. With respect to using accessory proteins such as human β-defensin 3, this protein has been extensively studied and the structural bases for its function has been elucidated (Dhople et al., 2006; Kluver et al., 2005). This knowledge will enable one skilled in the art to engineer a β-defensin 3 peptide that retains its charged nature but abolishes its endogenous functions. Currently, the small size of the CIRTS will still allow for multiple regulatory proteins to be simultaneously delivered in a viral delivery system, for example to target one transcript for degradation and another for translational activation. The multiplexable capacity of the CIRTS couple with the small size and diversity of effector proteins that can be delivered opens up possibilities for cell reprogramming by targeting multiple genes at once in multiple dimensions.

From a broader perspective, the CIRTS platform demonstrates the potential of combining parts contained within the human protein toolbox to engineer proteins with new properties. The CIRTS system provides a new approach for studying and exploiting RNA regulation and will open up many future opportunities to intervene in cell regulation for disease treatment.

### Example 2

### Programmable RNA-Guided RNA Effector Proteins Built from Human Parts

A. Results

1. Design of CIRTS

While DNA-targeting Cas9-based systems employ complex biophysical mechanisms to unwind DNA and anneal to a target sequence (Rutkauskas et al., 2015; Sternberg et al., 2014; Szczelkun et al., 2014), mechanistic studies of Cas13 showed that RNA targeting is initiated by a central seed region in the gRNA (Liu et al., 2017). Additionally, Cas13 systems display substantial variability in sequence context targetability on individual transcripts (Abudayyeh et al., 2017; Cox et al., 2017; Konermann et al., 2018). Together these findings suggest that sequence complementarity between the gRNA and targeted transcript, as well as the accessibility of a given site, are key requirements for RNA targeting. The inventors sought to engineer a Cas13-inspired system that uses a defined protein-RNA interaction to display a gRNA sequence to deliver protein cargoes to a target RNA, similar to previous RNA tethering assays with overexpressed reporter constructs (Coller and Wickens, 2007). Indeed, hairpin-binding proteins and covalent RNA fusions have been used to deliver RNA editing machinery to transcripts (Montiel-Gonzalez et al., 2016; Sinnamon et al., 2017; Vogel et al., 2018).

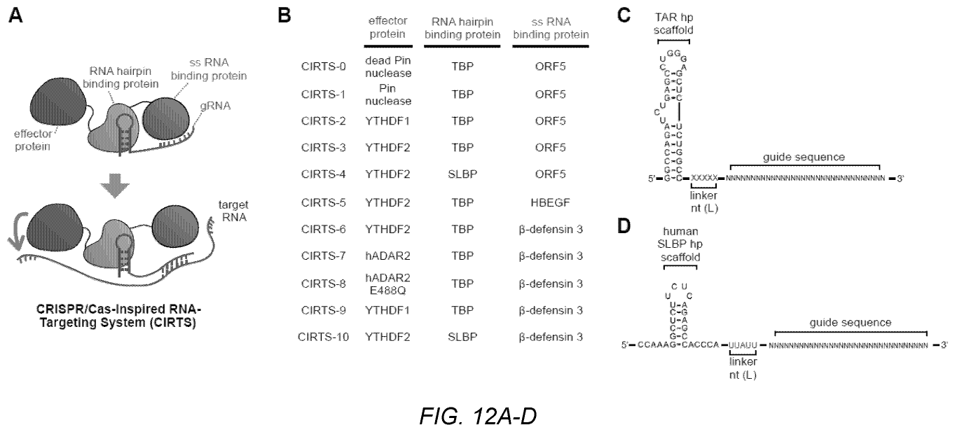

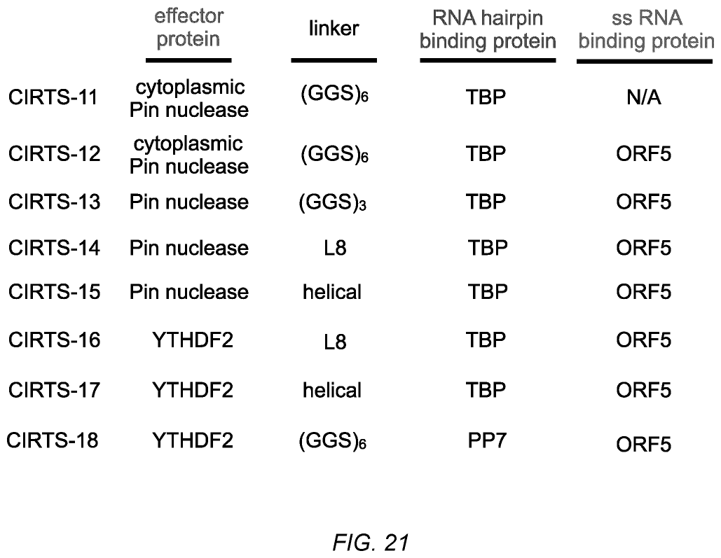

Based on the current characterization of Cas13 (Abudayyeh et al., 2017; Cox et al., 2017; Gootenberg et al., 2018; Konermann et al., 2018; Liu et al., 2017), the inventors reasoned that a minimal programmable RNA-targeting system may have four components: (1) an RNA hairpin-binding protein that serves as the core of the system and is a selective, high affinity binder to a specific RNA structure displayed on an engineered gRNA, (2) a gRNA that features both the structure that interacts with the engineered hairpin-binding protein and a sequence with complementarity to the target RNA of interest, (3) a charged protein that could bind to the displayed gRNA sequence non-specifically to stabilize and protect the guiding RNA prior to target engagement, and (4) an effector protein, such as a ribonuclease or epitranscriptomic regulator, that acts on the targeted RNA in a proximity-dependent manner (FIG. 12A). While Cas13 houses all of these functional components in a single protein domain (Liu et al., 2017; Tambe et al., 2018), the inventors envisioned engineering a system that combines multiple protein domains that each perform one of these functions, which the inventors termed CRISPR/Cas-inspired RNA targeting system (CIRTS). CIRTS vary in their module composition and are uniquely numbered as listed in FIG. 12A and FIG. 21.

2. Development and in Vitro Validation of CIRTS-1

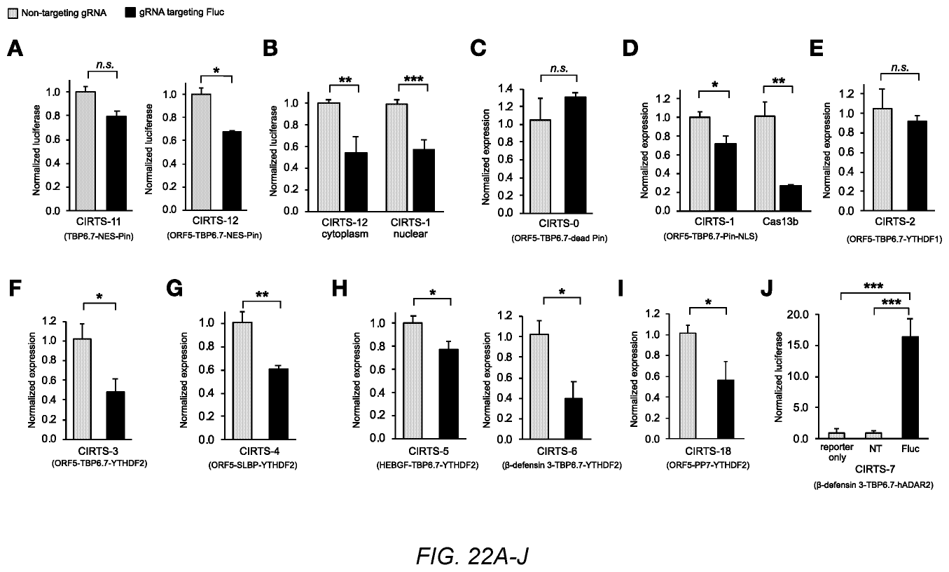

For the first-generation system, CIRTS-1, the inventors used an evolved version of the human hairpin-binding protein U1A protein (TBP6.7), which was previously engineered to bind the HIV trans-activation response (TAR) hairpin and has no endogenous human RNA hairpin targets (Blakeley and McNaughton, 2014; Crawford et al., 2016) (FIG. 12B). The inventors designed a gRNA that includes the TAR hairpin, a nucleotide linker sequence (L), and then a guiding sequence (FIG. 12C). To develop and validate the system, the inventors first engineered a programmable ribonuclease by fusing TBP6.7 to the Pin nuclease domain of human nonsense-mediated mRNA decay factor SMG6, which has been previously used as a non-specific proximity-dependent RNA endonuclease (Batra et al., 2017; Choudhury et al., 2012). Although this simplest design already displayed promising gRNA-mediated transcript degradation in cell-based luciferase assays (FIG. 22A, left), the performance was quite poor, which the inventors attributed to the degradation of the displayed guiding sequence. The protein surface and hairpin channel of Cas13 systems tend to be highly charged, likely to non-specifically bind and stabilize the guiding RNA sequence (Liu et al., 2017). To engineer this RNA protection function into the system, the inventors aimed to add a non-specific, low affinity single-stranded RNA binding protein (ssRNA binding protein). However, the human proteome did not readily contain an annotated small, non-specific single-stranded RNA binding protein to the best of the knowledge of the inventors. Therefore, the inventors developed CIRTS-1 using a small viral ssRNA binding protein, ORF5 (Zhou et al., 2006). Altogether, CIRTS-1 is composed of a protein fusion complex of ORFS-TBP6.7-Pin nuclease domain along with a corresponding gRNA.

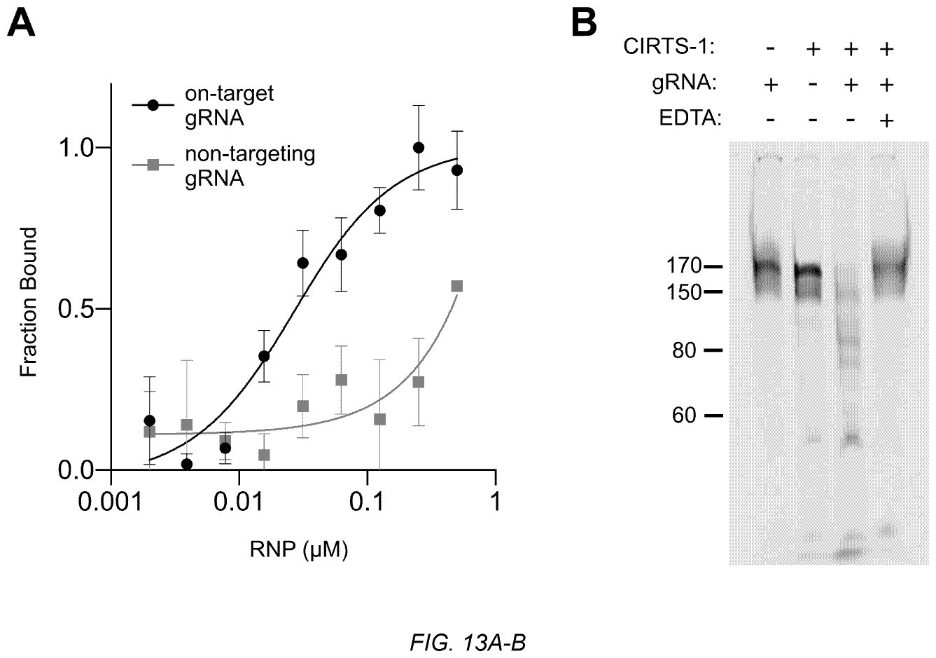

The inventors first characterized the programmable RNA binding and RNA nuclease activity of CIRTS-1 in vitro on model RNA target substrates. The Pin nuclease domain was previously shown to be active in the presence of Mn2+ and activity could be quenched by the addition of EDTA (Choudhury et al., 2012). Directly overexpressing CIRTS-1 led to insoluble protein in the cell pellet, which the inventors resolved by fusing an N-terminal MBP tag to CIRTS-1 (MBP-CIRTS-1). Using purified MBP-CIRTS-1 protein and gRNA in filter binding assays, the inventors found MBP-CIRTS-1 binds a target RNA in a gRNA-dependent manner with an apparent binding dissociation constant (KD) of 22 nM (FIG. 13A). Critically, if the inventors provide the system with a non-targeting gRNA, the inventors see ˜50-fold weaker binding. Moreover, in a cleavage assay, the inventors found that MBP-CIRTS-1 degrades an RNA substrate in a gRNA- and Mn2+-dependent manner, confirming the activity of the Pin ribonuclease domain in the fusion context (FIG. 13B). Collectively, these in vitro results validate the design principles behind CIRTS-1 and motivated the inventors to optimize the system for use in live cells.

3. Optimization of CIRTS-1 in Live Cells

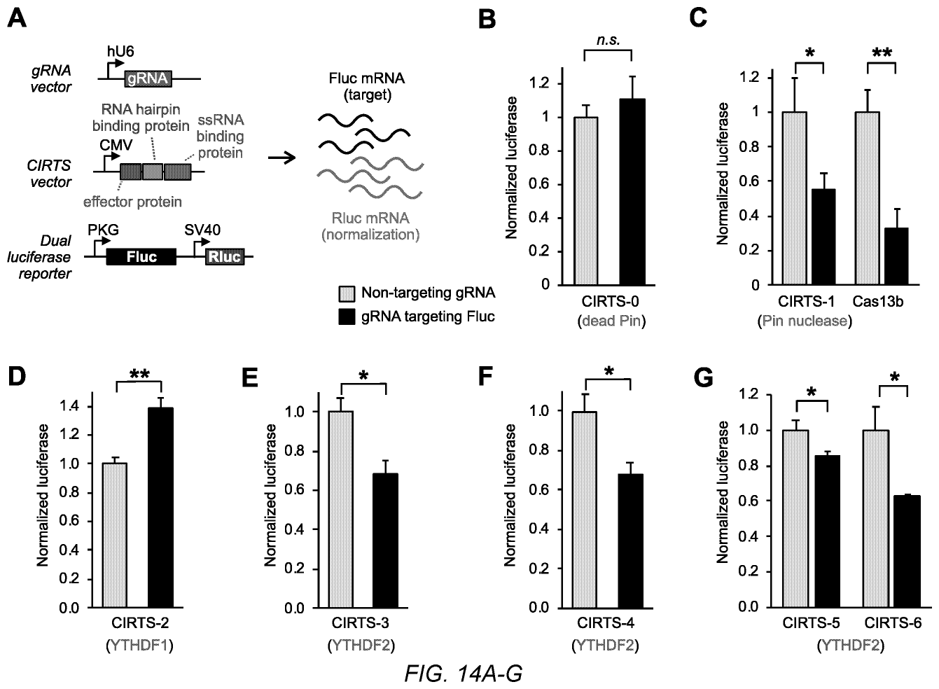

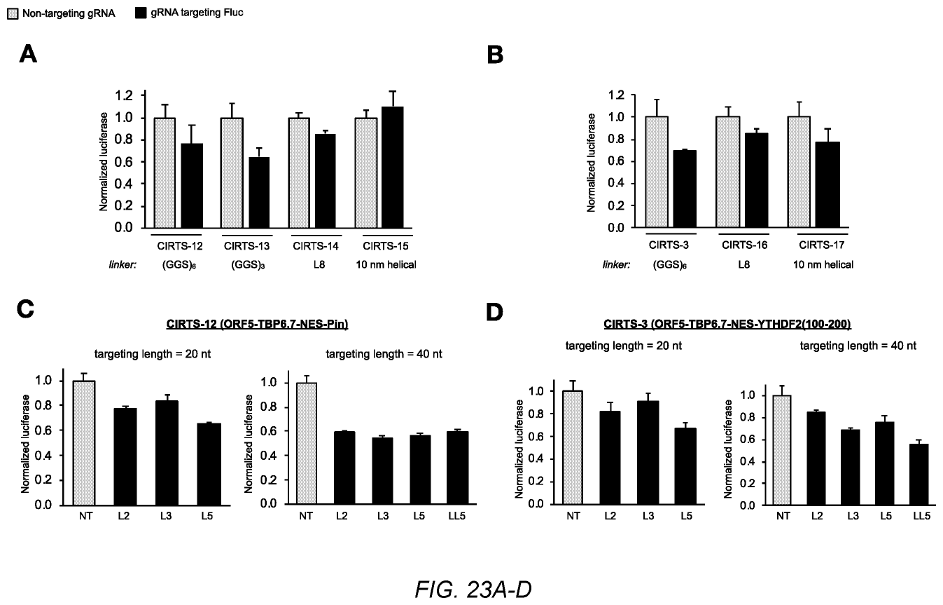

To test the target nuclease activity of CIRTS-1 in live mammalian cells, the inventors established a dual luciferase reporter assay that reports on gRNA-dependent transcriptional changes on a target firefly luciferase (Flue) RNA (FIG. 14A). Using this system, the inventors optimized the deployment of the Pin nuclease CIRTS by assaying different protein linker types (FIG. 23A), gRNA structures and lengths (FIG. 23C), and CIRTS nuclease cellular localization (FIG. 22B). The inventors found that a long flexible linker between the hairpin-binding protein and 40 nucleotide long gRNA resulted in the best knockdown efficiency. After optimization, the inventors compared the ability of optimized CIRTS-1 (Pin nuclease) system to degrade the target reporter RNA to the Cas13b system (Cox et al., 2017). The inventors designed gRNAs that target the firefly luciferase mRNA in the dual-luciferase reporter assay for both CIRTS-1 and Cas13b, as well as control, non-targeting gRNAs (targeting a lambda phage sequence) for each programmable nuclease. To test whether binding to the transcript alters protein levels, the inventors engineered CIRTS-0, which contains a previously reported ‘dead’ mutation in the Pin nuclease domain of CIRTS-1 (Eberle et al., 2009), serving as a negative control. The inventors found that CIRTS-0 has no significant effect on the expression level of the target transcript (FIG. 14B and FIG. 22C), indicating binding by the CIRTS ribonucleoprotein to a target RNA is minimally perturbative to the targeted transcript. Next, the inventors tested whether CIRTS-1, containing an active nuclease, could mediate degradation of the target. Indeed, the inventors found gRNA-dependent degradation of the target Fluc mRNA, measured at both the protein level as monitored by luciferase activity (FIG. 14C) and the mRNA levels as monitored by RT-qPCR (FIG. 22D). Both results suggest the CIRTS strategy is a viable method in live cells. Although CIRTS-1 is less efficient at targeting the reporter gene as compared to Cas13b, the performance was not dramatically different, especially considering that Cas13b systems have evolved to perform this knockdown function. Encouraged by the performance of CIRTS-1 (Pin nuclease), the inventors next sought to assess the versatility of the design by testing whether each component of the system, including the gRNA, hairpin binding domain, non-specific RNA binding domain, and effector protein, could be swapped for other parts to achieve CIRTS with diverse functions.

4. Modularity of CIRTS on Reporter Transcripts

To explore the versatility of the CIRTS design, the inventors next tested different protein domains for each component of the CIRT protein delivery system. First, the inventors assayed whether CIRTS could deliver RNA epitranscriptomic regulatory “reader” proteins, which the inventors previously delivered using the dCas13b system (Rauch et al., 2018). For the study, the inventors chose to focus on regulatory proteins of N6-methyladenosine, the most prevalent mRNA modification. On average each transcript contains three modifications sites with high m6A abundance detected in the 3′UTR, and m6A has been shown to have regulatory roles in splicing (Xiao et al., 2016), translation (Meyer et al., 2015; Wang et al., 2015), and stability (Du et al., 2016; Wang et al., 2013). The inventors exchanged the Pin nuclease effector protein of CIRTS-1 for the N-terminal domain of the YT521-B homology domain family protein 1 (YTHDF1), a cytoplasmic m6A reader protein that recruits the translation machinery (Wang et al., 2015), to generate CIRTS-2. Note that the inventors' current design does not include the C-terminal YTH domain of YTHDF1 that recognizes m6A to simplify studies of reader proteins in an m6A-independent manner and to avoid complications from varying m6A levels in cellular mRNAs when validating CIRTS. When CIRTS-2 (YTHDF1) is delivered to the same target sequence as the CIRTS-1 experiments, the RNA levels are relatively unchanged FIG. 22E), but a significant increase in protein levels from the RNA is generated (FIG. 14D), as expected based on the known translation-activating role of YTHDF1 (Wang et al., 2015). The inventors then exchanged the YTHDF1 fragment for a fragment of YTHDF2, a different m6A reader protein that recruits the RNA deadenylation machinery and induces RNA degradation (Du et al., 2016; Wang et al., 2013), to generate CIRTS-3. Delivery of CIRTS-3 (YTHDF2) to the reporter mRNA induces degradation of the target transcript as measured by both RNA (FIG. 22F) and protein levels (FIG. 14E). CIRTS-1 through -3 demonstrate the versatility of the design strategy to deliver a range of effector protein cargoes to target RNA in live cells.

After demonstrating the modularity of the effector domain, the inventors set out to assess if other human parts could also be used for the RNA hairpin binding domain and non-specific ssRNA binding protein. The inventors replaced TBP6.7 in CIRTS-3 (YTHDF2) with the RNA hairpin binding domain of the human histone stem loop binding protein (SLBP) to generate CIRTS-4 (YTHDF2). Concurrently, the inventors designed a gRNA based on the histone mRNA stem loop structure (FIG. 12D). Assaying CIRTS-4 (YTHDF2) in the reporter assay (FIG. 14F) and by RT-qPCR to assess RNA levels (FIG. 22G) revealed similar performance as CIRTS-3, confirming other hairpin binding domains can be used as the core of the CIRTS.

Next, the inventors sought to engineer entirely humanized versions of the CIRTS system. As stated earlier, the inventors designed the initial proof-of-concept systems based on the viral non-specific, single-stranded RNA binding protein, ORF5. Although there are no annotated human single-stranded, non-specific RNA binding proteins, the inventors reasoned highly charged, cationic human proteins could fulfill the role of ORF5 in the CIRTS system (Cronican et al., 2011). The inventors therefore engineered HBEGF and β-defensin 3, two cationic human proteins, in the place of ORF5 in CIRTS-3 to generate CIRTS-5 and CIRTS-6, respectively. Again, deploying these programmable effectors in the luciferase reporter assay revealed gRNA-dependent degradation of the target gene (FIG. 14G and S2H) mediated by the YTHDF2 epitranscriptomic regulation.

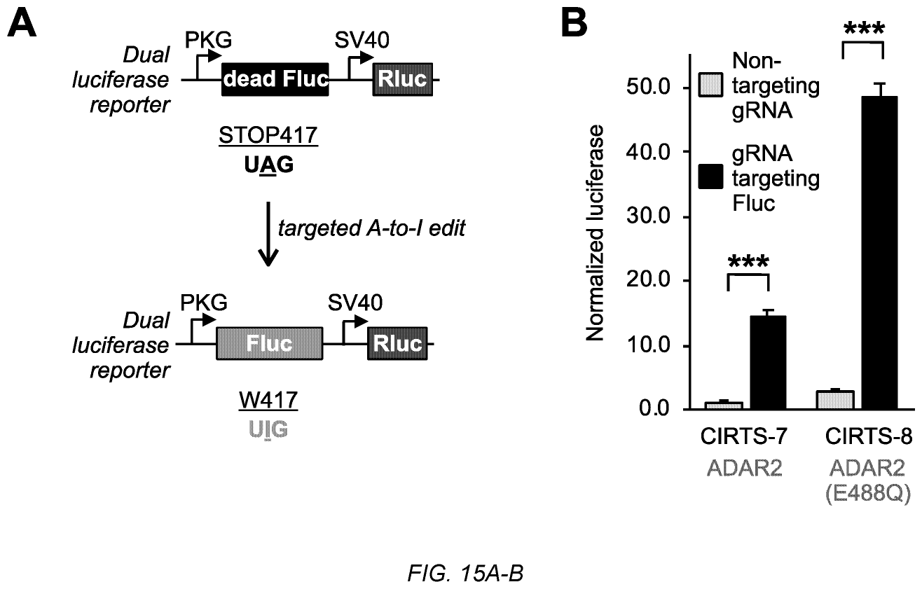

Finally, the inventors used CIRTS to deliver the catalytic domain of human ADAR2 (hADAR2) to RNA transcripts to confirm CIRTS' versatility in scope of functions with an additional effector protein. The inventors designed a dual luciferase reporter that contains a G-to-A mutation in the coding region of firefly luciferase resulting in a premature stop of translation and no measurable firefly luciferase activity (FIG. 15A and 22J). The inventors then deployed CIRTS to deliver wt ADAR2 (CIRTS-7) or hADAR 2 E488Q (CIRTS-8), a known hyperactive mutant of hADAR2 (Kuttan and Bass, 2012), to the mutated position, which resulted in gRNA-dependent rescue of luciferase activity in both cases (FIG. 15B). The hyperactive hADAR2 mutant showed higher editing efficiency and a higher background in the absence of an on-target gRNA based on luciferase assay. However, using the hyperactive mutant could be beneficial to allow targeting of a wider substrate scope as it has relaxed sequence constraints (Kuttan and Bass, 2012). Collectively, the performance of these various CIRTS in the reporter assays demonstrates the modularity of the CIRTS design, including the hairpin-binding domain and corresponding gRNA, the single-stranded RNA binding protein, and the effector protein.

5. Targeting Endogenous mRNAs with CIRTS

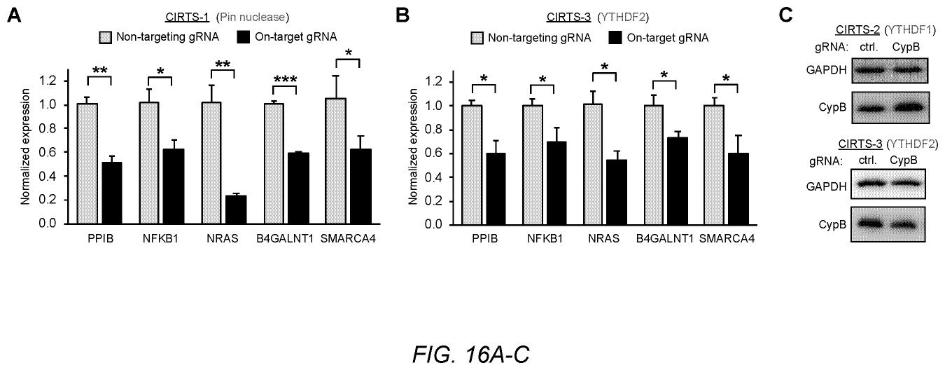

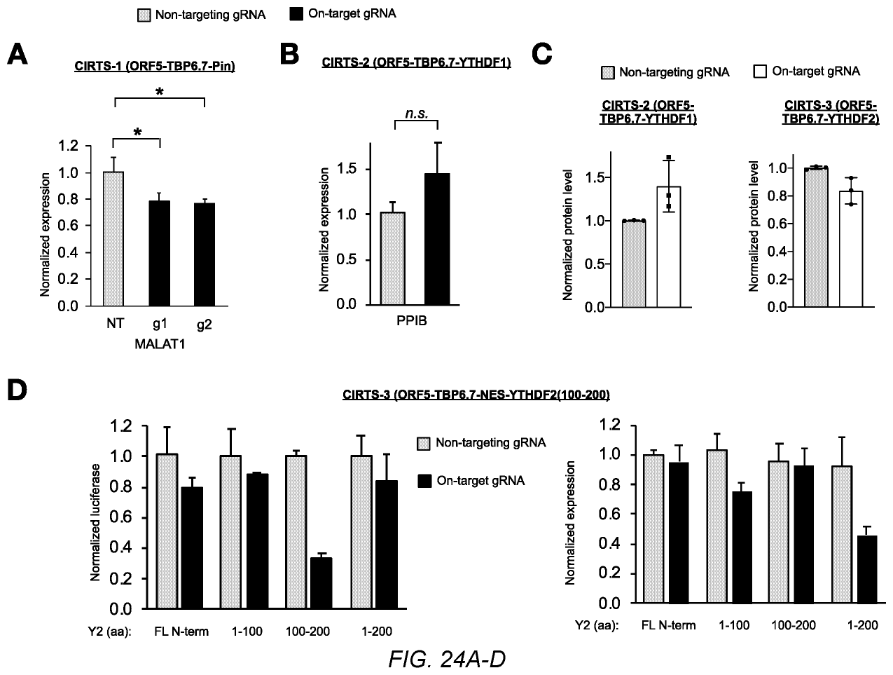

The inventors next sought to assess whether the CIRTS system could deliver an effector protein to a target endogenous transcript, using the CIRTS-1 programmable nuclease and CIRTS-3 programmable YTHDF2-mediated decay systems as exemplars. The inventors selected five RNA transcripts that have been previously validated as Cas13 targets, reasoning that these are accessible for RNA targeting by programmable RNA-binding systems. The inventors then designed gRNAs for each target, using the same binding sites on the targets that were previously used in Cas13 experiments (Abudayyeh et al., 2017; Konermann et al., 2018). The inventors assayed the effects of the CIRT system on RNA levels of each target by RT-qPCR. When cells were transfected with either CIRTS-1 or CIRTS-3, along with a specific gRNA expressing vector, the inventors observed a significant decrease in RNA level by RT-qPCR for each of the five endogenous mRNA transcripts: PPIB, NFKB1, NRAS, B4GALNT1, and SMARCA4 (FIG. 16A and 16B). In addition to targeting mRNA, the inventors also verified that the inventors can target other RNA species such as lncRNA by targeting CIRTS-1 (Pin nuclease) to MALAT1 (FIG. 24A). The relative knockdown efficiency varied for each gene, which is also observed in other RNA-targeting systems and is potentially mediated by accessibility or other regulatory pathways specific to each gene. Nonetheless, these results confirm that CIRTS can target endogenous transcripts and mediate decay through either active nuclease activity on the target or by triggering endogenous epitranscriptomic regulatory pathways.

Next, the inventors set out to assess whether CIRTS-2 could trigger protein production of an endogenous transcript through a YTHDF1-mediated epitranscriptomic pathways. The inventors selected an abundant transcript PPIB with a reported, reliable antibody for analysis of CypB (the protein product of PPIB) protein production by Western blotting. Indeed, cells transfected with CIRTS-2 and an on-target gRNA showed an increase in protein level (FIG. 16C and FIG. 24C) without a change in RNA levels (FIG. 24B), consistent with prior reported YTHDF1 effects on the transcript (Wang et al., 2015). As a control, the same experiment performed with CIRTS-3, which delivers YTHDF2, results in slight decrease in protein levels, which correlates with the decrease in mRNA levels (FIG. 16B and 24C).

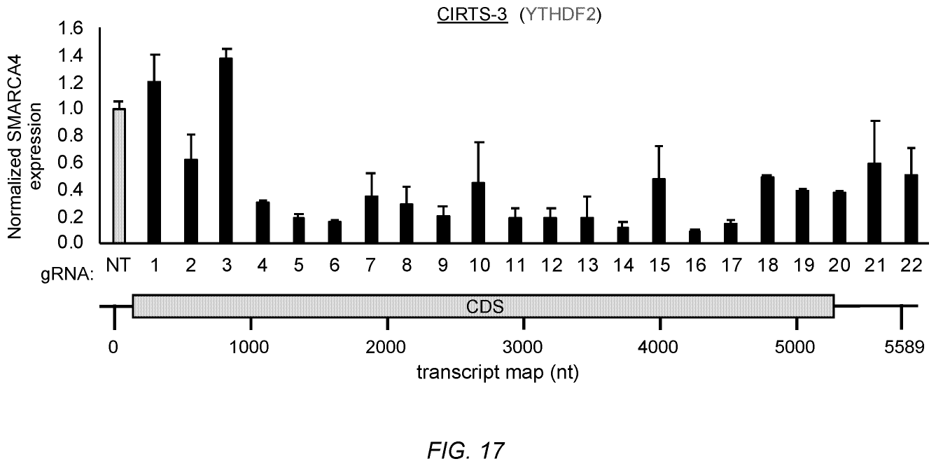

Finally, as a first test of transcript position-specific effects, the inventors tiled gRNAs along the SMARCA4 mRNA and tested YTHDF2-mediated decay by CIRTS-3. The inventors found dramatically different performance of the system depending on where the gRNA lands on the targeted mRNA (FIG. 17), which is likely the result of both CIRTS binding accessibility and the regulatory protein sequence requirements. Taken together, these experiments show that the CIRTS platform is functional on endogenous transcripts in a gRNA-dependent manner, and can actively degrade a target transcript, trigger degradation machinery to act on the target transcript, or activate translation and increase protein production from the target transcript.

6. Targeting Specificity of CIRTS

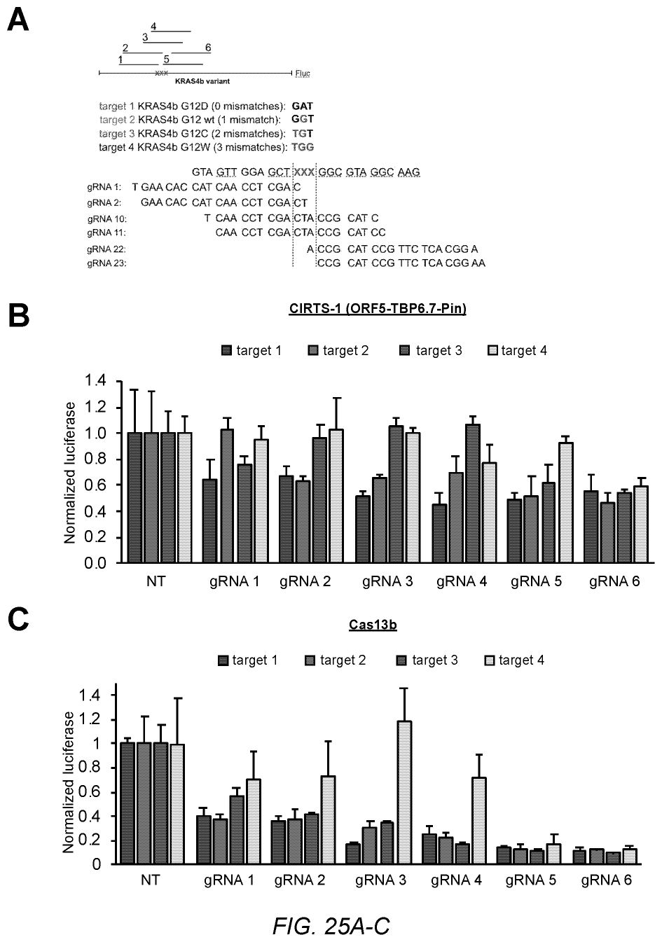

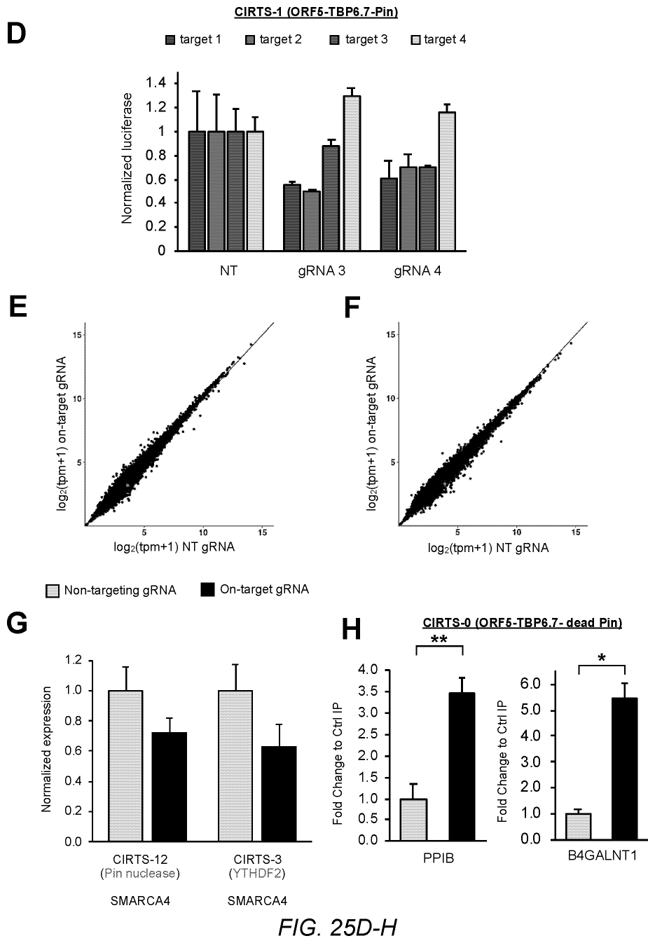

To gain insights into how specific CIRTS is at targeting RNA substrates, the inventors designed a series of experiments that address the sensitivity of the gRNA to mismatches, transcriptome-wide off-targets, and endogenous substrate targeting. To assess mismatch tolerance, the inventors designed a luciferase-based mismatch experiment that allows the inventors to assay targeting effects when introducing one, two, or three mismatches into the duplex formed between gRNA and target RNA. The inventors chose to fuse the disease-relevant KRAS4b transcript to the luciferase reporter and asked whether the engineered system can differentiate between the cancer-associated G12D (target 1), the wild type (target 2), the G12C (target 3), and a G12W (target 4) KRAS4b variants (FIG. 25A). The inventors found that CIRTS yields comparable knockdown of the G12D and wild type variants indicating that one mismatch does not cause large changes in targeting specificity (FIG. 25B). However, when the inventors targeted the system to the G12C and G12W reporters, which contain two and three mismatches respectively, CIRTS knockdown efficiency decreased.

The inventors next assessed whether increasing the gRNA length could affect the mismatch tolerance of CIRTS, focusing on mismatches in the center region of the duplex formed between gRNA and target RNA as they showed the largest effect on knockdown efficiency in the assay. As observed with the shorter 20 nt gRNA, the inventors see no difference in knockdown efficiency when the inventors target a reporter with no or one mismatches. However, a longer 40 nt gRNA can rescue some of the effects when the two-mismatch variant was targeted, indicating that the gRNA length contributes to the specificity of the system (FIG. 25D). As a comparison to existing technology, the inventors subjected Cas13b to the same mismatch assay, which showed that Cas13b is less sensitive to mismatches in general. Targeting Cas13b to reporters with one and two mismatches yielded little change in knockdown efficiency, while three mismatches led to a substantial decrease in knockdown efficiency (FIG. 25C). Both Cas13b and CIRTS are most sensitive to mismatched base-pairing in the center of the duplex formed between gRNA and target RNA, a finding that agrees well with previous studies of Cas13b (Abudayyeh et al., 2017).

To assess transcriptome-wide off-targets, the inventors subjected the system to RNA sequencing. The inventors assayed effects of the CIRTS Pin nuclease and CIRTS YTHDF2 targeting the endogenous transcript SMARCA4 (FIG. 25E and 25F). In both cases, the inventors find no statistically significant off-targets. However, while the inventors see knockdown of the targeted transcript and even statistically significant knockdown by CIRTS-3 (YTHDF2) when the inventors look at the target transcript only (pval <0.1), the knockdown levels do not fall into a statistically significant region when evaluated in a transcriptome-wide manner (qval <0.1). At this point the inventors conclude that overexpression of the system does not introduce large perturbations to the transcriptome, and taken together with the inventors' mismatch studies, indicates that the targeting specificity and therefore knockdown efficiency can be further optimized by further optimization in future studies.

To verify CIRTS bind the transcript of interest, the inventors furthermore performed RNA immunoprecipitation followed by RT-qPCR. The inventors designed gRNAs to target two endogenous transcripts that were previously targeted by Cas13 systems, PPIB, and B4GALNT1 (Cox et al., 2017; Konermann et al., 2018). The inventors separately delivered each gRNA along with CIRTS-0 (dead nuclease CIRTS) fused to a 3x FLAG-tag. The inventors then subjected lysates to immunoprecipitation with an anti-Flag antibody, and quantified the relative amounts of each target RNA bound to the protein. Indeed, both endogenous transcripts were enriched between 2.5- and 5-fold in a gRNA-dependent manner (FIG. 25H), confirming CIRTS function as a programmable RNA-guided RNA binding protein on endogenous transcripts.

7. Multiplexed targeting of multiple endogenous RNAs with CIRTS

Together the targeting specificity and the modularity of CIRTS inspired the inventors to extend the application of CIRTS in a multiplexed targeting manner. Rather than delivering a single effector protein and targeting a single transcript at a time, the inventors set out to test whether CIRTS can target more than one transcript, or deliver more than one effector protein in the same sample. In principle, CIRTS built from the TBP hairpin binding domain and CIRTS built from the SLBP hairpin binding domain, which each use separately engineered gRNAs (FIG. 12C and 12D), should be orthogonal to one another, permitting selective targeting of multiple transcripts with either the same or even different CIRTS.

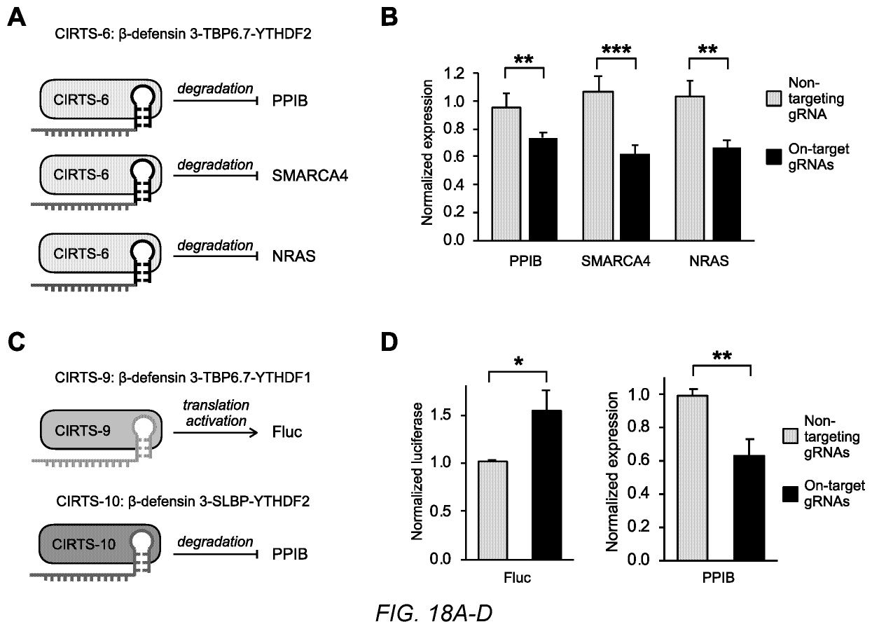

First, the inventors tested whether a single CIRTS can be used to simultaneously target multiple transcripts. The inventors co-transfected cells with CIRTS-6 along with three gRNAs targeting PPIB, SMARCA4, and NRAS and assessed changes in RNA level by RT-qPCR. As expected, the inventors observed a decrease in RNA levels for all three targeted transcripts (FIG. 18A and 18B). However, the inventors observed a slight decrease in efficiency when the inventors deploy several gRNAs or CIRTS in the same cells. The inventors attribute this decrease to simultaneous transfection of cells with four plasmids. Further optimization of the protein and gRNA levels and vectors are likely required to regain the performance of the single CIRTS/gRNA experiments.

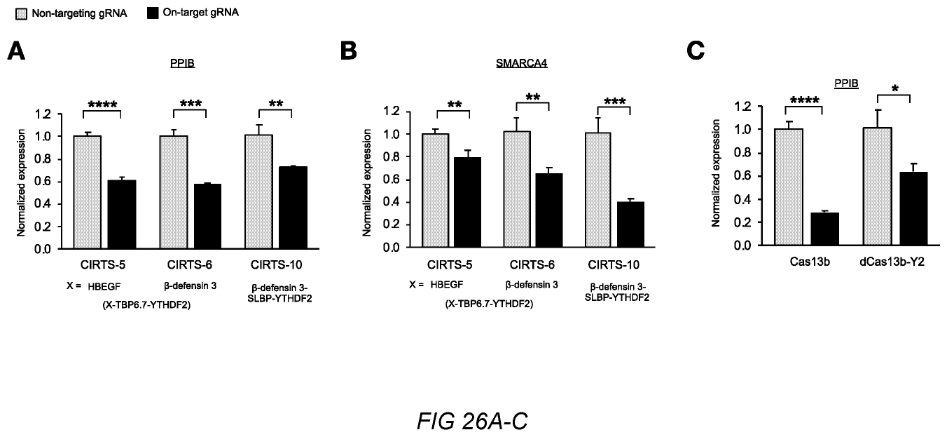

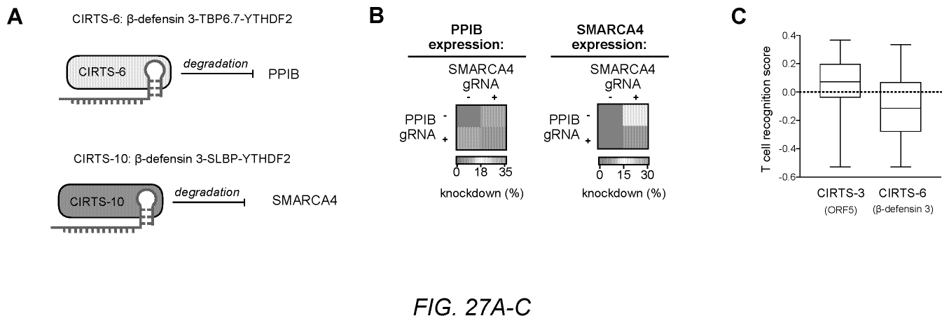

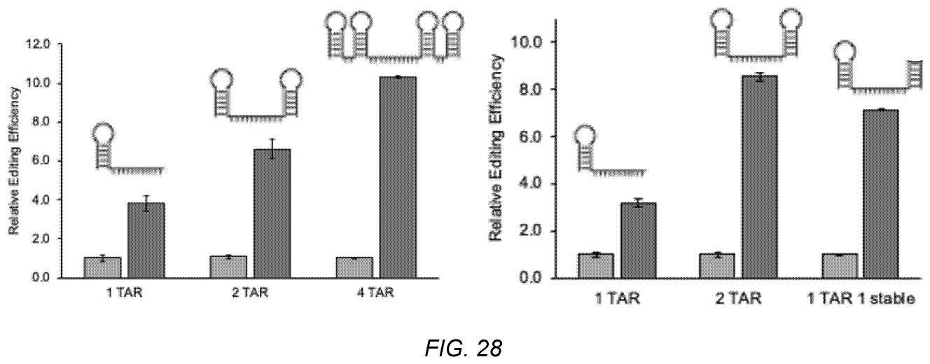

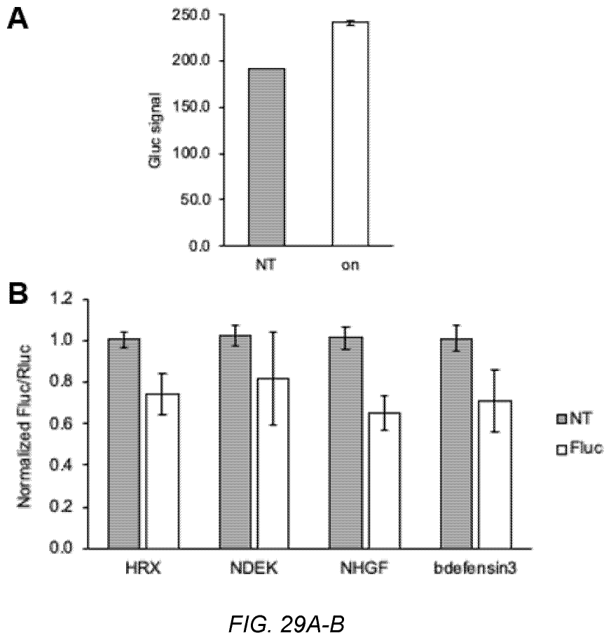

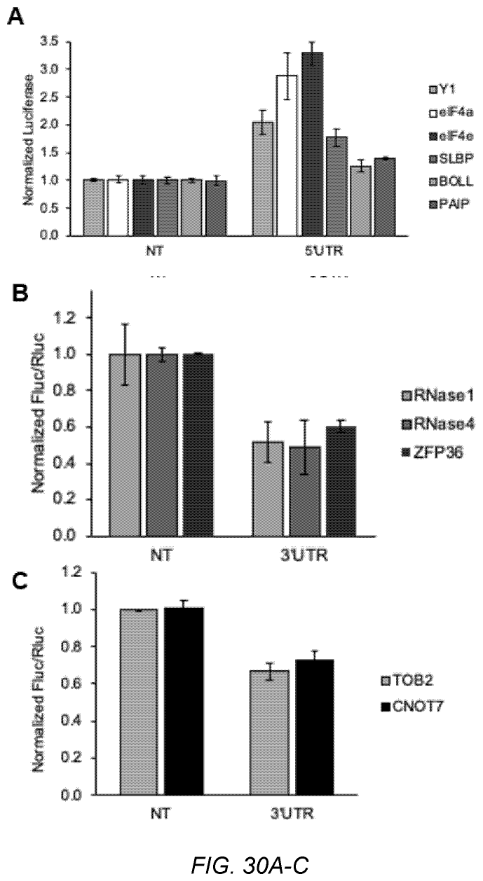

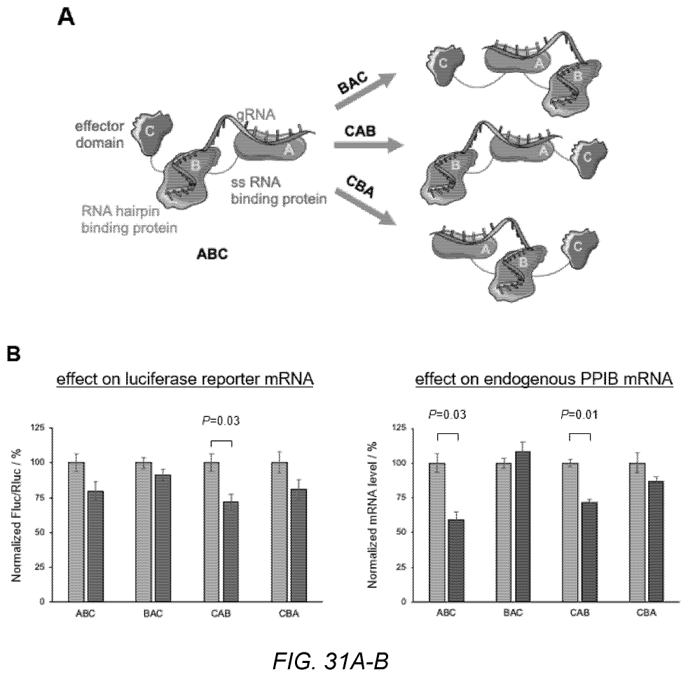

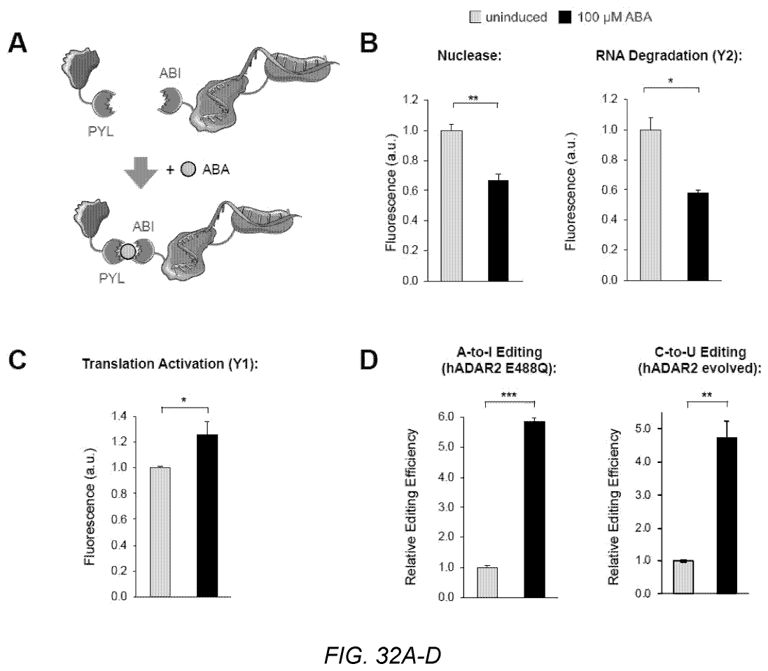

To test whether two different types of effectors can be used simultaneously, the inventors next deployed both CIRTS-9, a fully humanized version of the YTHDF1 construct, to target firefly luciferase and CIRTS-10 (YTHDF2) to target SMARCA4 (FIG. 18C and 18D). The inventors find that both proteins are active and induce the anticipated increase in luciferase protein and decrease in RNA levels respectively. Moreover, to further corroborate the orthogonality of multiple-targeting CIRTS, the inventors deployed two CIRTS, CIRTS-6 (TBP6.7) and CIRTS-10 (SLBP) (FIG. 26A and 26B), but use different hairpin-binding modules, to deliver YTHDF2 to two different endogenous target mRNAs (FIG. 27A). Each CIRT system degraded the target transcript in an on-target gRNA-dependent manner, with minimal crosstalk between the two systems (FIG. 27B).

At this point, the inventors conclude that the TBP6.7 and SLBP-based CIRTS can each simultaneously target endogenous transcripts in a gRNA-dependent manner. The inventors are currently working on engineering orthogonal CIRTS that have no endogenous binding partners by evolving human proteins toward new specificities, as was done with TBP6.7. Although not human-derived, the inventors found that other hairpin-binding systems, such as PP7, can also be used to generate CIRTS, suggesting it is possible to generate a range of selective and orthogonal systems (FIG. 22I). CIRTS allow for multiple regulatory proteins to be simultaneously delivered, for example to target one transcript for degradation and another for translational activation, opening up possibilities for cell reprogramming by targeting multiple genes at once in multiple dimensions (Bao et al., 2017; Gao et al., 2016).

8. Viral Delivery of CIRTS by AAV

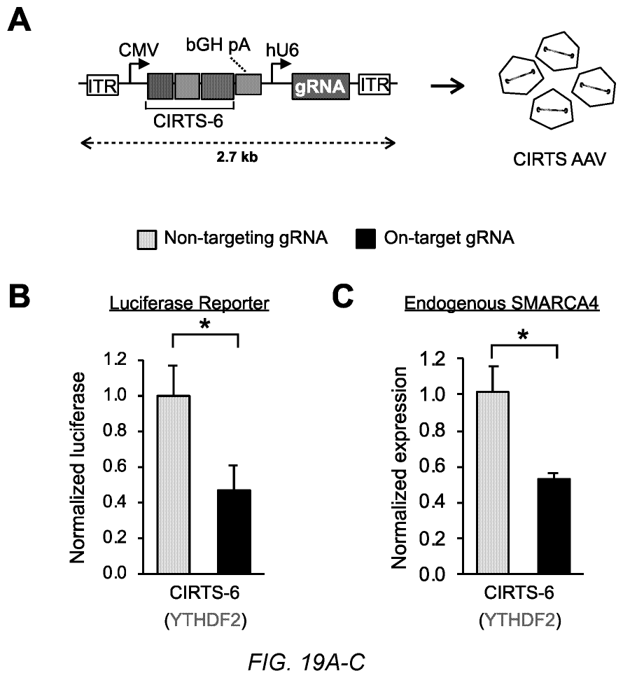

Aside from the human-derived nature of CIRTS, another core advantage is the small size of CIRTS, which should permit more efficient viral packaging and delivery. Adenovirus-associated virus (AAV) is a versatile delivery vehicle to deliver transgenes and gene therapies to different cell types due to wide range of serotypes available (Gao et al., 2005), low immune response stimulation (Vasileva and Jessberger, 2005), and low risk of genome insertion (Gao et al., 2005; Naso et al., 2017). However, it has been challenging to package and deliver many Cas13 proteins due to a limited packaging capacity of about 4.7 kb (Wu et al., 2010). To showcase the possibility of CIRTS to be delivered by AAV, the inventors designed a dual CIRTS-6/gRNA transfer plasmid and packaged it in the AAV delivery vehicle. The total insert, including the CIRTS protein and gRNA, is only 2.7 kb (FIG. 19A). The inventors found that transduction of HEK293T cells with the generated virus recapitulates the knockdown efficiency of CIRTS-6 on both the luciferase reporter as well as an endogenous target (FIG. 19B and 19C), confirming viral-delivered CIRTS are still functional and providing a pathway toward clinical deployment. In future applications, one could imagine packing more than one CIRT system into the AAV delivery vehicle to simultaneously target one transcript for upregulation and one transcript for degradation as previously shown by transient transfection.

B. Discussion

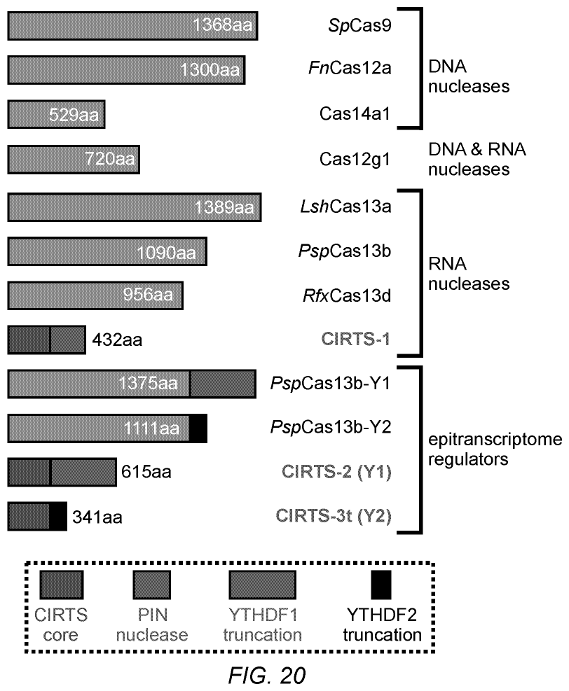

In summary, here the inventors presented CIRTS, a versatile strategy for engineering programmable RNA effector proteins. CIRTS are small, can be fully humanized, can target endogenous RNAs in live cells, and can work for multidimensional transcriptome control. As research tools, CIRTS should provide advantages to previous methods because of their smaller size. For example, CIRTS-2 and CIRTS-3 are 65 and 36 kDa respectively, while the comparable Cas13b-based programmable YTHDF1 and YTHDF2 systems are 155 and 126 kDa, respectively (FIG. 20). CIRTS-1 is even smaller than the smallest DNA-targeting Cas protein found to date, Cas14a and the smallest Cas12g RNA-targeting protein (Harrington et al., 2018; Yan et al., 2019).

From a translational perspective, CIRTS should offer several key advantages and opportunities. The humanized nature of the CIRTS will provide a pathway toward avoiding immune responses, opening up the potential for continuously-delivered therapies. While the fusions between the human proteins in the CIRTS present potential limitations in the design where the immune system could respond to (Glaesner et al., 2010), this is a problem that can in principle be engineered around. When the inventors computationally predicted the immunogenicity of the highest likelihood MHC I binding peptides in the inventors' engineered constructs, the inventors find that the fully humanized CIRTS shows lower propensity to cause immune reactions (FIG. 27C), but further experimental testing is needed to discover where the limitations in the design emerge.

Several known challenges remain with the current CIRT system. First, the alternative hairpin binding protein SLBP in its current form has an endogenous RNA hairpin binding partner, which could influence stem loop RNA trafficking. To minimize endogenous effects of the inventors' fusion constructs, the inventors only included the minimal RNA recognition motif (RRM) necessary for hairpin recognition in the system and omitted regions of potential interactions with other proteins or nucleic acids. Likewise, the inventors tried to keep the required RNA hairpin as small as possible to avoid potential endogenous interactions. The stem loop hairpin was already very short and could not be further truncated but the inventors chose to only use the minimally required region necessary for TBP6.7 binding to the TAR hairpin, resulting in a gRNA with less than half the original hairpin length. Second, the cationic peptide, β-defensin 3, in its current form can theoretically still interact with its intracellular binding partners and elicit unwanted biological responses. However, human β-defensin 3 has been extensively studied and the structural bases for its function has been elucidated (Dhople et al., 2006; Kluver et al., 2005). The inventors can leverage this knowledge as the basis to engineer β-defensin 3 mutants that retain the highly charged nature required for CIRTS, but abolish endogenous functions, in order to engineer a human part-based, orthogonal RNA targeting system.

From a broader perspective, the CIRTS platform demonstrates the potential of combining parts contained within the human protein toolbox to engineer proteins with new properties. The presented CIRTS were created through minimal protein engineering and optimization efforts, but function nearly as well as the naturally-evolved CRISPR/Cas systems. In particular, when the inventors compared the Cas13b-based knockdown by its endogenous nuclease and by delivering YTHDF2 (FIG. 26C) to CIRTS-mediated knockdown, the inventors find that while the Cas13b nuclease performs substantially better than CIRTS nuclease, the inventors see no difference in knockdown mediated by YTHDF2, suggesting CIRTS-3 (YTHDF2) in its current state can already be used to study the epitranscriptome effectively. Further work optimizing the CIRTS using directed evolution will likely yield variants that have improved performance in mammalian systems (Hu et al., 2018). Understanding target site design and effector protein contextual requirements will also improve CIRTS system performance, and a better understanding of the epitranscriptomic pathways being exploited will allow the inventors to design better systems. Additionally, there are a range of other regulatory proteins contained within the human proteome that likely house unique RNA control properties (Dominguez et al., 2018), which can be coupled with CIRTS to create programmable versions of each protein for both functional characterization and potential translational applications. The CIRTS system provides a new approach for studying and exploiting RNA regulation and will open up future opportunities to intervene in cell regulation for disease treatment.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Abudayyeh, O. O., Gootenberg, J. S., Essletzbichler, P., Han, S., Joung, J., Belanto, J. J., Verdine, V., Cox, D. B. T., Kellner, M. J., Regev, A., et al. (2017). RNA targeting with CRISPR-Cas13. Nature 550, 280.
Abudayyeh, O. O., Gootenberg, J. S., Konermann, S., Joung, J., Slaymaker, I. M., Cox, D. B. T., Shmakov, S., Makarova, K. S., Semenova, E., Minakhin, L., et al. (2016). C2c2 is a single-component programmable RNA-guided RNA-targeting CRISPR effector. Science 353, aaf5573.
Adamala, K. P., Martin-Alarcon, D. A., and Boyden, E. S. (2016). Programmable RNA-binding protein composed of repeats of a single modular unit. Proc Natl Acad Sci U S A 113, E2579-2588.
Batra, R., Nelles, D. A., Pirie, E., Blue, S. M., Marina, R. J., Wang, H., Chaim, I. A., Thomas, J. D., Zhang, N., Nguyen, V., et al. (2017). Elimination of Toxic Microsatellite Repeat Expansion RNA by RNA-Targeting Cas9. Cell 170, 899-912 e810.
Blakeley, B. D., and McNaughton, B. R. (2014). Synthetic RNA recognition motifs that selectively recognize HIV-1 trans-activation response element hairpin RNA. ACS Chem Biol 9, 1320-1329.
Chandrasegaran, S., and Carroll, D. (2016). Origins of Programmable Nucleases for Genome Engineering. J Mol Biol 428, 963-989.
Chavez, A., Scheiman, J., Vora, S., Pruitt, B. W., Tuttle, M., P R Iyer, E., Lin, S., Kiani, S., Guzman, C. D., Wiegand, D. J., et al. (2015). Highly efficient Cas9-mediated transcriptional programming. Nat Methods 12, 326.
Choudhury, R., Tsai, Y. S., Dominguez, D., Wang, Y., and Wang, Z. (2012). Engineering RNA endonucleases with customized sequence specificities. Nat Commun 3, 1147.
Chu, V. T., Weber, T., Wefers, B., Wurst, W., Sander, S., Rajewsky, K., and Kuhn, R. (2015). Increasing the efficiency of homology-directed repair for CRISPR-Cas9-induced precise gene editing in mammalian cells. Nat Biotechnol 33, 543-548.
Coller, J., and Wickens, M. (2007). Tethered function assays: an adaptable approach to study RNA regulatory proteins. Methods Enzymol 429, 299-321.
Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339, 819-823.
Cox, D. B. T., Gootenberg, J. S., Abudayyeh, O. O., Franklin, B., Kellner, M. J., Joung, J., and Zhang, F. (2017). RNA editing with CRISPR-Cas13. Science 358, 1019-1027.

Crawford, D. W., Blakeley, B. D., Chen, P. H., Sherpa, C., Le Grice, S. F., Laird-Offringa, I. A., and McNaughton, B. R. (2016). An Evolved RNA Recognition Motif That Suppresses HIV-1 Tat/TAR-Dependent Transcription. ACS Chem Biol 11, 2206-2215.
Cronican, J. J., Beier, K. T., Davis, T. N., Tseng, J. C., Li, W., Thompson, D. B., Shih, A. F., May, E. M., Cepko, C. L., Kung, A. L., et al. (2011). A class of human proteins that deliver functional proteins into mammalian cells in vitro and in vivo. Chem Biol 18, 833-838.
Desjarlais, J. R., and Berg, J. M. (1993). Use of a zinc-finger consensus sequence framework and specificity rules to design specific DNA binding proteins. Proceedings of the National Academy of Sciences 90, 2256.
Dhople, V., Krukemeyer, A., and Ramamoorthy, A. (2006). The human beta-defensin-3, an antibacterial peptide with multiple biological functions. Biochim Biophys Acta 1758, 1499-1512.
Dominguez, D., Freese, P., Alexis, M. S., Su, A., Hochman, M., Palden, T., Bazile, C., Lambert, N. J., Van Nostrand, E. L., Pratt, G. A., et al. (2018). Sequence, Structure, and Context Preferences of Human RNA Binding Proteins. Mol Cell 70, 854-867.e859.
Du, D., Roguev, A., Gordon, D. E., Chen, M., Chen, S.-H., Shales, M., Shen, J. P., Ideker, T., Mali, P., Qi, L. S., et al. (2017). Genetic interaction mapping in mammalian cells using CRISPR interference. Nat Methods 14, 577.
Eberle, A. B., Lykke-Andersen, S., Muhlemann, O., and Jensen, T. H. (2009). SMG6 promotes endonucleolytic cleavage of nonsense mRNA in human cells. Nat Struct Mol Biol 16, 49-55.
Filipovska, A., Razif, M. F., Nygard, K. K., and Rackham, O. (2011). A universal code for RNA recognition by PUF proteins. Nat Chem Biol 7, 425-427.
Fuxman Bass, J. I., Sahni, N., Shrestha, S., Garcia-Gonzalez, A., Mori, A., Bhat, N., Yi, S., Hill, D. E., Vidal, M., and Walhout, A. J. M. (2015). Human gene-centered transcription factor networks for enhancers and disease variants. Cell 161, 661-673.
Gaudelli, N. M., Komor, A. C., Rees, H. A., Packer, M. S., Badran, A. H., Bryson, D. I., and Liu, D. R. (2017). Programmable base editing of A·T to G·C in genomic DNA without DNA cleavage. Nature 551, 464.
Gilbert, L. A., Larson, M. H., Morsut, L., Liu, Z., Brar, G. A., Torres, S. E., Stern-Ginossar, N., Brandman, O., Whitehead, E. H., Doudna, J. A., et al. (2013). CRISPR-mediated modular RNA-guided regulation of transcription in eukaryotes. Cell 154, 442-451.
Glaesner, W., Vick, A. M., Millican, R., Ellis, B., Tschang, S. H., Tian, Y., Bokvist, K., Brenner, M., Koester, A., Porksen, N., et al. (2010). Engineering and characterization of the long-acting glucagon-like peptide-1 analogue LY2189265, an Fc fusion protein. Diabetes Metab Res Rev 26, 287-296.
Gootenberg, J. S., Abudayyeh, O. O., Kellner, M. J., Joung, J., Collins, J. J., and Zhang, F. (2018). Multiplexed and portable nucleic acid detection platform with Cas13, Cas12a, and Csm6. Science 360, 439.
Harrington, L. B., Burstein, D., Chen, J. S., Paez-Espino, D., Ma, E., Witte, I. P., Cofsky, J. C., Kyrpides, N. C., Banfield, J. F., and Doudna, J. A. (2018). Programmed DNA destruction by miniature CRISPR-Cas14 enzymes. Science 362, 839.
Hilton, I.B., D'Ippolito, A. M., Vockley, C. M., Thakore, P. I., Crawford, G. E., Reddy, T. E., and Gersbach, C. A. (2015). Epigenome editing by a CRISPR-Cas9-based acetyltransferase activates genes from promoters and enhancers. Nat Biotechnol 33, 510.
Hockemeyer, D., Wang, H., Kiani, S., Lai, C. S., Gao, Q., Cassady, J. P., Cost, G. J., Zhang, L., Santiago, Y., Miller, J. C., et al. (2011). Genetic engineering of human pluripotent cells using TALE nucleases. Nat Biotechnol 29, 731-734.
Hu, J. H., Miller, S. M., Geurts, M. H., Tang, W., Chen, L., Sun, N., Zeina, C. M., Gao, X., Rees, H. A., Lin, Z., et al. (2018). Evolved Cas9 variants with broad PAM compatibility and high DNA specificity. Nature 556, 57.
Jiang, W., Bikard, D., Cox, D., Zhang, F., and Marraffini, L. A. (2013). RNA-guided editing of bacterial genomes using CRISPR-Cas systems. Nat Biotechnol 31, 233.
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-821.
Joung, J. K., and Sander, J. D. (2012). TALENs: a widely applicable technology for targeted genome editing. Nature Reviews Molecular Cell Biology 14, 49.
Kearns, N. A., Pham, H., Tabak, B., Genga, R. M., Silverstein, N. J., Garber, M., and Maehr, R. (2015). Functional annotation of native enhancers with a Cas9-histone demethylase fusion. Nat Methods 12, 401.
Kim, S., Koo, T., Jee, H. G., Cho, H. Y., Lee, G., Lim, D. G., Shin, H. S., and Kim, J. S. (2018). CRISPR RNAs trigger innate immune responses in human cells. Genome Res.
Kluver, E., Schulz-Maronde, S., Scheid, S., Meyer, B., Forssmann, W. G., and Adermann, K. (2005). Structure-activity relation of human beta-defensin 3: influence of disulfide bonds and cysteine substitution on antimicrobial activity and cytotoxicity. Biochemistry 44, 9804-9816.
Knott, G. J., and Doudna, J. A. (2018). CRISPR-Cas guides the future of genetic engineering. Science 361, 866-869.
Koblan, L. W., Doman, J. L., Wilson, C., Levy, J. M., Tay, T., Newby, G. A., Maianti, J. P., Raguram, A., and Liu, D. R. (2018). Improving cytidine and adenine base editors by expression optimization and ancestral reconstruction. Nat Biotechnol 36, 843-846.
Komor, A. C., Kim, Y. B., Packer, M. S., Zuris, J. A., and Liu, D. R. (2016). Programmable editing of a target base in genomic DNA without double-stranded DNA cleavage. Nature 533, 420.
Konermann, S., Lotfy, P., Brideau, N. J., Oki, J., Shokhirev, M. N., and Hsu, P. D. (2018). Transcriptome Engineering with RNA-Targeting Type VI-D CRISPR Effectors. Cell 173, 665-676.e614.
Liao, H.-K., Hatanaka, F., Araoka, T., Reddy, P., Wu, M.-Z., Sui, Y., Yamauchi, T., Sakurai, M., O'Keefe, D. D., Núñez-Delicado, E., et al. (2017). In Vivo Target Gene Activation via CRISPR/Cas9-Mediated Trans-epigenetic Modulation. Cell 171, 1495-1507.e1415.
Lin, S., Staahl, B. T., Alla, R. K., and Doudna, J. A. (2014). Enhanced homology-directed human genome engineering by controlled timing of CRISPR/Cas9 delivery. Elife 3, e04766.
Liu, L., Li, X., Ma, J., Li, Z., You, L., Wang, J., Wang, M., Zhang, X., and Wang, Y. (2017). The Molecular Architecture for RNA-Guided RNA Cleavage by Cas13a. Cell 170, 714-726 e710.
Maeder, M. L., Linder, S. J., Cascio, V. M., Fu, Y., Ho, Q. H., and Joung, J. K. (2013). CRISPR RNA-guided activation of endogenous human genes. Nat Methods 10, 977-979.
Mali, P., Aach, J., Stranges, P. B., Esvelt, K. M., Moosburner, M., Kosuri, S., Yang, L., and Church, G. M. (2013a). CAS9 transcriptional activators for target specificity screening and paired nickases for cooperative genome engineering. Nat Biotechnol 31, 833-838.
Mali, P., Yang, L., Esvelt, K. M., Aach, J., Guell, M., DiCarlo, J. E., Norville, J. E., and Church, G. M. (2013b). RNA-guided human genome engineering via Cas9. Science 339, 823-826.
Monteys, A. M., Ebanks, S. A., Keiser, M. S., and Davidson, B. L. (2017). CRISPR/Cas9 Editing of the Mutant Huntingtin Allele In Vitro and In Vivo. Mol Ther 25, 12-23.
Montiel-Gonzalez, M. F., Vallecillo-Viejo, I. C., and Rosenthal, J. J. (2016). An efficient system for selectively altering genetic information within mRNAs. Nucleic Acids Res 44, e157.
Nelles, D. A., Fang, M. Y., O'Connell, M. R., Xu, J. L., Markmiller, S. J., Doudna, J. A., and Yeo, G. W. (2016). Programmable RNA Tracking in Live Cells with CRISPR/Cas9. Cell 165, 488-496.
Nishikura, K. (2010). Functions and regulation of RNA editing by ADAR deaminases. Annu Rev Biochem 79, 321-349.
O'Connell, M. R., Oakes, B. L., Sternberg, S. H., East-Seletsky, A., Kaplan, M., and Doudna, J. A. (2014). Programmable RNA recognition and cleavage by CRISPR/Cas9. Nature 516, 263-266.
O'Connell, M. R., Oakes, B. L., Sternberg, S. H., East-Seletsky, A., Kaplan, M., and Doudna, J. A. (2014). Programmable RNA recognition and cleavage by CRISPR/Cas9. Nature 516, 263.
Perez-Pinera, P., Kocak, D. D., Vockley, C. M., Adler, A. F., Kabadi, A. M., Polstein, L. R., Thakore, P. I., Glass, K. A., Ousterout, D. G., Leong, K. W., et al. (2013). RNA-guided gene activation by CRISPR-Cas9-based transcription factors. Nat Methods 10, 973-976.
Qi, L. S., Larson, M. H., Gilbert, L. A., Doudna, J. A., Weissman, J. S., Arkin, A. P., and Lim, W. A. (2013). Repurposing CRISPR as an RNA-guided platform for sequence-specific control of gene expression. Cell 152, 1173-1183.
Rauch, S., He, C., and Dickinson, B. C. (2018). Targeted m(6)A Reader Proteins To Study Epitranscriptomic Regulation of Single RNAs. J Am Chem Soc 140, 11974-11981.
Roundtree, I. A., Evans, M. E., Pan, T., and He, C. (2017). Dynamic RNA Modifications in Gene Expression Regulation. Cell 169, 1187-1200.
Rutkauskas, M., Sinkunas, T., Songailiene, I., Tikhomirova, Maria S., Siksnys, V., and Seidel, R. (2015). Directional R-Loop Formation by the CRISPR-Cas Surveillance Complex Cascade Provides Efficient Off-Target Site Rejection. Cell Reports 10, 1534-1543.
Savić, N., and Schwank, G. (2016). Advances in therapeutic CRISPR/Cas9 genome editing. Translational Research 168, 15-21.
Schierling, B., Dannemann, N., Gabsalilow, L., Wende, W., Cathomen, T., and Pingoud, A. (2012). A novel zinc-finger nuclease platform with a sequence-specific cleavage module. Nucleic Acids Res 40, 2623-2638.
Sinnamon, J. R., Kim, S. Y., Corson, G. M., Song, Z., Nakai, H., Adelman, J. P., and Mandel, G. (2017). Site-directed RNA repair of endogenous Mecp2 RNA in neurons. Proc Natl Acad Sci U S A 114, E9395-E9402.
Sternberg, S. H., Redding, S., Jinek, M., Greene, E. C., and Doudna, J. A. (2014). DNA interrogation by the CRISPR RNA-guided endonuclease Cas9. Nature 507, 62.
Strutt, S. C., Torrez, R. M., Kaya, E., Negrete, O. A., and Doudna, J. A. (2018). RNA-dependent RNA targeting by CRISPR-Cas9. eLife 7, e32724.
Sundaram, G. M., Ismail, H. M., Bashir, M., Muhuri, M., Vaz, C., Nama, S., Ow, G. S., Vladimirovna, I. A., Ramalingam, R., Burke, B., et al. (2017). EGF hijacks miR-198/FSTL1 wound-healing switch and steers a two-pronged pathway toward metastasis. J Exp Med 214, 2889-2900.
Szczelkun, M. D., Tikhomirova, M. S., Sinkunas, T., Gasiunas, G., Karvelis, T., Pschera, P., Siksnys, V., and Seidel, R. (2014). Direct observation of R-loop formation by single RNA-guided Cas9 and Cascade effector complexes. Proceedings of the National Academy of Sciences 111, 9798.
Tambe, A., East-Seletsky, A., Knott, G. J., Doudna, J. A., and O'Connell, M. R. (2018). RNA Binding and HEPN-Nuclease Activation Are Decoupled in CRISPR-Cas13a. Cell Rep 24, 1025-1036.
Vogel, P., Moschref, M., Li, Q., Merkle, T., Selvasaravanan, K. D., Li, J. B., and Stafforst, T. (2018). Efficient and precise editing of endogenous transcripts with SNAP-tagged ADARs. Nat Methods 15, 535-538.
Wagner, D. L., Amini, L., Wendering, D. J., Burkhardt, L.-M., Akyüz, L., Reinke, P., Volk, H.-D., and Schmueck-Henneresse, M. (2018). High prevalence of Streptococcus pyogenes Cas9-reactive T cells within the adult human population. Nat Med.
Wiedenheft, B., Sternberg, S. H., and Doudna, J. A. (2012). RNA-guided genetic silencing systems in bacteria and archaea. Nature 482, 331.
Yan, W. X., Chong, S., Zhang, H., Makarova, K. S., Koonin, E. V., Cheng, D. R., and Scott, D. A. (2018). Cas13d Is a Compact RNA-Targeting Type VI CRISPR Effector Positively Modulated by a WYL-Domain-Containing Accessory Protein. Mol Cell 70, 327-339 e325.
Yi, L., and Li, J. (2016). CRISPR-Cas9 therapeutics in cancer: promising strategies and present challenges. Biochim Biophys Acta 1866, 197-207.
Zhao, B. S., Roundtree, I. A., and He, C. (2017). Post-transcriptional gene regulation by mRNA modifications. Nat Rev Mol Cell Biol 18, 31-42.
Zhou, Z., Dell'Orco, M., Saldarelli, P., Turturo, C., Minafra, A., and Martelli, G.P. (2006). Identification of an RNA-silencing suppressor in the genome of Grapevine virus A. J Gen Virol 87, 2387-2395.
Guilinger, J. P., Thompson, D. B., and Liu, D. R. (2014). Fusion of catalytically inactive Cas9 to FokI nuclease improves the specificity of genome modification. Nat Biotechnol 32, 577-582.
Abil, Z., Denard, C. A., and Zhao, H. (2014). Modular assembly of designer PUF proteins for specific post-transcriptional regulation of endogenous RNA. J Biol Eng 8, 7.
Andreatta, M., and Nielsen, M. (2016). Gapped sequence alignment using artificial neural networks: application to the MHC class I system. Bioinformatics 32, 511-517.
Bao, Z., Jain, S., Jaroenpuntaruk, V., and Zhao, H. (2017). Orthogonal Genetic Regulation in Human Cells Using Chemically Induced CRISPR/Cas9 Activators. ACS Synth Biol 6, 686-693.
Boyle, E. A., Andreasson, J. O. L., Chircus, L. M., Sternberg, S. H., Wu, M. J., Guegler, C. K., Doudna, J. A., and Greenleaf, W. J. (2017). High-throughput biochemical profiling reveals sequence determinants of dCas9 off-target binding and unbinding. Proc Natl

Acad Sci U S A 114, 5461-5466.
Bray, N. L., Pimentel, H., Melsted, P., and Pachter, L. (2016). Near-optimal probabilistic RNA-seq quantification. Nat Biotechnol 34, 525.
Calis, J. J., Maybeno, M., Greenbaum, J. A., Weiskopf, D., De Silva, A. D., Sette, A., Kesmir, C., and Peters, B. (2013). Properties of MHC class I presented peptides that enhance immunogenicity. PLoS Comput Biol 9, e1003266.
Charlesworth, C., Deshpande, P., Dever, D., Dejene, B., Gomez-Ospina, N., Mantri, S., Pavel-Dinu, M., Camarena, J., Weinberg, K., and Porteus, M. Identification of Pre-Existing Adaptive Immunity to Cas9 Proteins in Humans. bioRxiv.
Du, H., Zhao, Y., He, J., Zhang, Y., Xi, H., Liu, M., Ma, J., and Wu, L. (2016). YTHDF2 destabilizes m6A-containing RNA through direct recruitment of the CCR4-NOT deadenylase complex. Nat Commun 7, 12626.
Gao, G., Vandenberghe, L. H., and Wilson, J. M. (2005). New recombinant serotypes of AAV vectors. Curr Gene Ther 5, 285-297.
Gao, Y., Xiong, X., Wong, S., Charles, E. J., Lim, W. A., and Qi, L. S. (2016). Complex transcriptional modulation with orthogonal and inducible dCas9 regulators. Nat Methods 13, 1043-1049.
Jurtz, V., Paul, S., Andreatta, M., Marcatili, P., Peters, B., and Nielsen, M. (2017). NetMHCpan-4.0: Improved Peptide-MHC Class I Interaction Predictions Integrating Eluted Ligand and Peptide Binding Affinity Data. J Immunol 199, 3360-3368.
Kearns, N. A., Pham, H., Tabak, B., Genga, R. M., Silverstein, N. J., Garber, M., and Maehr, R. (2015). Functional annotation of native enhancers with a Cas9-histone demethylase fusion. Nat Methods 12, 401.
Kuttan, A., and Bass, B. L. (2012). Mechanistic insights into editing-site specificity of ADARs. Proc Natl Acad Sci U S A 109, E3295-3304.
Meyer, Kate D., Patil, Deepak P., Zhou, J., Zinoviev, A., Skabkin, Maxim A., Elemento, O., Pestova, Tatyana V., Qian, S.-B., and Jaffrey, Samie R. (2015). 5′ UTR m6A Promotes Cap-Independent Translation. Cell 163, 999-1010.
Moutaftsi, M., Peters, B., Pasquetto, V., Tscharke, D. C., Sidney, J., Bui, H. H., Grey, H., and Sette, A. (2006). A consensus epitope prediction approach identifies the breadth of murine T(CD8+)-cell responses to vaccinia virus. Nat Biotechnol 24, 817-819.
Naso, M. F., Tomkowicz, B., Perry, W. L., 3rd, and Strohl, W. R. (2017). Adeno-Associated Virus (AAV) as a Vector for Gene Therapy. BioDrugs 31, 317-334.
Peters, B., and Sette, A. (2005). Generating quantitative models describing the sequence specificity of biological processes with the stabilized matrix method. BMC Bioinformatics 6, 132.
Pimentel, H., Bray, N. L., Puente, S., Melsted, P., and Pachter, L. (2017). Differential analysis of RNA-seq incorporating quantification uncertainty. Nat Methods 14, 687.
Sidney, J., Assarsson, E., Moore, C., Ngo, S., Pinilla, C., Sette, A., and Peters, B. (2008). Quantitative peptide binding motifs for 19 human and mouse MHC class I molecules derived using positional scanning combinatorial peptide libraries. Immunome Res 4, 2.
Simhadri, V. L., McGill, J., McMahon, S., Wang, J., Jiang, H., and Sauna, Z. E. (2018). Prevalence of Pre-existing Antibodies to CRISPR-Associated Nuclease Cas9 in the USA Population. Mol Ther Methods Clin Dev 10, 105-112.
Vasileva, A., and Jessberger, R. (2005). Precise hit: adeno-associated virus in gene targeting. Nat Rev Microbiol 3, 837-847.
Wang, X., Lu, Z., Gomez, A., Hon, G. C., Yue, Y., Han, D., Fu, Y., Parisien, M., Dai, Q., Jia, G., et al. (2013). N6-methyladenosine-dependent regulation of messenger RNA stability. Nature 505, 117.
Wang, X., Zhao, Boxuan S., Roundtree, Ian A., Lu, Z., Han, D., Ma, H., Weng, X., Chen, K., Shi, H., and He, C. (2015). N6-methyladenosine Modulates Messenger RNA Translation Efficiency. Cell 161, 1388-1399.
Wu, Z., Yang, H., and Colosi, P. (2010). Effect of genome size on AAV vector packaging. Mol Ther 18, 80-86.
Xiao, W., Adhikari, S., Dahal, U., Chen, Y.-S., Hao, Y.-J., Sun, B.-F., Sun, H.-Y., Li, A., Ping, X.-L., Lai, W.-Y., et al. (2016). Nuclear m6A Reader YTHDC1 Regulates mRNA Splicing. Mol Cell 61, 507-519.
Yan, W. X., Hunnewell, P., Alfonse, L. E., Carte, J. M., Keston-Smith, E., Sothiselvam, S., Garrity, A. J., Chong, S., Makarova, K. S., Koonin, E. V., et al. (2019). Functionally diverse type V CRISPR-Cas systems. Science 363, 88-91.

1. A RNA regulatory system comprising at least one of each:
i) a RNA hairpin binding domain;
ii) a RNA targeting molecule comprising a RNA targeting region and at least one hairpin structure, wherein the hairpin structure of the RNA targeting molecule specifically binds to i; and
iii) a RNA regulatory domain.

2. The system of claim 1, wherein the RNA hairpin binding domain and the RNA regulatory domain are operably linked.

3. The system of claim 1, wherein parts i), ii), and/or iii) are human or are human-derived.

4. The system of claim 2 or 3, wherein i) and iii) are operably linked through a peptide bond.

5. The system of claim 2 or 3, wherein i) and iii) are operably linked through non-covalent interactions.

6. The system of any one of claims 1-5, wherein the RNA regulatory domain is covalently linked to a first dimerization domain and the RNA hairpin binding domain is covalently linked to a second dimerization domain and wherein the first and second dimerization domain are capable of dimerizing to form a non-covalent or covalent linkage.

7. The system of claim 6, wherein the dimerization is inducible.

8. The system of claim 7, wherein the dimerization comprises ligand-induced dimerization.

9. The system of claim 8, wherein one of the first or second dimerization domain comprises PYR/PYR1-like (PYL1), the other of the first or second domain comprises ABA insensitive 1 (ABI1), and the ligand comprises abscisis acid (ABA) or derivatives or fragments thereof.

10. The system of claim 8, wherein the first and/or second dimerization domain comprises FKBP12 and the ligand comprises FK1012 or derivatives or fragments thereof.

11. The system of claim 8, wherein one of the first or second dimerization domains comprises FK506 binding protein (FKBP), the other of the first or second domain comprises FKBP-Rap binding domain of mammalian target of Rap mTOR (Frb), and the ligand comprises rapamycin (Rap) or derivatives or fragments thereof.

12. The system of any one of claims 1-11, wherein ii) comprises at least two hairpin structures.

13. The system of any one of claims 1-12, wherein ii) comprises one or more modified nucleotides.

14. The system of any one of claims 1-13, wherein the system further comprises a stabilizer polypeptide; wherein the stabilizer polypeptide comprises a cationic polypeptide that binds non-specifically to nucleic acids.

15. The system of claim 14, wherein the stabilizer polypeptide is human-derived.

16. The system of any one of claims 1-15, wherein the total size of the system is less than 150 kDa.

17. The system of any one of claims 1-16, wherein i) comprises U1A, SLBP, or variants thereof.

18. The system of any one of claims 1-17, wherein ii) comprises a TAR hairpin scaffold of SEQ ID NO:1.

19. The system of any one of claims 1-18, wherein ii) comprises a SLBP hairpin scaffold of SEQ ID NO:2.

20. The system of any one of claims 1-19, wherein ii) comprises a linker.

21. The system of claim 20, wherein the linker is at least 5 amino acids.

22. The system of any one of claims 1-21, wherein the RNA targeting region comprises at least 12 nucleotides.

23. The system of any one of claims 1-22, wherein iii) comprises a nuclease, methylase, demethylase, translational activator, translational repressor, single-stranded RNA cleavage activity, double-stranded RNA cleavage activity, or RNA binding activity.

24. The system of any one of claims 1-23, wherein iii) comprises a Pin nuclease domain or a m6A reader protein or portion thereof.

25. The system of claim 24, wherein iii) comprises YTHDF1, YTHDF2, or ADAR.

26. The system of any one of claims 14-25, wherein the stabilizer protein comprises HBEGF, beta-defensin, or variants or portions thereof.

27. The system of any one of claims 1-26, wherein the RNA targeting region of ii) hybridizes to a target RNA in a prokaryotic or eukaryotic cell.

28. The system of any one of claims 1-27, wherein i) and/or iii) comprises one or more nuclear localization signals (NLS)s.

29. The system of any one of claims 1-28, wherein the system comprises at least two of each i, ii, and iii.

30. The system of any one of claims 1-29, wherein the RNA regulatory domain cleaves RNA, promotes RNA translation, inhibits RNA translation, or modifies the base sequence of RNA.

31. A vector system comprising one or more nucleic acid vectors comprising a nucleotide encoding: i) a RNA hairpin binding domain; ii) a RNA targeting molecule comprising a RNA targeting region and at least one hairpin structure, wherein the hairpin structure of the RNA targeting molecule specifically binds to i), and iii) a RNA regulatory domain.

32. The vector system of claim 31, wherein the RNA hairpin binding domain and the RNA regulatory domain are operably linked.

33. The vector system of claim 31 further comprising a regulatory element operably linked to the nucleotide encoding i, ii, and/or iii.

34. The vector system of claim 31 or 33, wherein the one or more nucleic acid vectors are optimized for expression in an eukaryotic cell.

35. The vector system of any one of claims 31-34, wherein the expression is constitutive or conditional.

36. The vector system of any one of claims 31-35, wherein i, ii, and iii are on a single vector.

37. The vector system of any one of claims 31-36, wherein one or more of the vectors are viral vectors.

38. The vector system of claim 31-37, wherein the one or more vectors comprise one or more retroviral, lentiviral, adenoviral, adeno-associated or herpes simplex viral vectors.

39. The vector system of any one of claims 31-36, wherein one or more of the vectors are non-viral vectors.

40. A conjugate comprising a RNA regulatory domain operably linked to a RNA targeting molecule, wherein the RNA targeting molecule comprises a RNA targeting region and at least one hairpin structure.

41. The conjugate of claim 40, wherein the RNA regulatory domain is human derived.

42. The conjugate of claim 40 or 41, wherein the RNA regulatory domain and the RNA targeting molecule are operably linked through a peptide bond.

43. The conjugate of any one of claims 40-42, wherein the polypeptide further comprises one or more linkers.

44. The conjugate of claim 40 or 41, wherein the RNA regulatory domain and the RNA targeting molecule are operably linked through non-covalent interactions.

45. The conjugate of any one of claims 40-44, wherein the RNA regulatory domain is covalently linked to a first dimerization domain and the RNA targeting molecule is covalently linked to a second dimerization domain and wherein the first and second dimerization domain are capable of dimerizing to form a non-covalent or covalent linkage.

46. The conjugate of claim 45, wherein the dimerization is inducible.

47. The conjugate of claim 46, wherein the dimerization comprises ligand-induced dimerization.

48. The conjugate of claim 47, wherein one of the first or second dimerization domains comprises PYR/PYR1-like (PYL1), the other of the first or second domain comprises ABA insensitive 1 (ABI1), and the ligand comprises abscisis acid (ABA) or derivatives or fragments thereof.

49. The conjugate of claim 47, wherein the first and/or second dimerization domain comprises FKBP12 and the ligand comprises FK1012 or derivatives or fragments thereof.

50. The conjugate of claim 47, wherein one of the first or second dimerization domains comprises FK506 binding protein (FKBP), the other of the first or second domain comprises FKBP-Rap binding domain of mammalian target of Rap mTOR (Frb), and the ligand comprises rapamycin (Rap) or derivatives or fragments thereof.

51. The conjugate of any one of claims 40-50, wherein the RNA targeting molecule comprises at least two hairpin structures.

52. The conjugate of any one of claims 40-51, wherein the RNA targeting molecule comprises one or more modified nucleotides.

53. The conjugate of any one of claims 40-52, wherein the RNA targeting molecule comprises a TAR hairpin scaffold of SEQ ID NO:1.

54. The conjugate of any one of claims 40-53, wherein the RNA targeting molecule comprises a SLBP hairpin scaffold of SEQ ID NO:2.

55. The conjugate of any one of claims 40-54, wherein the RNA targeting molecule comprises a linker.

56. The conjugate of claim 55, wherein the linker comprises at least 5 amino acids.

57. The conjugate of any one of claims 40-56, wherein the RNA targeting region comprises at least 12 nucleotides.

58. The conjugate of any one of claims 40-57, wherein the RNA regulatory domain comprises a nuclease, methylase, demethylase, translational activator, translational repressor, single-stranded RNA cleavage activity, double-stranded RNA cleavage activity, or RNA binding activity.

59. The conjugate of any one of claims 40-57, wherein the RNA regulatory domain comprises a Pin nuclease domain or a m6A reader protein or portion thereof.

60. The conjugate of any one of claims 40-59, wherein the RNA regulatory domain comprises YTHDF1, YTHDF2, or ADAR.

61. The conjugate of any one of claims 40-60, wherein the RNA targeting region of the RNA targeting molecule hybridizes to a target RNA in a prokaryotic or eukaryotic cell.

62. The conjugate of any one of claims 40-61, wherein the conjugate comprise one or more nuclear localization signals (NLS)s.

63. The conjugate of any one of claims 40-62, wherein the RNA regulatory domain cleaves RNA, promotes RNA translation, inhibits RNA translation, or modifies the base sequence of RNA.

64. A fusion protein comprising a RNA hairpin binding domain and a RNA regulatory domain.

65. A fusion protein comprising a RNA regulatory domain and a first dimerization domain.

66. A fusion protein comprising a RNA hairpin binding domain and a second dimerization domain.

67. The fusion protein of claim 65 or 66, wherein dimerization of the first and/or second dimerization domain is inducible.

68. The fusion protein of claim 67, wherein the dimerization comprises ligand-induced dimerization.

69. The fusion protein of claim 68, wherein the first and second dimerization domains are selected from PYL1 and ABI1 and the ligand comprises ABA or derivatives or fragments thereof.

70. The fusion protein of claim 68, wherein the first and/or second dimerization domain comprises FKBP12 and the ligand comprises FK1012 or derivatives or fragments thereof.

71. The fusion protein of claim 68, first and second dimerization domains are selected from FKBP and Frb, and the ligand comprises rapamycin or derivatives or fragments thereof.

72. The fusion protein of any one of claims 64-71, wherein the RNA hairpin binding domain and/or RNA regulatory domain are human-derived.

73. The fusion protein of any one of claims claim 64-72, wherein the fusion protein is less than 150 kDa.

74. The fusion protein of any one of claims 64-73, wherein the RNA hairpin binding domain comprises U1A, SLBP, or variants or fragments thereof.

75. The fusion protein of any one of claims 64-74, wherein the RNA regulatory domain comprises a nuclease, methylase, demethylase, translational activator, translational repressor, single-stranded RNA cleavage activity, double-stranded RNA cleavage activity, or RNA binding activity.

76. The fusion protein of claim 75, wherein the RNA regulatory domain comprises a Pin nuclease domain or a m6A reader protein or portion thereof.

77. The fusion protein of claim 76, wherein the RNA regulatory domain comprises YTHDF1 or YTHDF2.

78. The fusion protein of any one of claims 64-77 further comprising one or more nuclear localization signals (NLS)s.

79. The fusion protein of any one of claims 64-77, wherein the RNA regulatory domain cleaves RNA, promotes RNA translation, inhibits RNA translation, or modifies the base sequence of RNA.

80. A nucleic acid encoding the fusion protein of any one of claims 64-79.

81. A delivery vehicle comprising the system of any one of claims 1-39, the conjugate of any one of claims 40-63, or the fusion protein of any one of claims 64-79.

82. The delivery vehicle of claim 81, wherein the delivery vehicle comprises liposome(s), particle(s), exosome(s), microvesicle(s), a gene-gun or one or more nucleic acid vector(s).

83. A composition comprising the system of any one of claims 1-39, the conjugate of any one of claims 40-63, the fusion protein of any one of claims 64-79, or the delivery vehicle of any one of claims 81-82.

84. A cell comprising the system of any one of claims 1-39, the conjugate of any one of claims 40-63, the fusion protein of any one of claims 64-79, the delivery vehicle of any one of claims 81-82, or the composition of claim 83.

85. A method of modulating at least one target RNA comprising contacting the target RNA with the system of any one of claims 1-39, the conjugate of any one of claims 40-63, the fusion protein of any one of claims 64-79, the delivery vehicle of any one of claims 81-82, or the composition of claim 83.

86. The method of claim 85, wherein modulating the at least one target RNA comprises cleaving, demethylating, methylating, activating translation, repressing translation, promoting degradation, and/or binding to the RNA.

87. The method of claim 85 or 86, wherein the target RNA is in a prokaryotic or eukaryotic cell.

88. The method of claim 87, wherein the target RNA is in a human cell.

89. The method of claim 87 or 88, wherein the target RNA is in vitro or in vivo.

90. A cell or progeny thereof comprising modulated target RNA, wherein the target RNA has been modulated according to any one of claims 85-89.

91. A multicellular organism comprising one or more cells according to claim 90.

92. A plant or animal comprising one or more cells according to claim 91.

93. A kit comprising the system of any one of claims 1-39, the conjugate of any one of claims 40-63, the fusion protein of any one of claims 64-79, the delivery vehicle of any one of claims 81-82, or the composition of claim 83.

94. A method for modulating a target RNA in a subject, the method comprising administering the conjugate of any one of claims 40-63, the fusion protein of any one of claims 64-79, the delivery vehicle of any one of claims 81-82, or the composition of claim 83.
